(19) United States
(12) Patent Application Publication
(10) Pub. No.: US 2010/0143340 A1
(43) Pub. Date: Jun. 10, 2010

(54) METHODS AND COMPOSITIONS FOR TREATING CANCER

(75) Inventors: Parag Kolhe, Chesterfield, MO (US); Vinay Radhakrishnan, Thousand Oaks, CA (US); Leonore Witchey-Lakshmanan, Piscataway, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/518,405

(22) PCT Filed: Dec. 11, 2007
(86) PCT No.: PCT/US07/25321

§ 371 Date: Feb. 24, 2010

Related U.S. Application Data
(60) Provisional application No. 60/874,641, filed on Dec. 13, 2006.
(60) Provisional application No. 60/972,504, filed on Sep. 14, 2007.
(60) Provisional application No. 60/974,241, filed on Sep. 21, 2007.
(60) Provisional application No. 60/979,269, filed on Oct. 11, 2007.

(51) Int. Cl. A61K 39/395

(52) U.S. Cl. 424/131.1; 424/130.1; 424/141.1; 424/133.1

(57) ABSTRACT

The present invention provides methods for preventing or treating a medical disorder in a subject comprising administering to the subject an effective amount of a stable pharmaceutical formulation comprising an antibody or antigen-binding fragment thereof.

This application claims the benefit of U.S. provisional patent application No. 60/874,641; filed Dec. 13, 2006; 60/972,504; filed Sep. 14, 2007; 60/974,241; filed Sep. 21, 2007; and 60/979,269; filed Oct. 11, 2007; each of which is herein incorporated by reference in its entirety.

### FIELD OF THE INVENTION

The present invention provides, inter alia, methods for treating or preventing a medical disorder mediated by IGF-1R, IGF-1 and/or IGF-2 in a subject comprising administering to the subject a therapeutically effective amount of a pharmaceutical formulation comprising an antibody which exhibits high stability.

### BACKGROUND OF THE INVENTION

Antibodies, like most proteins, must maintain their higher order structure in order to maintain their activity. One problem faced by companies selling antibodies, including therapeutic antibodies, is the identification of conditions under which the antibody can exist for an extended period of time without denaturing and, thus, losing biological activity. In general, therapeutic antibodies on the market are relatively unstable, requiring careful handling and storage at low temperatures. For example, the therapeutic antibodies Avastin™, Herceptin® and Erbitux™ require storage at 2° C. to 8° C. It is likely that the anti-IGF1R antibodies owned by various companies in the industry (e.g., Pfizer, Imclone, Pierre Fabre, Roche and Immunogen) will, similarly, exhibit instability.

The low level of stability exhibited by currently available therapeutic antibodies is disadvantageous due both to the cost and inconvenience presented by the special storage conditions required as well as to the danger of accidental inactivation of the antibody before administration and possible toxicity/immunogenicity due to the degradation/aggregation. There is, thus, a need in the art for a pharmaceutical formulation that will allow therapeutic antibodies, for example anti-IGF1R therapeutic antibodies, to be stable while stored at a wide range of conditions. Providing methods for treating or preventing medical conditions which are mediated by IGF-1R, IGF-1 and/or IGF-2 comprising administration of these pharmaceutical formulations would also fulfill a significant need in the art for a satisfactory treatment of cancer, and specifically cancers mediated by IGF1R, IGF-1 and/or IGF-2.

### SUMMARY OF THE INVENTION

The present invention addresses the above-referenced need in the art by providing methods for treating or preventing a medical disorder in a subject comprising administration to the subject a therapeutically effective amount of a pharmaceutical formulation, wherein the pharmaceutical formulation comprises an isolated anti-IGF1R antibody (e.g., monoclonal antibody) or an antigen-binding fragment thereof, that exhibits superior stability.

The present invention provides a method for treating or preventing a medical condition mediated by expression or activity of IGF1R comprising administering a dosage of an antibody or antigen-binding fragment thereof (e.g., a monoclonal antibody, labeled antibody, bivalent antibody, a polyclonal antibody, a bispecific antibody, a chimeric antibody, a recombinant antibody, an anti-idiotypic antibody, a humanized antibody or a bispecific antibody, a camelized single domain antibody, a diabody, an scfv, an scfv dimer, a dsfv, a (dsfv)₂, a dsFv-dsfv′, a bispecific ds diabody, an Fv, an Fab, an Fab′, an F(ab′)₂, or a domain antibody) which binds specifically to IGF1R (optionally in association with a further chemotherapeutic agent such as lonafarnib; cetuximab; irinotecan; erlotinib; rapamycin; temsirolimus; sorafenib; gefitinib; fulvestrant; octreotide; temozolomide; or 4-hydroxytamoxifen) which dosage achieves and maintains a blood concentration of at least about 19 μg/mL, e.g., a dosage of about 10 mg/kg body weight or more; administered once every 3 weeks or more frequently (e.g., once every week or 2 weeks). In an embodiment of the invention, the medical condition is a member selected from the group consisting of osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia (e.g., B-precursor type or T-cell type), acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease. In an embodiment of the invention, the antibody or fragment comprises one or more members selected from the group consisting of: (a) CDR-L1, CDR-L2 and CDR-L3 of the variable region of the 19D12/15H12 light chain immunoglobulin, and (b) CDR-H1, CDR-H2 and CDR-H3 of the variable region of the 19D12/15H12 heavy chain immunoglobulin. For example, in an embodiment of the invention, the antibody or antigen-binding fragment thereof comprises a light chain immunoglobulin comprising complementarity determining regions comprising the amino acid sequences:

| | |
|---|---|
| RASQSIGSSLH; | (SEQ ID NO: 1) |
| YASQSLS; | (SEQ ID NO: 2) |
| and | |
| HQSSRLPHT; | (SEQ ID NO: 3) |

and a heavy chain immunoglobulin comprising complementarity determining regions comprising the amino acid sequences:

| | |
|---|---|
| SFAMH; | (SEQ ID NO: 4) |
| VIDTRGATYYADSVKG; | (SEQ ID NO: 6) |
| and | |
| LGNFYYGMDV. | (SEQ ID NO: 7) |

In an embodiment of the invention, the antibody or antigen-binding fragment thereof comprises: (a) a light chain immunoglobulin comprising a mature fragment of the amino acid sequence set forth in SEQ ID NO: 8, 9, 10, 11, 12, 13 or 14; or (b) a heavy chain immunoglobulin comprising a mature fragment of the amino acid sequence set forth in SEQ ID NO: 15, 16 or 17; or both. Embodiments of the invention include those wherein the antibody or antigen-binding fragment thereof comprises a light chain immunoglobulin comprising amino acids 20-128 of the amino acid sequence set forth in SEQ ID NO: 14 and a heavy chain immunoglobulin comprising amino acids 20-137 of the amino acid sequence set forth in SEQ ID NO: 16. In an embodiment of the invention, the further chemotherapeutic agent is one or more members selected from the group consisting of:

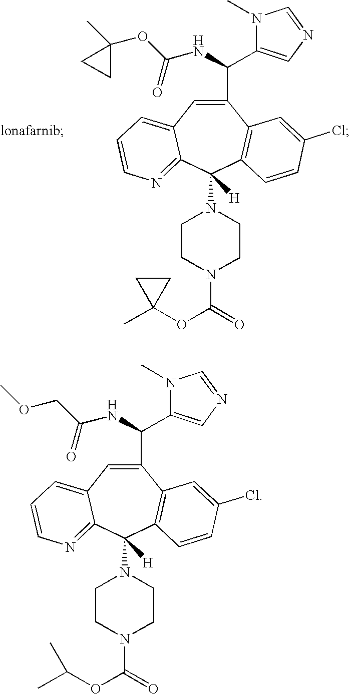

### BMS-214662

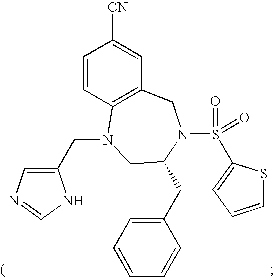

tipifarnib; HuMax-CD20; HuMax-EGFr; bevacizumab; Ibritumomab tiuxetan; a mixture of tositumomab and Iodine I¹³¹; gemtuzumab ozogamicin; MDX-010; CP-724714; TAK-165; HKI-272; gefitinib; erlotinib; calcitriol, lapatanib; GW2016; canertinib; ABX-EGF antibody; cetuximab; EKB-569; PKI-166; GW-572016; PD166285; goserelin acetate; triptorelin pamoate; the FOLFOX regimen; 5′-deoxy-5-fluorouridine; Asparaginase; Bacillus Calmette-Guerin (BCG) vaccine; bleomycin; buserelin; busulfan; oxaliplatin; JM118; JM383; JM559; JM518;

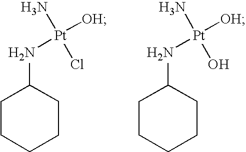

satraplatin; carboplatin; diethylstilbestrol; estradiol; conjugated estrogens; cladribine; clodronate; cyclophosphamide; cyproterone; cytarabine; dacarbazine; dactinomycin; PTK787; ZK 222584; VX-745; PD 184352; rapamycin; or temsirolimus; LY294002; LY292223; LY292696; LY293684; LY293646; sorafenib; ZM336372; L-779,450; flavopiridol; UCN-01;

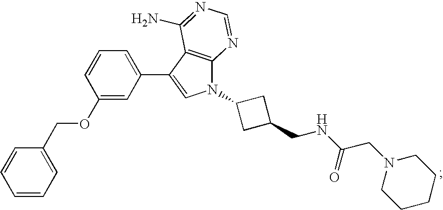

amifostine; NVP-LAQ824; suberoyl analide hydroxamic acid; valproic acid; trichostatin A; FK-228; SU11248; medroxyprogesterone acetate; hydroxyprogesterone caproate; 17-((1-Oxohexyl)oxy)pregn-4-ene-3,20-dione; carmustine; chlorambucil; octreotide; bortezomib; paclitaxel; docetaxel; vincristine; vinblastine; epothilone B; BMS-247550; etoposide; BMS-310705; temozolomide; 8-carbamoyl-3-methyl-[3H]-imidazo[5,1-d]-1,2,3,5-tetrazin-4-one; 8-carbamoyl-3-n-propyl-[3H]-imidazo[5,1-d]-1,2,3,5-tetrazin-4-one; 8-carbamoyl-3-(2-chloroethyl)-[3H]-imidazo-[5,1-d]-1,2,3,5-tetrazin-4-one; 3-(2-chloroethyl)-8-methylcarbamoyl-[3H]-imidazo[5,1-d]-1,2,3,5-tetrazin-4-one; 8-carbamoyl-3-(3-chloropropyl)-[3H]-imidazo-[5,1-d]-1,2,3,5-tetrazin-4-one; 8-carbamoyl-3-(2,3-dichloropropyl)-[3H]-imidazo[5,1-d]-1,2,3,5-tetrazin-4-one; 3-allyl-8-carbamoyl-[3H]-imidazo[5,1-d]-1,2,3,5-tetrazin-4-one; 3-(2-chloroethyl)-8-dimethylcarbamoyl-[3H]-imidazo[5,1-d]-1,2,3,5-tetrazin-4-one; 3-(2-bromoethyl)-8-carbamoyl-[3H]-imidazo-[5,1-d]-1,2,3,5-tetrazin-4-one; 3-benzyl-8-carbamoyl-[3H]-imidazo[5,1-d]-1,2,3,5-tetrazin-4-one; 8-carbamoyl-3-(2-methoxyethyl)-[3H]-imidazo[5,1-d]-1,2,3,5-tetrazin-4-one; 8-carbamoyl-3-cyclohexyl-[3H]-imidazo[5,1-d]-1,2,3,5-tetrazin-4-one; 8-carbamoyl-3-(methoxybenzyl)-[3H]imidazo[5,1-d]-1,2,3,5-tetrazin-4-one; doxorubicin; daunorubicin; epirubicin; bicalutamide; flutamide; nilutamide; megestrol acetate; hydroxyurea; Idarubicin; ifosfamide; imatinib; leucovorin; leuprolide; levamisole; lomustine; mechlorethamine; melphalanm; mercaptopurine; mesna; methotrexate; mitomycin; mitotane; mitoxantrone; fludarabine; fludrocortisone; fluoxymesterone; KRN951; aminoglutethimide; amsacrine; anagrelide; droloxifene, 4-hydroxytamoxifen; tamoxifen; pipendoxifene; arzoxifene; raloxifene; fulvestrant; acolbifene; toremifine; lasofoxifene; idoxifene; bazedoxifene; HMR-3339; ZK-186619; anastrazole; letrozole; exemestane; gemcitabine HCl; 13-cis-retinoic acid; pamidronate; pentostatin; Plicamycin; porfimer; procarbazine; raltitrexed; Rituximab streptozocin; teniposide; testosterone; thalidomide; thioguanine; thiotepa; tretinoin vindesine; interferon alfa-2^{a}; interferon alfa-2b; interferon alfa-2c; interferon alfa n-1; interferon alfa n-3; consensus interferon; albumin-interferon-alpha; camptothecin; topotecan; etoposide; irinotecan; AEW-541;

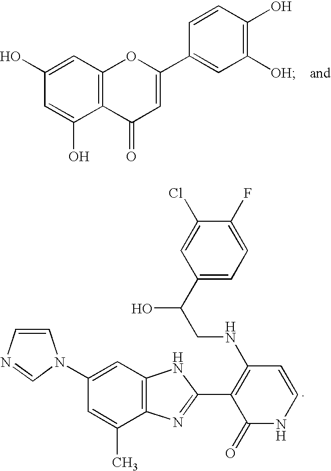

In an embodiment of the invention, the antibody or antigen-binding fragment thereof is linked to a constant region such as a κ light chain, a γ1 heavy chain, a γ2 heavy chain, a γ3 heavy chain or a γ4 heavy chain. In an embodiment of the invention, the antibody or antigen-binding fragment thereof is an isolated antibody comprising a heavy chain encoded by a polynucleotide in plasmid 15H12/19D12 HCA (γ1) which is deposited at the American Type Culture Collection (ATCC) under number PTA-5216; and a light chain encoded by a polynucleotide in plasmid 15H12/19D12 LCF (κ) which is deposited at the American Type Culture Collection (ATCC) under number PTA-5220.

The present invention provides a unit dosage form comprising a one or more doses of a pharmaceutically acceptable carrier and an antibody or antigen-binding fragment thereof comprising one or more members selected from the group consisting of: (a) CDR-L1, CDR-L2 and CDR-L3 of the variable region of the 19D12/15H12 light chain immunoglobulin, and (b) CDR-H1, CDR-H2 and CDR-H3 of the variable region of the 19D12/15H12 heavy chain immunoglobulin; wherein said dose is sufficient to reach and maintain a 19 μg/mL blood concentration of said antibody or fragment when administered once every three weeks or more frequently. For example, in an embodiment of the invention, the dosage form is acceptable for parenteral administration, e.g., intravenous, intramuscular, intratumoral, intrathecal, intraarterial and subcutaneous. In an embodiment of the invention, the unit dosage form is aqueous or lyophilized. The scope of the present invention also includes those wherein the unit dosage form is in a vial, such as a glass vial or a hypodermic needle.

### BRIEF DESCRIPTION OF THE FIGURES

FIG. 1. (a) representative FUV CD scan of anti-IGF1R antibody in acetate buffer of pH 5; (b) representative NUV CD scan of anti-IGF1R antibody in acetate buffer of pH 5.

FIG. 2. (a) Far UV CD Spectrum of anti-IGF1R antibody in various buffers; (b) Change in ellipticity at 217 nm as a function of pH; (c) Change in ellipticity at 235 nm as a function of pH; (d) Change in ellipticity at 235 nm as a function of pH.

FIG. 3. Near UV CD Spectra of anti-IGF1R antibody in various buffers.

FIG. 4. (a) FUV CD Thermal melt data for anti-IGF1R antibody; (b) Tₒₙₛₑₜ (from FUV CD data) as a function of pH.

FIG. 5. (a) NUV CD Thermal melt data for anti-IGF1R antibody; (b) Tₒₙₛₑₜ (from NUV CD data) as a function of pH.

FIG. 6. (a) DSC thermograms for anti-IGF1R antibody; (b) Tₒₙₛₑₜ (from DSC data) as a function of pH; (c) Tₘ₁ (from DSC data) as a function of pH.

FIG. 7. (a) Particle size distribution of anti-IGF1R antibody; (b) Change in size distribution of anti-IGF1R antibody (in phosphate buffer of pH 7) at various temperatures.

FIG. 8. (a) Tₒₙₛₑₜ of aggregation data for anti-IGF1R antibody; (b) Tₒₙₛₑₜ of aggregation as a function of pH.

FIG. 9. (Tₒₙₛₑₜ from FUV CD data): Effect of Sodium Chloride on Tₒₙₛₑₜ.

FIG. 10. (Tₒₙₛₑₜ from FUV CD data): Effect of Sucrose on Tₒₙₛₑₜ.

FIG. 11. Stability of the anti-IGF1R antibody in acetate buffer at pH 5.5 with 7% w/v sucrose.

FIG. 12. Mean anti-IGF1R antibody LCF/HCA Serum Concentrations (Log-Linear and Linear-Linear) following a single IV infusion of 0.3, 1, 3, 10, or 20 mg/kg anti-IGF1R antibody LCF/HCA to healthy volunteers.

### DETAILED DESCRIPTION OF THE INVENTION

The present invention provides methods for treating or preventing medical disorders mediated by IGF-1R, IGF-1 and/or IGF-2 in a subject comprising administering to the subject a therapeutically effective amount of a stable pharmaceutical formulation comprising an isolated antibody or antigen-binding fragment thereof that binds specifically to IGF-1R, a buffer and sucrose. The invention provides stable antibody formulations for use in these methods and pharmacokinetic studies supporting specific dosing regimens. The data from these studies demonstrate that the stable antibody formulations of the invention are effective at inhibiting tumor growth in a xenograph model. Further studies in cynomolgus monkeys show that the formulations have low toxicity at the dosage levels required to maintain the therapeutic concentrations established in the xenograph studies.

Antibodies in the formulations used in the methods of the present invention exhibit superior stability. The formulations provided allow antibodies contained in them to remain intact even after several months of storage at room temperature (e.g., 25° C.). Such high stability makes the formulations of the invention particularly useful, for example, because the formulations allow the clinician, patient or pharmacy possessing the formulation to choose conveniently between storage at room temperature or under refrigeration. Moreover, the high stability ensures that the antibodies retain their biological activity over time which, in turn, ensures that they retain their efficacy e.g., when used to treat a cancerous condition. The particular benefits of the formulations of the invention can be realized even in the absence of storage at room temperature (e.g., under refrigeration at 4° C.). When stored at 4° C., the formulations exhibit somewhat greater stability.

The present invention provides, inter alia, methods for treating and preventing medical disorders comprising administration of a pharmaceutical formulation, wherein the pharmaceutical formulation comprises any anti-IGF1R antibody, a buffer such as acetate/acetic acid buffer and sucrose at about pH 5.5 to about 6.0 (e.g., 5.5., 5.6, 5.7, 5.8, 5.9, 6.0; in an embodiment of the invention, pH is about 5.3 or 5.4). The formulation of the present invention is useful, for example, for administration to a patient for the treatment or prevention of any medical disorder mediated by elevated expression or activity of IGF1R or by elevated expression of its ligand (e.g., IGF-I or IGF-II) and which may be treated or prevented by modulation of IGF1R ligand binding, activity or expression. In an embodiment of the invention, the disease or condition is mediated by an increased level of IGF1R, IGF-I or IGF-II and is treated or prevented by decreasing IGF1R ligand binding, activity (e.g., autophosphorylation activity) or expression.

In an embodiment of the invention, the formulation of the invention is as set forth below:

| Ingredient | mg/mL |
|---|---|
| Anti IGF1R antibody (API) | 20.0 |
| Sodium Acetate Trihydrate USP | 2.30 |
| Glacial Acetic Acid USP/Ph. Eur | 0.18 |
| Sucrose NF, Ph. Eur, BP | 70.0 |
| Water for Injection USP, Ph. Eur. | q.s. ad 1 mL |

For general information concerning formulations, see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York, Kenneth A. Walters (ed.) (2002) Dermatological and Transdermal Formulations (Drugs and the Pharmaceutical Sciences), Vol 119, Marcel Dekker.

The term “subject” or “patient” includes any organism, for example, a mammal (e.g., rat, mouse, cat, dog, horse, rabbit, monkey, ape, primate, chimpanzee, bird or cow) such as a human including pediatric (e.g., 1, 2, 3, 4, 5, 6, 7, 8, 9, 10, 11, 12, 13, 14, 15, 16, 17 or 18 years of age) and geriatric subjects (e.g., 60, 65, 70, 75, 80, 85, 90 or more years of age) thereof.

Medical disorder mediated by IGF1R, IGF-1 and/or IGF-2 (e.g., the activity (e.g., kinase activity and/or ligand or receptor binding activity) or expression thereof) include, for example, any of acromegaly, bladder cancer, Wilm's cancer, ovarian cancer, pancreatic cancer, benign prostatic hyperplasia, breast cancer, prostate cancer, bone cancer, lung cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, any haemotological malignancy (e.g., chronic lymphoblastic leukemia, chromic myelomonocytic leukemia, acute lymphoblastic leukemia (e.g., B-precursor type or T-cell type), acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders), and central nervous system tumors (e.g., brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma), myeloproliferative disorders (e.g., polycythemia vera, thrombocythemia, idiopathic myelfibrosis), soft tissue sarcoma, thyroid cancer, endometrial cancer, carcinoid cancer, germ cell tumors or liver cancer

### Antibodies

The methods of the present invention comprises administration of a pharmaceutical composition comprising an anti-IGF1R antibody or antigen-binding fragment thereof. The term “anti-IGF1R” antibody includes any antibody comprising e.g., 15H12/19D12 HC (heavy chain), HCA or HCB and/or 15H12/19D12 LC (light chain), LCA, LCB, LCC, LCD, LCE or LCF (or any mature fragment thereof) (e.g., LCF and HCA). An anti-IGF1R antibody or antigen-binding fragment thereof includes, in an embodiment of the invention, antibodies and fragments that bind specifically to IGF1R or any fragment thereof (e.g., sIGF1R). Antibodies include, in an embodiment of the invention, monoclonal antibodies, polyclonal antibodies, humanized antibodies, chimeric antibodies, anti-idiotypic antibodies and bispecific antibodies and fragments include Fab antibody fragments, F(ab)₂ antibody fragments, Fv antibody fragments (e.g., V_{H} or V_{L}), single chain Fv antibody fragments and dsFv antibody fragments. Furthermore, the anti-IGF1R antibodies administered in the methods of the invention, in one embodiment, are fully human antibodies. In an embodiment, the anti-IGF1R antibody is a monoclonal, fully human antibody. In an embodiment of the invention, the anti-IGF1R antibody includes one or more of the variable regions and/or CDRs whose amino acid and nucleotide sequences are set forth herein:

| | |
|---|---|
| RASQSIGSSLH; | (SEQ ID NO: 1) |
| YASQSLS; | (SEQ ID NO: 2) |
| HQSSRLPHT; | (SEQ ID NO: 3) |
| SFAMH; | (SEQ ID NO: 4) |
| GFTFSSFAMH; | (SEQ ID NO: 5) |
| VIDTRGATYYADSVKG; | (SEQ ID NO: 6) |
| and | |
| LGNFYYGMDV; | (SEQ ID NO: 7) |

The scope of the present invention includes a pharmaceutical formulation comprising an anti-IGF1R antibody comprising a light chain variable region linked to a constant region, for example, a K chain and/or a heavy chain variable region linked to a constant region, for example a γ1, γ2, γ3 or γ4 constant region.

In an embodiment of the invention, the anti-IGF1R antibodies administered in the methods of the invention recognize human IGF1R, and/or sIGF1R (any soluble fragment of IGF1R); however, the methods of the present invention include administration of antibodies that recognize IGF1R from different species, for example, mammals (e.g., mouse, rat, rabbit, sheep or dog).

In an embodiment of the invention, an antibody or antigen-binding fragment thereof that binds “specifically” to IGF1R (e.g., human IGF1R) binds with a Kd of about 10⁻⁸ M or 10⁻⁷ M or a lower number; or, in an embodiment of the invention, with a Kd of about 1.28×10⁻¹⁰ M or a lower number by Biacore measurement or with a Kd of about 2.05×10⁻¹² or a lower number by KinExA measurement. In another embodiment of the invention, an antibody or antigen-binding fragment thereof that binds “specifically” to human IGF1R binds exclusively to human IGF1R and to no other protein at significant levels.

In an embodiment, the treatment methods comprise administration of an anti-IGF1R antibody of the invention, particularly an anti-IGF1R antibody that binds “specifically” to IGF1R, comprising one or more of the following characteristics:
(a) Binds to IGF1R with a K_{d} of about 86×10⁻¹¹ or a lower number;
(b) Has an off rate (K_{off}) for IGF1R of about 6.50×10⁻⁵ or a lower number;
(c) Has an on rate (Kₒₙ) for IGF1R of about 0.7×10⁵ or a higher number;
(d) Competes with IGF1 for binding to IGF1R;
(e) Inhibits autophosphorylation of IGF1R; and
(f) Inhibits anchorage-independent growth of a cell expressing IGF1R.

“K_{off}” refers to the off-rate constant for dissociation of the antibody from an antibody/antigen complex.

“Kₒₙ” refers to the rate at which the antibody associates with the antigen.

“K_{d}” refers to the dissociation constant of a particular antibody/antigen interaction.

K_{d}=K_{off}/Kₒₙ.

The term “monoclonal antibody,” as used herein, includes an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modifier “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method first described by Kohler, et al., (1975) Nature 256: 495 or other methods known in the art.

A polyclonal antibody is an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai, et al., (1990) Clin. Exp. Immunol. 79: 315-321, Kostelny, et al., (1992) J Immunol. 148:1547-1553. In addition, bispecific antibodies may be formed as “diabodies” (Holliger, et al., (1993) PNAS USA 90:6444-6448) or as “Janusins” (Traunecker, et al., (1991) EMBO J. 10:3655-3659 and Traunecker, et al., (1992) Int. J. Cancer Suppl. 7:51-52).

The term “fully human antibody” refers to an antibody which comprises human immunoglobulin amino acid sequences only. A fully human antibody may contain murine carbohydrate chains if produced in a mouse, in a mouse cell or in a hybridoma derived from a mouse cell. Similarly, “mouse antibody” refers to an antibody which comprises mouse immunoglobulin sequences only.

The present invention includes administration of “chimeric antibodies”—an antibody which comprises a variable region of one species fused or chimerized with an antibody region (e.g., constant region) from another species (e.g., mouse, horse, rabbit, dog, cow, chicken). These antibodies may be used to modulate the expression or activity of IGF1R in the non-human species.

“Single-chain Fv” or “sFv” antibody fragments have the V_{H} and V_{L} domains of an antibody, wherein these domains are present in a single polypeptide chain. Generally, the sFv polypeptide further comprises a polypeptide linker between the V_{H} and V_{L} domains which enables the sFv to form the desired structure for antigen binding. Techniques described for the production of single chain antibodies (U.S. Pat. Nos. 5,476,786; 5,132,405 and 4,946,778) can be adapted to produce anti-IGF1R-specific single chain antibodies. For a review of sFv see Pluckthun in The Pharmacology of Monoclonal Antibodies, vol. 113, Rosenburg and Moore eds. Springer-Verlag, N.Y., pp. 269-315 (1994).

“Disulfide stabilized Fv fragments” and “dsFv” refer to antibody molecules comprising a variable heavy chain (V_{H}) and a variable light chain (V_{L}) which are linked by a disulfide bridge.

Antibody fragments for use in the formulations administered in the methods of the present invention also include F(ab)₂ fragments which may be produced by enzymatic cleavage of an IgG by, for example, pepsin. Fab fragments may be produced by, for example, reduction of F(ab)₂ with dithiothreitol or mercaptoethylamine. A Fab fragment is a V_{L}-C_{L} chain appended to a V_{H}-C_{H1} chain by a disulfide bridge. A F(ab)₂ fragment is two Fab fragments which, in turn, are appended by two disulfide bridges. The Fab portion of an F(ab)₂ molecule includes a portion of the F_{c} region between which disulfide bridges are located.

An F_{V} fragment is a V_{L} or V_{H} region.

Depending on the amino acid sequences of the constant domain of their heavy chains, immunoglobulins can be assigned to different classes. There are at least five major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2.

The anti-IGF1R antibodies of the formulations used in the invention may also be conjugated to a chemical moiety. The chemical moiety may be, inter alia, a polymer, a radionuclide or a cytotoxic factor. In an embodiment of the invention, the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antibody fragments of the formulations administered in the methods of the invention may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The antibodies and antibody fragments of the formulations used in the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibodies and antibody fragments of the formulations administered in the methods of the present invention can also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, momordica charantia inhibitor, curcin, crotin, saponaria officinalis inhibitor, mitogellin, restrictocin, phenomycin, and enomycin.

Any method known in the art for conjugating the antibodies and antibody fragments of the formulations used in the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

In a preferred formulation for use in the claimed methods, 15H12/19D12 LC, LCA, LCB, LCC, LCD, LCE or LCF is dimerized with any other immunoglobulin heavy chain, for example, any immunoglobulin heavy chain set forth herein. Likewise, in an embodiment, 15H12/19D12 HC, HCA or HCB is dimerized with any light chain, for example, any light chain set forth herein. For example, 15H12/19D12 HCA or HCB can be dimerized with 15H12/19D12 LCC, LCD, LCE or LCF. In an embodiment, the light immunoglobulin chain and or the heavy immunoglobulin chain of an anti-IGF1R antibody of the invention is a mature chain.

Antibody chains are shown below. Dotted underscored type encodes the signal peptide. Solid underscored type encodes the CDRs. Plain type encodes the framework regions. Antibody chains are mature fragments which lack the signal peptide.

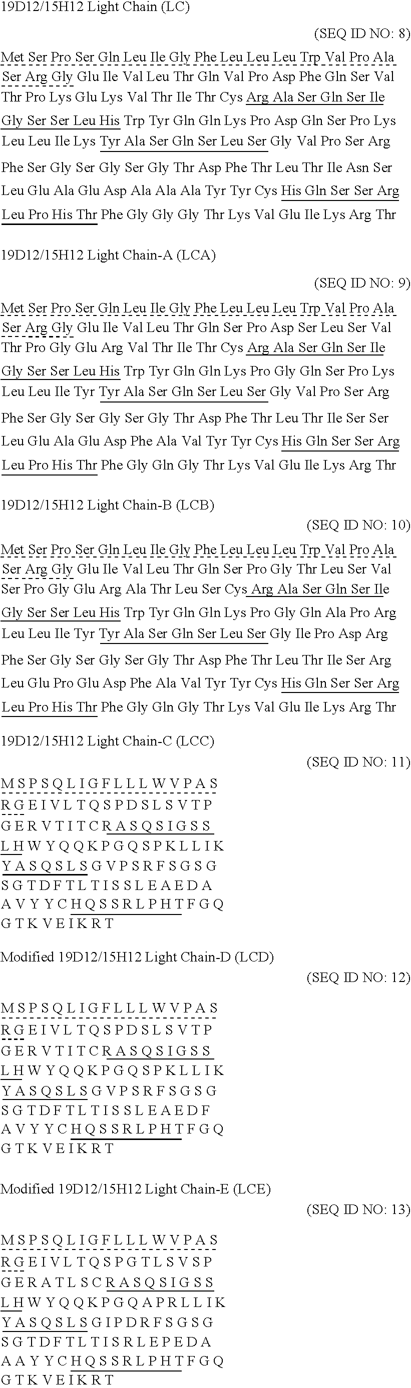

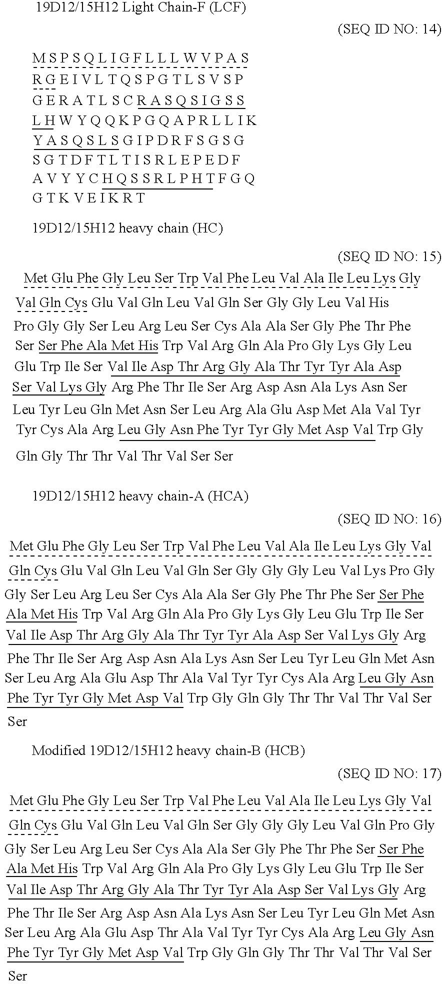

Antibodies including, for example, light chain F may be designated LCF and antibodies including heavy chain A may be designated HCA. Antibodies including light chain F and heavy chain A may be designated LCF/HCA.

Cell lines containing plasmids encoding the above-referenced antibody chains were deposited at the American Type Culture Collection as follows:
(i) CMV promoter-15H12/19D12 HCA (γ4)-

Deposit name: “15H12/19D12 HCA (γ4)”;

ATCC accession No.: PTA-5214;
(ii) CMV promoter-15H12/19D12 HCB (γ4)-

Deposit name: “15H12/19D12 HCB (γ4)”;

ATCC accession No.: PTA-5215;
(iii) CMV promoter-15H12/19D12 HCA (γ1)-

Deposit name: “15H12/19D12 HCA (γ1)”;

ATCC accession No.: PTA-5216;
(iv) CMV promoter-15H12/19D12 LCC (κ)-

Deposit name: “15H12/19D12 LCC (κ)”;

ATCC accession No.: PTA-5217;
(v) CMV promoter-15H12/19D12 LCD (κ)-

Deposit name: “15H12/19D12 LCD (κ)”;

ATCC accession No.: PTA-5218;
(vi) CMV promoter-15H12/19D12 LCE (κ)-

Deposit name: “15H12/19D12 LCE (κ)”;

ATCC accession No.: PTA-5219; and
(vii) CMV promoter-15H12/19D12 LCF (κ)-

Deposit name: “15H12/19D12 LCF (κ)”;

ATCC accession No.: PTA-5220;

HCA is heavy chain A; HCB is heavy chain B, LCC is light chain C; LCD is light chain D; LCE is light chain E and LCF is light chain F.

The above-identified plasmids were deposited, under the Budapest Treaty, on May 21, 2003 with the American Type Culture Collection (ATCC); 10801 University Boulevard; Manassas, Va. 20110-2209. All restrictions on access to the plasmids deposited in ATCC will be removed upon grant of a patent (see published U.S. patent application no. US2004/0018191).

The present application comprises methods for treating or preventing a medical condition comprising administering to a subject a therapeutically effective amount of a formulation as set forth herein, wherein the formulation comprises antibodies and antigen-binding fragments thereof whose immunoglobulin chains (e.g., mature chains thereof), for example, heavy chains or light chains, which are encoded by the inserts in the plasmids in the cell lines deposited at the ATCC as described above. Formulations comprising immunoglobulins encoded by the plasmids comprising a different constant region than that indicated above may also be used in the methods of the present invention.

### Further Therapeutic Agents and Procedures

In an embodiment of the invention, a further chemotherapeutic agent is provided and/or administered in association with the anti-IGF1R formulation of the invention. In an embodiment, the further chemotherapeutic agent is a platinum-based compound, a signal transduction inhibitor, a cell cycle inhibitor, a IGF/IGF1R system modulator (e.g., inhibitors or activators), a farnesyl protein transferase (FPT) inhibitor, an epidermal growth factor receptor (EGFR) inhibitor, a HER2 inhibitor, a vascular epidermal growth factor (VEGF) receptor inhibitor, a mitogen activated protein (MAP) kinase inhibitor, a MEK inhibitor, an AKT inhibitor, a mTOR inhibitor, a pl3 kinase inhibitor, a Raf inhibitor, a cyclin dependent kinase (CDK) inhibitor, a microtubule stabilizer, a microtubule inhibitor, a SERMs/Antiestrogen, an aromatase inhibitor, an anthracycline, a proteasome inhibitor or an agent which inhibits insulin-like growth factor (IGF) production.

The methods of the invention include administration of an anti-IGF1R formulation “in association with” one or more further therapeutic agents or procedures. The term “in association with” indicates that the components (e.g., anti-IGF1R antibody along with paclitaxel) can be formulated into a single composition for simultaneous delivery or formulated separately into two or more compositions (e.g., a kit). Furthermore, each component can be administered to a subject at a different time than when the other component is administered; for example, each administration may be given non-simultaneously (e.g., separately or sequentially) at several intervals over a given period of time. Moreover, the separate components may be administered to a subject by the same or by a different route (e.g., wherein an anti-IGF1R antibody formulation is administered parenterally and gefitinib is administered orally).

In an embodiment of the invention, the anti-IGF1R formulation of the invention is provided and/or administered in association with a farnesyl protein transferase (FPT) inhibitor including tricyclic amide compounds such as any of those disclosed in U.S. Pat. No. 5,719,148 or in U.S. Pat. No. 5,874,442. In an embodiment, the anti-IGF1R formulation of the invention is provided in association with any compound represented by the following formula:

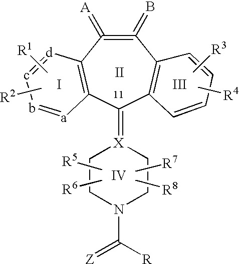

or a pharmaceutically acceptable salt or solvate thereof, wherein: one of a, b, c and d represents N or NR⁹ wherein R⁹ is O⁻, —CH₃ or —(CH₂)ₙCO₂H wherein n is 1 to 3, and the remaining a, b, c and d groups represent CR¹ or CR²; or each of a, b, c, and d are independently selected from CR¹ or CR²; each R¹ and each R² is independently selected from H, halo, —CF₃, —OR¹⁰, —COR¹⁰, —SR¹⁰, —S(O)ₜR¹¹ (wherein t is 0, 1 or 2), —SCN, —N(R¹⁰)₂, —NO₂, —OC(O)R¹⁰, —CO₂R¹⁰, —OCO₂R¹¹, —CN, —NHC(O)R¹⁰, —NHSO₂R¹⁰, —CONHR¹⁰, —CONHCH₂CH₂OH, —NR¹⁰COOR¹¹, —SR¹¹C(O)OR¹¹,

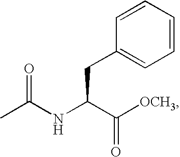

—SR¹¹N(R⁷⁵)₂ (wherein each R⁷⁵ is independently selected from H and —C(O)OR¹¹), benzotriazol-1-yloxy, tetrazol-5-ylthio, or substituted tetrazol-5-ylthio, alkynyl, alkenyl or alkyl, said alkyl or alkenyl group optionally being substituted with halo, —OR¹⁰ or —CO₂R¹⁰; R³ and R⁴ are the same or different and each independently represents H, any of the substituents of R¹ and R², or R³ and R⁴ taken together represent a saturated or unsaturated C₅-C₇ fused ring to the benzene ring; R⁵, R⁶, R⁷ and R⁸ each independently represents H, —CF₃, —COR¹⁰, alkyl or aryl, said alkyl or aryl optionally being substituted with —OR¹⁰, —SR¹⁰, —S(O)ₜR¹¹, —NR¹⁰COOR¹¹, —N(R¹⁰)₂, —NO₂, —COR¹⁰, —OCOR¹⁰, —OCO₂R¹¹, —CO₂R¹⁰, OPO₃R¹⁰ or one of R⁵, R⁶, R⁷ and R⁸ can be taken in combination with R⁴⁰ as defined below to represent —(CH₂)ᵣ— wherein r is 1 to 4 which can be substituted with lower alkyl, lower alkoxy, —CF₃ or aryl, or R⁵ is combined with R⁶ to represent ═O or ═S and/or R⁷ is combined with R⁸ to represent ═O or ═S; R¹⁰ represents H, alkyl, aryl, or aralkyl; R¹¹ represents alkyl or aryl; X represents N, CH or C, which C may contain an optional double bond, represented by the dotted line, to carbon atom 11; the dotted line between carbon atoms 5 and 6 represents an optional double bond, such that when a double bond is present, A and B independently represent —R¹⁰, halo, —OR¹¹, —OCO₂R¹¹ or —OC(O)R¹⁰, and when no double bond is present between carbon atoms 5 and 6, A and B each independently represent H₂, —(OR¹¹)₂; H and halo, dihalo, alkyl and H, (alkyl)₂, —H and —OC(O)R¹⁰, H and —OR¹⁰, ═O, aryl and H, ═NOR¹⁰ or —O—(CH₂)ₚ—O— wherein p is 2, 3 or 4; R represents R⁴⁰, R⁴², R⁴⁴, or R⁵⁴, as defined below; R⁴⁰ represents H, aryl, alkyl, cycloalkyl, alkenyl, alkynyl or -D wherein -D represents

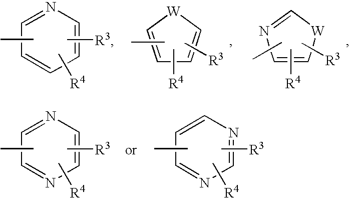

wherein R³ and R⁴ are as previously defined and W is O, S or NR¹⁰ wherein R¹⁰ is as defined above; said R⁴⁰ cycloalkyl, alkenyl and alkynyl groups being optionally substituted with from 1-3 groups selected from halo, —CON(R¹⁰)₂, aryl, —CO₂R¹⁰, —OR¹², —SR¹², —N(R¹⁰)₂, —N(R¹⁰)CO₂R¹¹, —COR¹², —NO₂ or D, wherein -D, R¹⁰ and R¹¹ are as defined above and R¹² represents R¹⁰, —(CH₂)ₘOR¹⁰ or —(CH₂)_{q}CO₂R¹⁰ wherein R¹⁰ is as previously defined, m is 1 to 4 and q is 0 to 4; said alkenyl and alkynyl R⁴⁰ groups not containing —OH, —SH or —N(R¹⁰)₂ on a carbon containing a double or triple bond respectively; or R⁴⁰ represents phenyl substituted with a group selected from —SO₂NH₂, —NHSO₂CH₃, —SO₂NHCH₃, —SO₂CH₃, —SOCH₃, —SCH₃, or —NHSO₂CF₃, preferably, said group is located in the para position of the phenyl ring; or
R⁴⁰ represents a group selected from

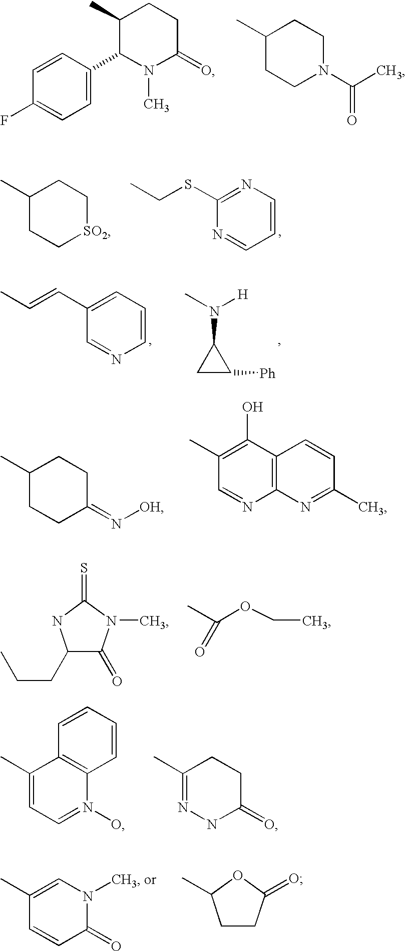

R⁴² represents

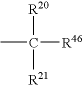

wherein R²⁰, R²¹ and R⁴⁶ are each independently selected from the group consisting of:

(1) H;

(2) —(CH₂)_{q}SC(O)CH₃ wherein q is 1 to 3;

(3) —(CH₂)_{q}OSO₂CH₃ wherein q is 1 to 3;

(4) —OH;

(5) —CS(CH₂)_{w}(substituted phenyl) wherein w is 1 to 3 and the substitutents on said substituted phenyl group are the same substitutents as described below for said substituted phenyl;

(6) —NH₂;

(7) —NHCBZ;

(8) —NHC(O)OR²² wherein R²² is an alkyl group having from 1 to 5 carbon atoms, or R²² represents phenyl substituted with 1 to 3 alkyl groups;

(9) alkyl;

(10) —(CH₂)ₖphenyl wherein k is 1 to 6;

(11) phenyl;

(12) substituted phenyl wherein the substituents are selected from the group consisting of: halo, NO₂, —OH, —OCH₃, —NH₂, —NHR²², —N(R²²)₂, alkyl, —O(CH₂)ₜphenyl (wherein t is from 1 to 3), and —O(CH₂)ₜsubstituted phenyl (wherein t is from 1 to 3);

(13) naphthyl;

(14) substituted naphthyl, wherein the substituents are as defined for substituted phenyl above;

(15) bridged polycyclic hydrocarbons having from 5 to 10 carbon atoms;

(16) cycloalkyl having from 5 to 7 carbon atoms;

(17) heteroaryl;

(18) hydroxyalkyl;

(19) substituted pyridyl or substituted pyridyl N-oxide wherein the substituents are selected from methylpyridyl, morpholinyl, imidazolyl, 1-piperidinyl, 1-(4-methylpiperazinyl), —S(O)ₜR¹¹, or any of the substituents given above for said substituted phenyl, and said substitutents are bound to a ring carbon by replacement of the hydrogen bound to said carbon;

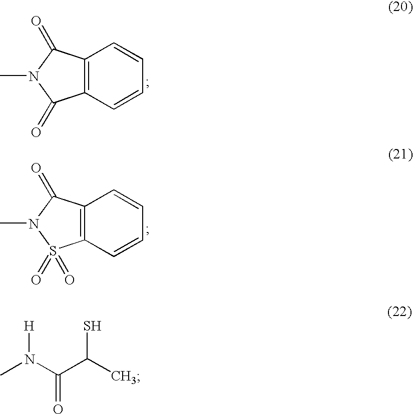

(23) —NHC(O)—(CH₂)ₖ-phenyl or —NH(O)—(CH₂)ₖ-substituted phenyl, wherein said k is as defined above;

(24) piperidine Ring V:

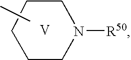

wherein R⁵⁰ represents H, alkyl, alkylcarbonyl, alkyloxycarbonyl, haloalkyl, or —C(O)NH(R¹⁰) wherein R¹⁰ is H or alkyl; Ring V includes

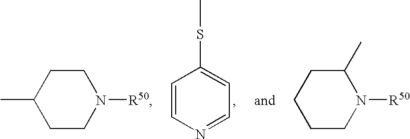

examples of Ring V include:

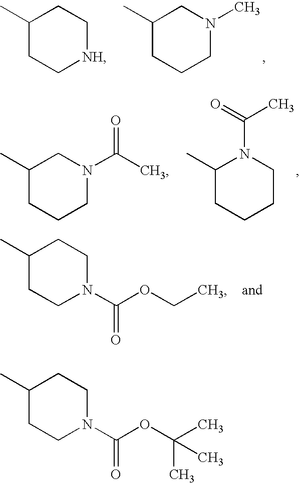

(25) —NHC(O)CH₂C₆H₅ or —NHC(O)CH₂-substituted-C₆H₅;

(26) —NHC(O)OC₆H₅;

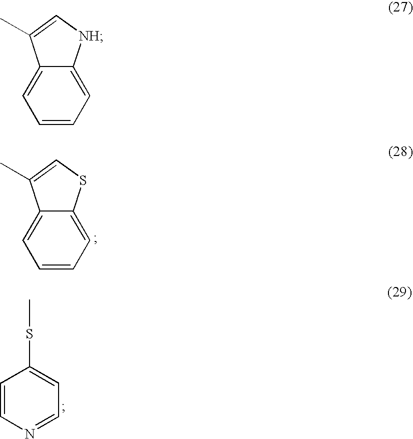

(30) —OC(O)-heteroaryl, for example

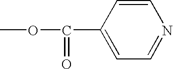

(31) —O-alkyl (e.g., —OCH₃); and

(32) —CF₃;

(33) —CN;

(34) a heterocycloalkyl group of the formula

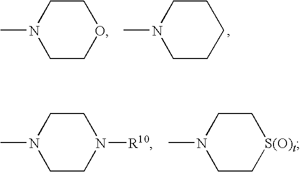

and

(35) a piperidinyl group of the formula

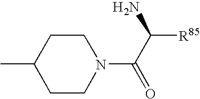

wherein R⁸⁵ is H, alkyl, or alkyl substituted by —OH or —SCH₃; or R²⁰ and R²¹ taken together form a ═O group and the remaining R⁴⁶ is as defined above; or

Two of R²⁰, R²¹ and R⁴⁶ taken together form piperidine Ring V

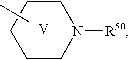

wherein R50 represents H, alkyl (e.g., methyl), alkylcarbonyl (e.g., CH3C(O)—), alkyloxycarbonyl (e.g., —C(O)O-t-C4H9, —C(O)OC2H5, and —C(O)OCH3), haloalkyl (e.g., trifluro-methyl), or —C(O)NH(R10) wherein R10 is H or alkyl; Ring V includes

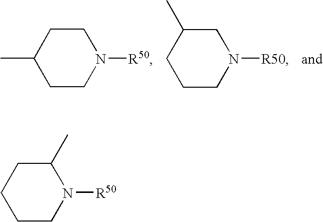

examples of Ring V include:

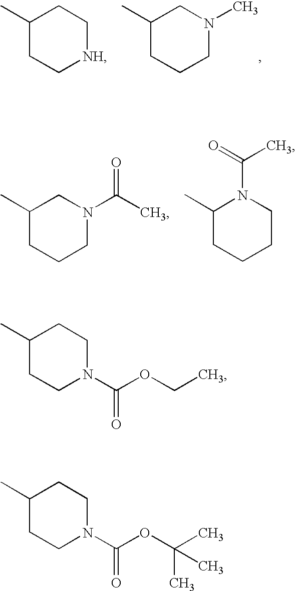

with the proviso R46, R20, and R21 are selected such that the carbon atom to which they are bound does not contain more than one heteroatom (i.e., R46, R20, and R21 are selected such that the carbon atom to which they are bound contains 0 or 1 heteroatom); R⁴⁴ represents

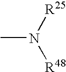

wherein R²⁵ represents heteroaryl, N-methylpiperdinyl or aryl; and R⁴⁸ represents H or alkyl; R⁵⁴ represents an N-oxide heterocyclic group of the formula (i), (ii), (iii) or (iv):

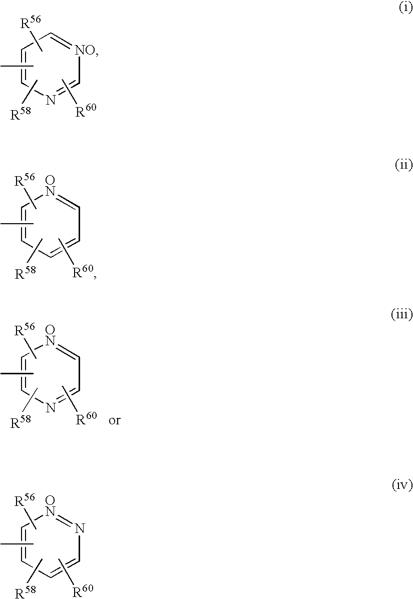

wherein R⁵⁶, R⁵⁸, and R⁶⁰ are the same or different and each is independently selected from H, halo, —CF₃, —OR¹⁰, —C(O)R¹⁰, —SR¹⁰, —S(O)ₑR¹¹ (wherein e is 1 or 2), —N(R¹⁰)₂, —NO₂, —CO₂R¹⁰, —OCO₂R¹¹, —OCOR¹⁰, alkyl, aryl, alkenyl or alkynyl, which alkyl may be substituted with —OR¹⁰, —SR¹⁰ or —N(R¹⁰)₂ and which alkenyl may be substituted with OR¹¹ or SR¹¹; or R⁵⁴ represents an N-oxide heterocyclic group of the formula (ia), (iia), (iiia) or (iva):

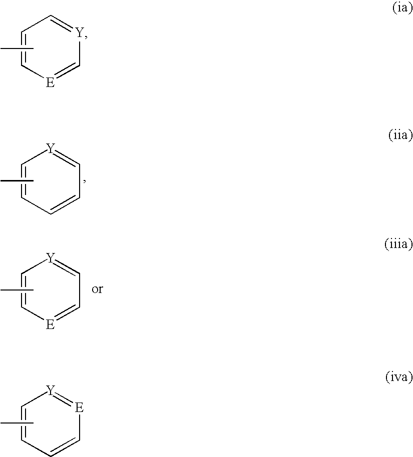

wherein Y represents N⁺—O⁻ and E represents N; or

R⁵⁴ represents an alkyl group substituted with one of said N-oxide heterocyclic groups (i), (ii), (iii), (iv), (ia), (iia), (iiia) or (iva); Z represents O or S such that R can be taken in combination with R⁵, R⁶, R⁷ or R⁸ as defined above, or R represents R⁴⁰, R⁴², R⁴⁴ or R⁵⁴. Examples of R20, R21, and R46 for the above formulas include:

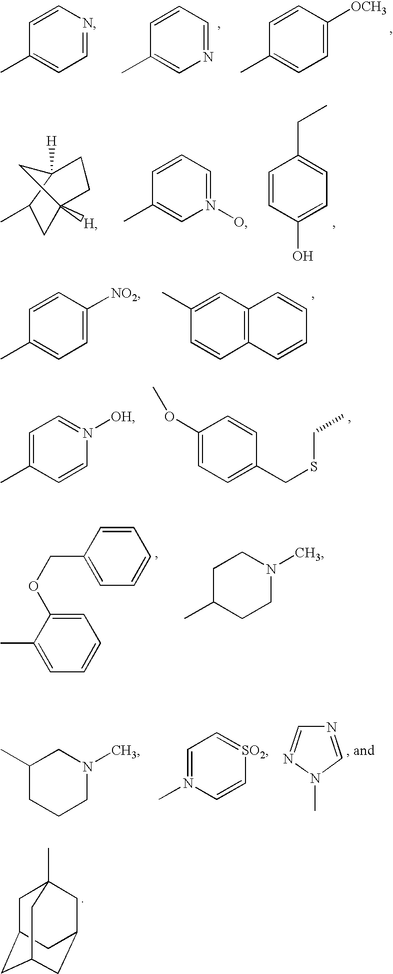

Examples of R25 groups include:

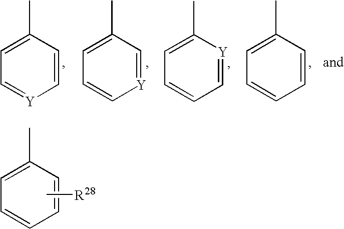

wherein Y represents N or NO, R28 is selected from the group consisting of: C1 to C4 alkyl, halo, hydroxy, NO2, amino (—NH2), —NHR30, and —N(R30)2 wherein R30 represents C1 to C6 alkyl.

In one embodiment, the following tricyclic amide is provided and/or administered in association with the anti-IGF1R formulation of the invention:

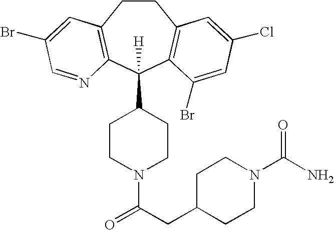

(lonafarnib; Sarasar™; Schering-Plough; Kenilworth, N.J.). In another embodiment, one of the following FPT inhibitors is provided and/or administered in association with the anti-IGF1R formulation in the methods of the invention:

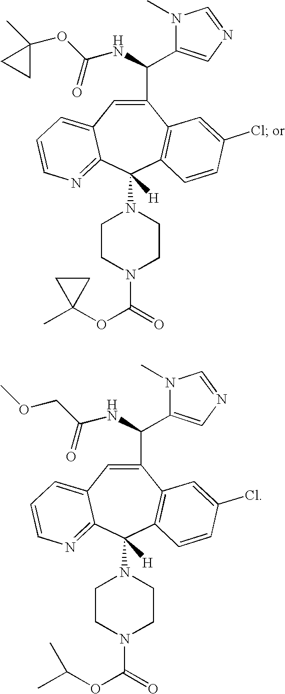

An FPT inhibitor, which, in an embodiment, is provided and/or administered in association with the anti-IGF1R formulation of the invention, includes BMS-214662

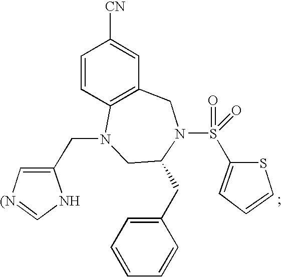

Hunt at al., J. Med. Chem. 43(20):3587-95 (2000); Dancey et al., Curr. Pharm. Des. 8:2259-2267 (2002); (R)-7-cyano-2,3,4,5-tetrahydro-1-(1H-imidazol-4-ylmethyl)-3-(phenylmethyl)-4-(2-thienylsulfonyl)-1H-1,4-benzodiazepine)) and R155777 (tipifarnib; Garner et al., Drug Metab. Dispos. 30(7):823-30 (2002); Dancey et al., Curr. Pharm. Des. 8:2259-2267 (2002); (B)-6-[amino(4-chlorophenyl)(1-methyl-1H-imidazol-5-yI)-methyl]-4-(3-chlorophenyl)-1-methyl-2(1H)-quinolinonej

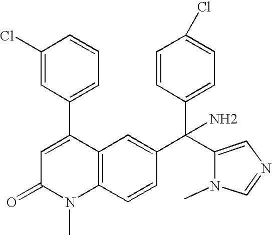

sold as Zarnestra™; Johnson & Johnson; New Brunswick, N.J.).

In an embodiment, an inhibitor which antagonizes the action of the EGF Receptor or HER2, is provided and/or administered in association with the anti-IGF1R formulation in the methods of the invention: for example, HuMax-CD20 (sold by Genmab; Copenhagen, Denmark); Campath-1H® (Riechmann et al., Nature 332:323 (1988)); HuMax-EGFr (sold by Genmab; Copenhagen, Denmark); pertuzumab (Omnitarg™, 2C4; Genentech; San Francisco, Calif.); bevacizumab (Presta et al., Cancer Res 57:4593-9 (1997); sold as Avastin® by Genentech; San Francisco, Calif.); Ibritumomab tiuxetan (sold as Zevalin® by Biogen Idec; Cambridge, Mass.); Tositumomab and Iodine I¹³¹ (sold as Bexxar® by Corixa Corp.; Seattle, Wash. and Glaxosmithkline; Philadelphia, Pa.); gemtuzumab ozogamicin (sold as Mylotarg® by Wyeth Ayerst; Madison, N.J.) or MDX-010 (Medarex; Princeton, N.J.); trastuzumab (sold as Herceptin®; Genentech, Inc.; S. San Francisco, Calif.); CP-724714

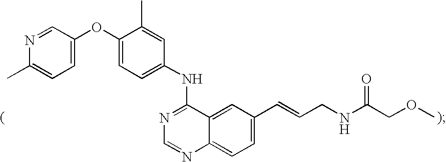

### TAK-165

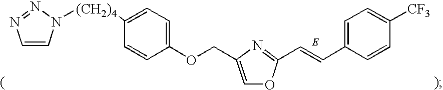

### HKI-272

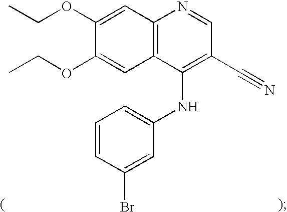

gefitinib (Baselga et al., Drugs 60 Suppl 1:33-40 (2000); ZD-1893; 4-(3-chloro-4-fluoroanhlino)-7-methoxy-6-(3-morphollnopropoxy) quinazoline;
sold as Iressa AstraZeneca; Wilmington, Del.;

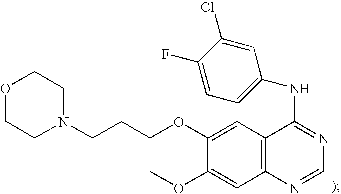

### OSI-774

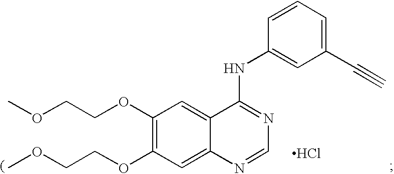

erlotinib, Hidalgo et al., J. Clin. Oncol. 19(13): 3267-3279 (2001)), Lapatanib

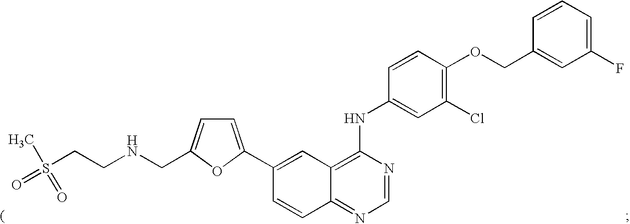

GW2016; Rusnak et al., Molecular Cancer Therapeutics 1:85-94 (2001); N-{3-Chloro-4-[(3-fluorobenzyl)oxy]phenyl}-6-[5-({[2-(methylsulfonyl )ethyl]amino}methyl)-2-furyl]-4-quinazolinamine; PCT Application No. W099/35146), Canertinib (Cl-1033;

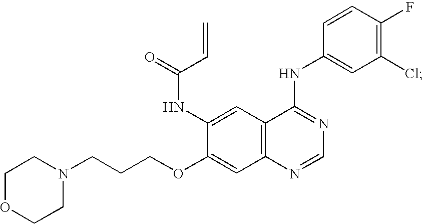

Erlichman et al., Cancer Res. 61 (2):739-48 (2001); Smaill etal., J. Med. Chem. 43(7):1380-97 (2000)), ABX-EGF antibody (Abgenix, Inc.; Freemont, Calif.; Yang et al., Cancer Res. 59(6):1236-43 (1999); Yang et al., Crit Rev Oncol Hematol. 38(1):17-23 (2001)), erbitux (U.S. Pat. No. 6,217,866; IMC-C225, cetuximab; lmclone; New York, N.Y.), EKB-569

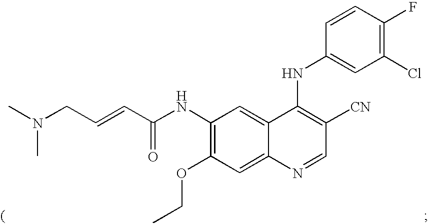

Wissner et al., J. Med. Chem. 46(1): 49-63 (2003)), PKI-166

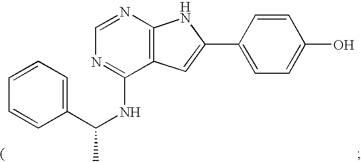

CGP-75166), GW-572016, any anti-EGFR antibody and any anti-HER2 antibody.

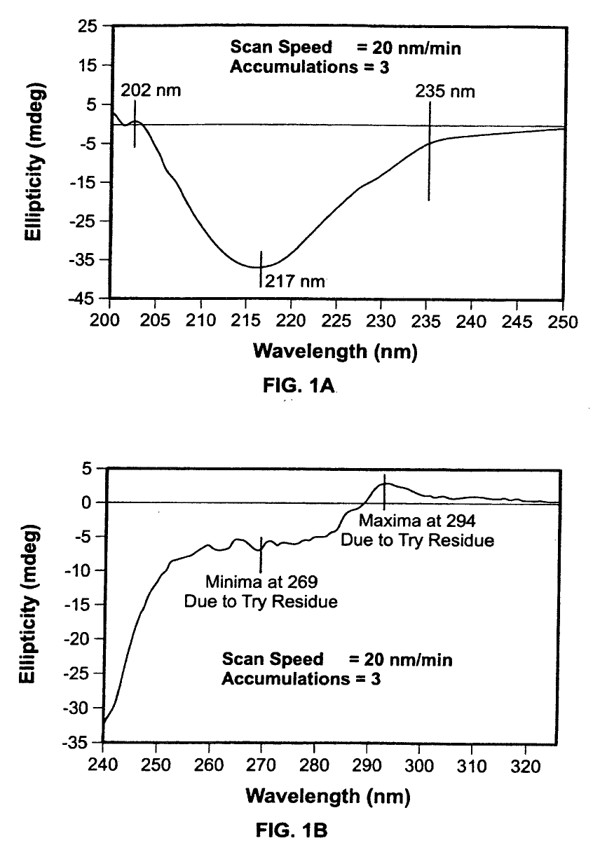

One or more of numerous other small molecules, which have been described as being useful to inhibit EGFR, are, in a embodiment of the invention, may be provided and/or administered in association with the anti-IGF1R formulation of the invention. For example, U.S. Pat. No. 5,656,655, discloses styryl substituted heteroaryl compounds that inhibit EGFR. U.S. Pat. No. 5,646,153 discloses bis mono and/or bicyclic aryl heteroaryl carbocyclic and heterocarbocyclic compounds that inhibit EGFR and/or PDGFR. U.S. Pat. No. 5,679,683 discloses tricyclic pyrimidine compounds that inhibit the EGFR. U.S. Pat. No. 5,616,582 discloses quinazoline derivatives that have receptor tyrosine kinase inhibitory activity. Fry et al., Science 265 1093-1095 (1994) discloses a compound having a structure that inhibits EGFR (see FIG. 1 of Fry et al.). U.S. Pat. No. 5,196,446, discloses heteroarylethenediyl or heteroarylethenediylaryl compounds that inhibit EGFR. Panek, et al., Journal of Pharmacology and Experimental Therapeutics 283, 1433-1444 (1997) disclose a compound identified as PD166285 that inhibits the EGFR, PDGFR, and FGFR families of receptors. PD166285 is identified as 6-(2,6-dichlorophenyl)-2-(4-(2-diethylaminoethoxy)phenylamino)-8-methyl-8H-pyrido(2,3-d)pyrimidin-7-one.

In an embodiment of the invention, the anti-IGF1R formulation of the invention is provided and/or administered in association with a LHRH (Lutenizing hormone-releasing hormone) agonist such as the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4];

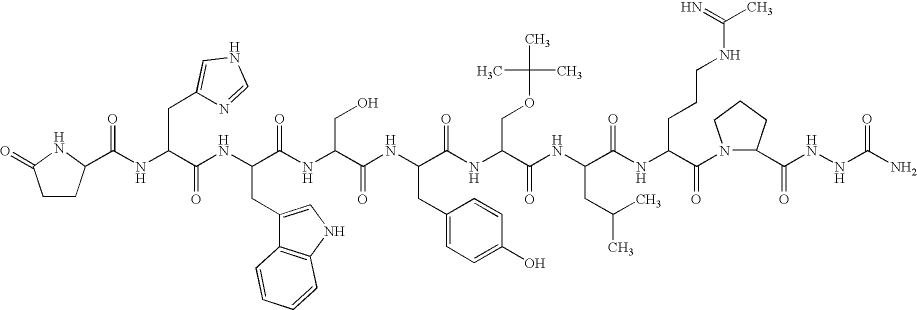

(goserelin acetate; sold as Zoladex® by AstraZeneca UK Limited; Macclesfield, England),

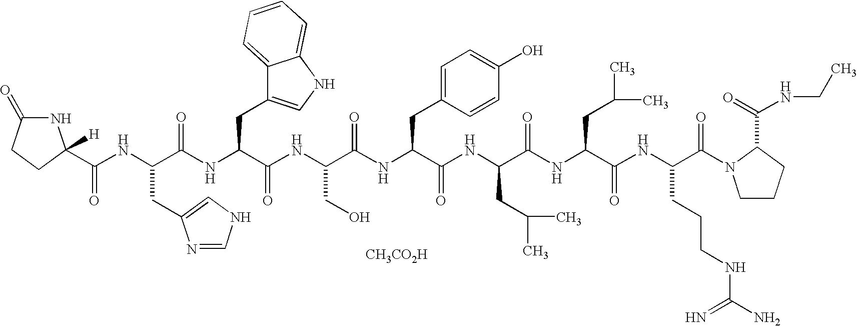

(leuprolide acetate; sold as Eligard® by Sanofi-Synthelabo Inc.; New York, N.Y.) or

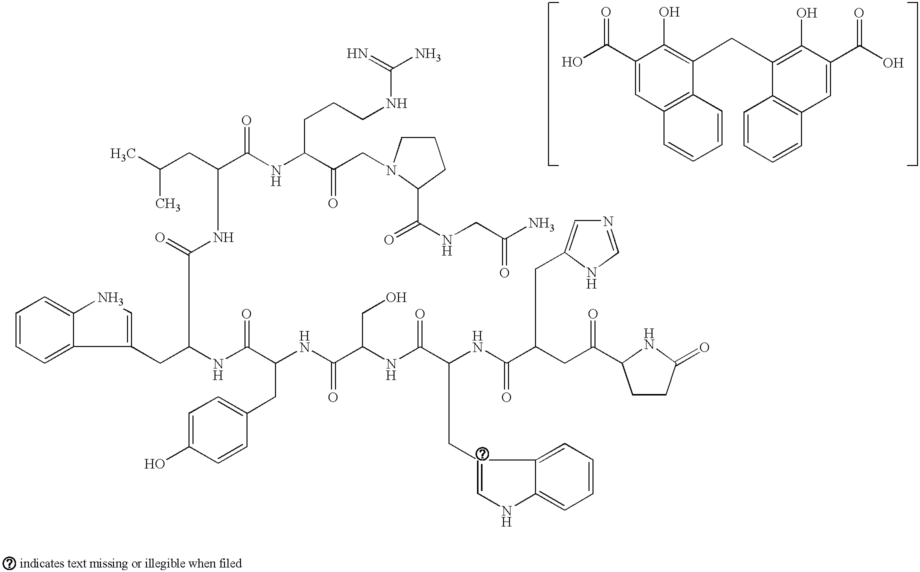

(triptorelin pamoate; sold as Trelstar® by Pharmacia Company, Kalamazoo, Mich.).

In an embodiment of the invention, the anti-IGF1R formulation of the invention is provided and/or administered in association with the FOLFOX regimen (oxaliplatin

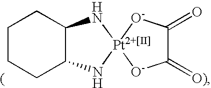

together with infusional fluorouracil

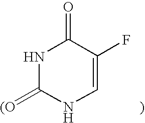

and folinic acid

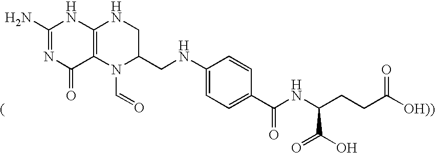

(Chaouche et al., Am. J. Clin. Oncol. 23(3):288-289 (2000); de Gramont et al., J. Clin. Oncol. 18(16):2938-2947 (2000)).

In an embodiment of the invention, the anti-IGF1R formulation of the invention is provided and/or administered in association with 5′-deoxy-5-fluorouridine

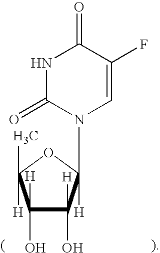

In an embodiment of the invention, the anti-IGF1R formulation of the invention is provided and/or administered in association with Asparaginase; Bacillus Calmette-Guerin (BCG) vaccine (Garrido et al., Cytobios. 90(360):47-65 (1997));

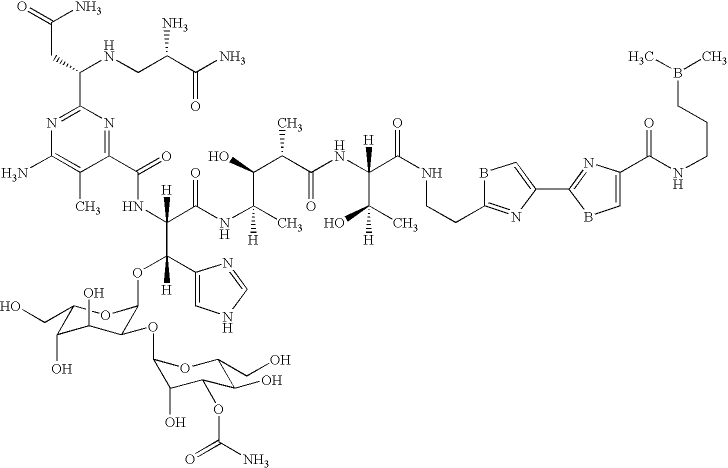

### (Bleomycin);

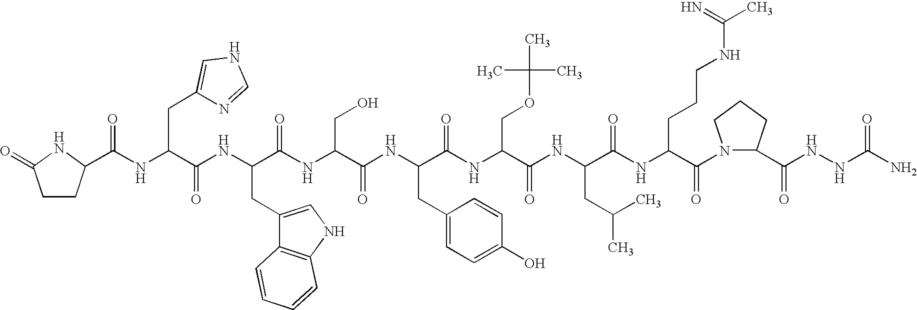

### (Buserelin); or

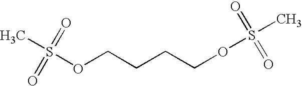

(Busulfan; 1,4-butanediol, dimethanesulfonate; sold as Busulfex® by ESP Pharma, Inc.; Edison, New Jersey).

In an embodiment of the invention, a platinum-based anti-cancer compound, such as oxaliplatin

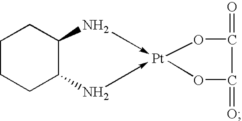

sold as Eloxatin™ by Sanofi-Synthelabo Inc.; New York, N.Y.),

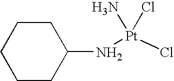

### (JM118),

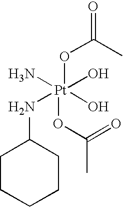

### (JM383),

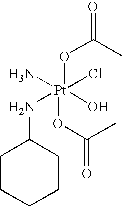

### (JM559),

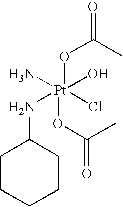

### (JM518),

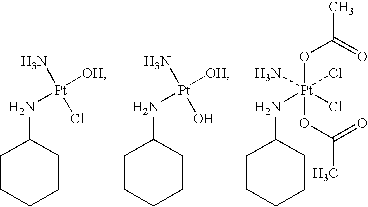

(satraplatin) or

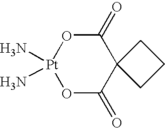

(carboplatin) is provided and/or administered in association with the anti-IGF1R formulation of the invention.

In an embodiment of the invention, the anti-IGF1R formulation of the invention is provided and/or administered in association with DES (diethylstilbestrol;

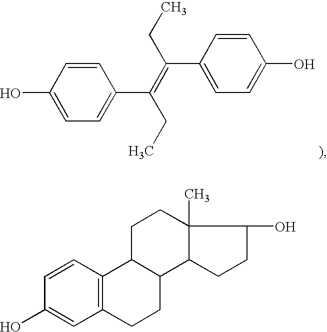

(estradioll sold as Estrol® by Warner Chilcott, Inc.; Rockaway, N.J.) or conjugated estrogens (sold as Premarin® by Wyeth Pharmaceuticals Inc.; Philadelphia, Pa.).

In an embodiment of the invention, the anti-IGF1R formulation of the invention is provided and/or administered in association with

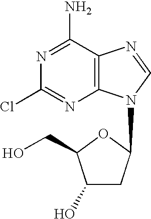

### (Cladribine);

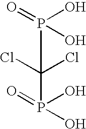

### (Clodronate);

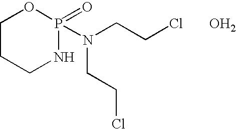

### (Cyclophosphamide);

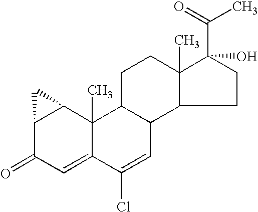

### (Cyproterone);

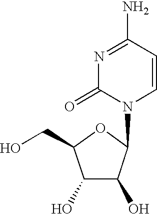

### (Cytarabine);

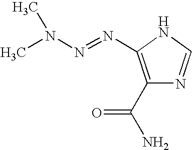

### (Dacarbazine);

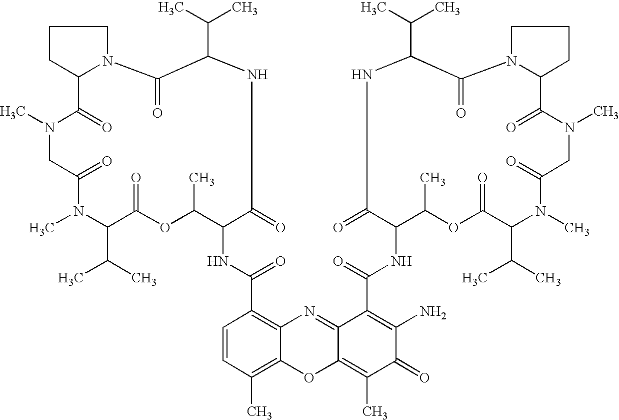

### (Dactinomycin);

In an embodiment of the invention, a VEGF receptor inhibitor, for example, PTK787/ZK 222584 (Thomas et al., Semin Oncol. 30(3 Suppl 6):32-8 (2003)) or the humanized anti-VEGF antibody Bevacizumab (sold under the brand name Avastin™; Genentech, Inc.; South San Francisco, Calif.) is provided and/or administered in association with the anti-IGF1R formulation of the invention.

In an embodiment of the invention, a MAP kinase inhibitor, for example, VX-745 (Haddad, Curr Opin. Investig. Drugs 2(8):1070-6 (2001)), is provided and/or administered in association with the anti-IGF1R formulation of the invention.

In an embodiment of the invention, a MAP kinase kinase (MEK) inhibitor, such as PD 184352 (Sebolt-Leopold, et al. Nature Med. 5: 810-816 (1999)), is provided and/or administered in association with the anti-IGF1R formulation of the invention.

In an embodiment of the invention, an mTOR inhibitor such as rapamycin or CCI-779 (Sehgal et al., Med. Res. Rev., 14:1-22 (1994); Elit, Curr. Opin. Investig. Drugs 3(8):1249-53 (2002)) is provided and/or administered in association with the anti-IGF1R formulation of the invention.

In an embodiment of the invention, a pl3 kinase inhibitor, such as LY294002, LY292223, LY292696, LY293684, LY293646 (Vlahos et al., J. Biol. Chem. 269(7): 5241-5248 (1994)) or wortmannin is provided and/or administered in association with the anti-IGF1R formulation of the invention.

In an embodiment of the invention, a Raf inhibitor, such as BAY-43-9006

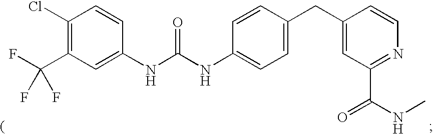

Wilhelm et al., Curr. Pharm. Des. 8:2255-2257 (2002)), ZM336372, L-779,450 or any other Raf inhibitor disclosed in Lowinger et al., Curr. Pharm Des. 8:2269-2278 (2002) is provided and/or administered in association with the anti-IGF1R formulation of the invention.

In an embodiment of the invention, a cyclin dependent kinase inhibitor, such as flavopiridol (L86-8275/HMR 1275; Senderowicz, Oncogene 19(56): 6600-6606 (2000)) or UCN-01 (7-hydroxy staurosporine; Senderowicz, Oncogene 19(56): 6600-6606 (2000)), is provided and/or administered in association with the anti-IGF1R formulation of the invention.

In an embodiment of the invention, an IGF/IGFR inhibitor, such as an IGF inhibitory peptide (see e.g., U.S. Published Patent Application No. 20030092631 A1; PCT Application Publication NOs. WO 03/27246 A2; WO 02/72780) or any 4-amino-5-phenyl-7-cyclobutyl-pyrrolo[2,3-d]pyrimidine derivative, such as those disclosed in PCT Application Publication No. WO 02/92599

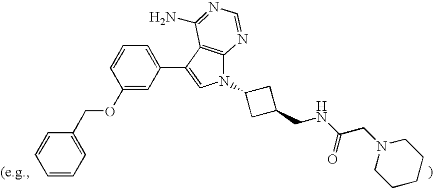

or any flavonoid glycone such as quercetin (see e.g., PCT Application Publication No. WO 03/39538) is provided and/or administered in association with the anti-IGF1R formulation of the invention.

In an embodiment of the invention, the anti-IGF1R formulation of the invention is provided and/or administered in association with

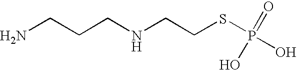

### (Amifostine);

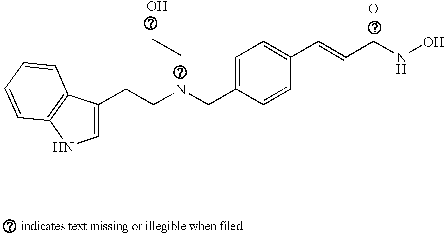

### (NVP-LAQ824; Atadja et al., Cancer Research 64: 689-695 (2004)),

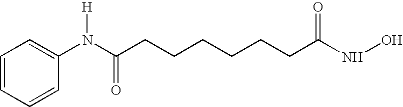

(suberoyl analide hydroxamic acid),

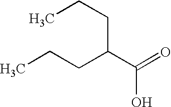

(Valproic acid; Michaelis et al., Mol. Pharmacol, 65:520-527 (2004)),

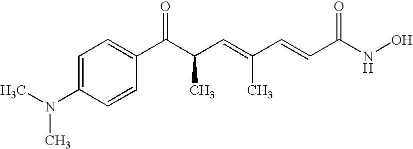

(trichostatin A),

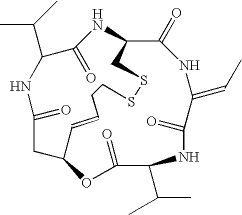

### (FK-228; Furumai et al., Cancer Research 62: 4916-4921 (2002)), or

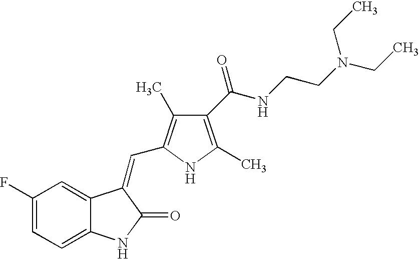

(SU11248; Mendel etal., Clin. Cancer Res. 9(1):327-37 (2003)).

In an embodiment of the invention, the anti-IGF1R formulation of the invention is provided and/or administered in association with a progestational agent such as

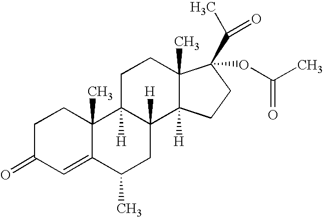

(medroxyprogesterone acetate; sold as Provera® by Pharmacia & Upjohn Co.; Kalamazoo, Mich.), or

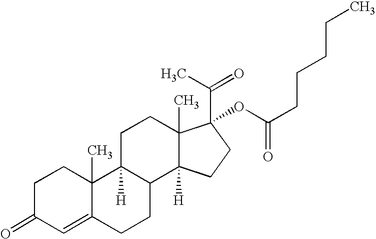

(hydroxyprogesterone caproate; 17-((1-Oxohexyl)oxy)pregn-4-ene-3,20-dione;).

In an embodiment of the invention, the anti-IGF1R formulation of the invention is provided and/or administered in association with

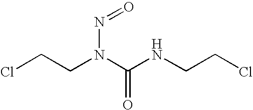

### (Carmustine); or

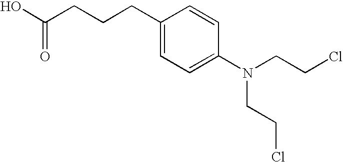

### (Chlorambucil).

Agents which inhibit IGF production, which, in an embodiment of the invention, are provided and/or administered in association with the anti-IGF1R formulation of the invention, include octreotide (L-Cysteinamide, D-phenylalanyl-L-cysteinyl-L-phenylalanylD-tryptophyl-L-lysyl-L-threonyl-N-[2-hydroxy-1-(hydroxymethyl) propyl]-, cyclic (2_{—}7)- disulfide; [R R*,R*)];

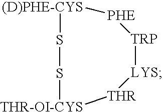

Katz et al., Clin Pharm. 8(4):255-73 (1989); sold as Sandostatin LAR® Depot; Novartis Pharm. Corp; E. Hanover, N.J.).

In an embodiment of the invention, a proteasome inhibitor, such as bortezomib

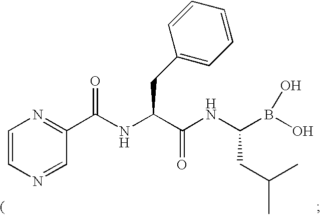

[(1R)-3-methyl-1-[[(2S)-1-oxo-3-phenyl-2-[(pyrazinylcarbonyl) amino]propyl]amino]butyl] boronic acid; sold as Velcade™; Millennium Pharm., Inc.; Cambridge, Mass.), is provided and/or administered in association with the anti-IGF1R formulation of the invention.

In an embodiment of the invention, a microtubule stabilizer or microtubule depolymerizer/inhibitor such as paclitaxel

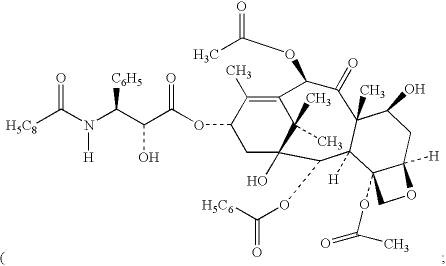

sold as Taxol®; Bristol-Myers Squibb; New York, N.Y.), docetaxel

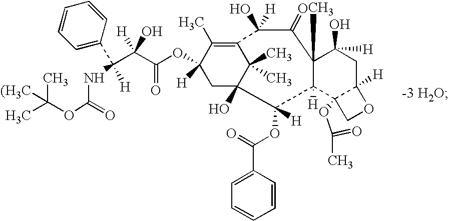

sold as Taxotere®; Aventis Pharm, Inc.; Bridgewater, N.J.); vincristine

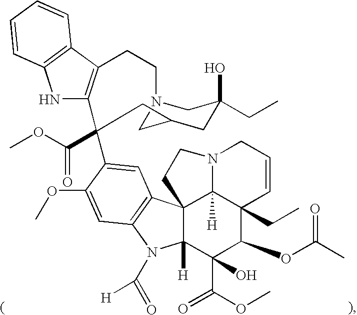

vinblastine

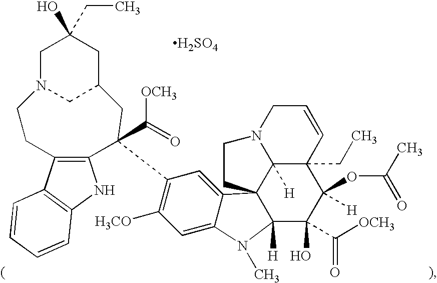

epothilone B and BMS-247550

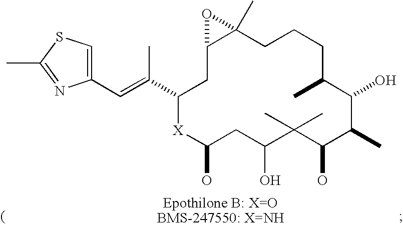

Lee et al., Clin. Cancer Res. 7(5):1429-37 (2001)), any podophyllotoxin or derivatives thereof including Etoposide (VP-16;

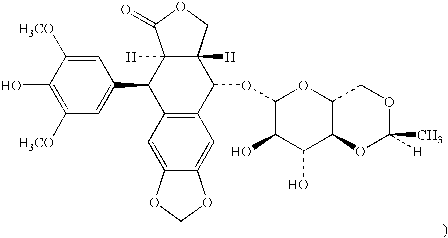

### or BMS-310705

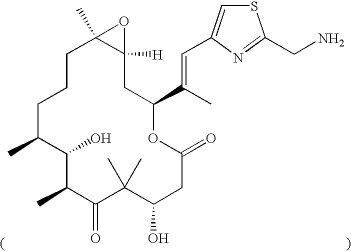

is provided and/or administered in association with the anti-IGF1R formulation of the invention.

In an embodiment of the invention, the anti-IGF1R formulation of the invention is provided in association with any of one or more compounds as set forth in U.S. Pat. No. 5,260,291. For example, in an embodiment of the invention, the compound is a [³H-imidazo-5,1-d]-1,2,3,5-tetrazin-4-one derivative represented by the structural formula:

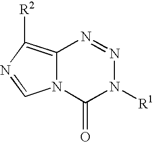

wherein R¹ represents a hydrogen atom, or a straight- or branched-chain alkyl (e.g., —CH₃), alkenyl or alkynyl group containing up to 6 carbon atoms, each such group being unsubstituted or substituted by from one to three substituents selected from halogen (i.e., bromine, iodine or, preferably, chlorine or fluorine) atoms, straight- or branched-chain alkoxy, (e.g., methoxy), alkylthio, alkylsullihinyl and alkylsulphonyl groups containing up to 4 carbon atoms, and optionally substituted phenyl groups, or R¹ represents a cycloalkyl group, and R² represents a carbamoyl group which may carry on the nitrogen atom one or two groups selected from straight- and branched-chain alkyl and alkenyl groups, each containing up to 4 carbon atoms, and cycloalkyl groups, e.g., a methylcarbamoyl or dimethylcarbamoyl group.

When the symbol R¹ represents an alkyl, alkenyl or alkynyl group substituted by two or three halogen atoms, the aforesaid halogen atoms may be the same or different. When the symbol R¹ represents an alkyl, alkenyl or alkynyl group substituted by one, two or three optionally substituted phenyl groups the optional substituents on the phenyl radical(s) may be selected from, for example, alkoxy and alkyl groups containing up to 4 carbon atoms (e.g., methoxy and/or methyl group(s)) and the nitro group; the symbol R¹ may represent, for example, a benzyl or p-methoxybenzyl group. Cycloalkyl groups within the definitions of symbols R¹ and R² contain 3 to 8, preferably 6, carbon atoms.

In an embodiment, tetrazine derivatives of the structural formula

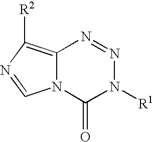

are those wherein R¹ represents a straight- or branched-chain alkyl group containing from 1 to 6 carbon atoms optionally substituted by one or two halogen (preferably chlorine, fluorine or bromine) atoms or by an alkoxy group containing 1 to 4 carbon atoms (preferably methoxy) or by a phenyl group (optionally substituted by one or two alkoxy groups containing from 1 to 4 carbon atoms, preferably methoxy), or R¹ represents an alkenyl group containing 2 to 6 carbon atoms (preferably allyl) or a cyclohexyl group.

In an embodiment, tetrazine derivatives are those of structural formula

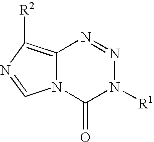

wherein R¹ represents a straight- or branched-chain alkyl group containing from 1 to 6 carbon atoms, and more especially from 1 to 3 carbon atoms, unsubstituted or substituted by a halogen, preferably chlorine or fluorine, atom. In an embodiment, R¹ represents a methyl or 2-haloalkyl, e.g., 2-fluoroethyl or, preferably, 2-chloroethyl, group.

In an embodiment, R² represents a carbamoyl group or a monoalkylcarbamoyl, e.g., methylcarbamoyl, or monoalkenylcarbamoyl group.

Temozolomide

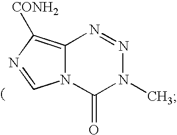

sold by Schering Corp.; Kenilworth, N.J. as Temodan®) 8-carbamoyl-3-methyl-[3H]-imidazo[5,1-d]-1,2,3,5-tetrazin-4-one; 8-carbamoyl-3-n-propyl-[3H]-imidazo[5,1-d]-1,2,3,5-tetrazin-4-one; 8-carbamoyl-3-(2-chloroethyl)-[3H]-imidazo-[5,1-d]-1,2,3,5-tetrazin-4-one; 3-(2-chloroethyl)-8-methylcarbamoyl-[3H]-imidazo[5,1-d]-1,2,3,5-tetrazin-4-one; 8-carbamoyl-3-(3-chloropropyl)-[3H]-imidazo-[5,1-d]-1,2,3,5-tetrazin-4-one; 8-carbamoyl-3-(2,3-dichloropropyl)-[3H]-imidazo[5,1-d]-1,2,3,5-tetrazin-4-one; 3-allyl-8-carbamoyl-[3H]-imidazo[5,1-d]-1,2,3,5-tetrazin-4-one; 3-(2-chloroethyl)-8-dimethylcarbamoyl-[3H]-imidazo[5,1-d]-1,2,3,5-tetrazin-4-one; 3-(2-bromoethyl)-8-carbamoyl-[3H]-imidazo-[5,1-d]-1,2,3,5-tetrazin-4-one; 3-benzyl-8-carbamoyl-[3H]-imidazo[5,1-d]-1,2,3,5-tetrazin-4-one; 8-carbamoyl-3-(2-methoxyethyl)-[3H]-imidazo[5,1-d]-1,2,3,5-tetrazin-4-one; 8-carbamoyl-3-cyclohexyl-[3H]-imidazo[5,1-d]-1,2,3,5-tetrazin-4-one; or 8-carbamoyl-3-(Wmethoxybenzyl)-[3H]imidazo[5,1-d]-1,2,3,5-tetrazin-4-one is, in an embodiment of the invention, administered and/or provided with the anti-IGF1R formulation of the invention.

Anthracyclines which, in an embodiment of the invention, are provided and/or administered in association with the anti-IGF1R formulation of the invention include doxorubicin

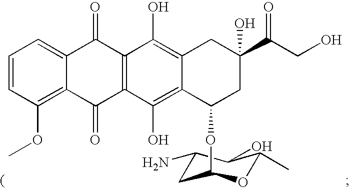

sold as Doxil®; Ortho Biotech Products L.P.; Raritan, N.J.); daunorubicin

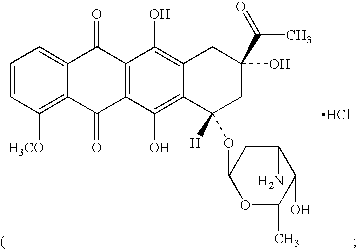

sold as Cerubidine®; Ben Venue Laboratories, Inc.; Bedford, Ohio) and epirubicin

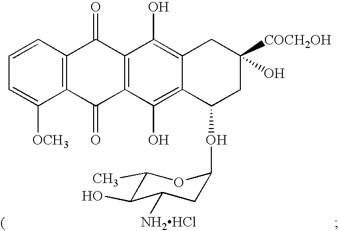

sold as Ellence®; Pharmacia & Upjohn Co; Kalamazoo, Mich.).

In an embodiment of the invention, the anti-IGF1R formulation of the invention is provided and/or administered in association with an anti-androgen including, but not limited to:

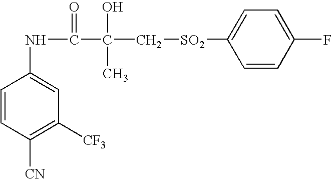

(bicalutamide; sold at CASODEX® by AstraZeneca Pharmaceuticals LP; Wilmington, Del.);

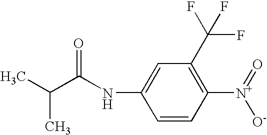

(flutamide; 2-methyl-N-[4-nitro-3 (trifluoromethyl) phenyl] propanamide; sold as Eulexin® by Schering Corporation; Kenilworth, N.J.);

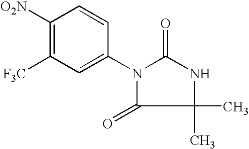

(nilutamide; sold as Nilandron® by Aventis Pharmaceuticals Inc.; Kansas City, Mo.) and

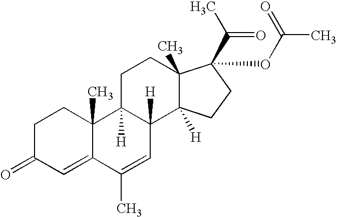

(Megestrol acetate; sold as Megace® by Bristol-Myers Squibb).

In an embodiment of the invention, the anti-IGF1R formulation of the invention is provided in association with

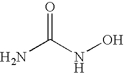

### (Hydroxyurea);

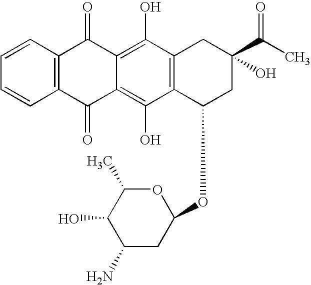

### (Idarubicin);

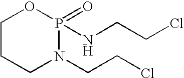

### (Ifosfamide);

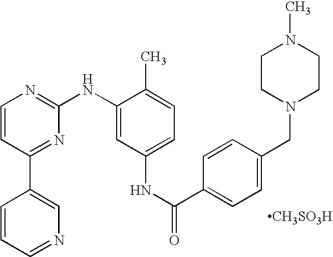

(Imatinib; sold as Gleeveo® by Novartis Pharmaceuticals Corporation; East Hanover, N.J.);

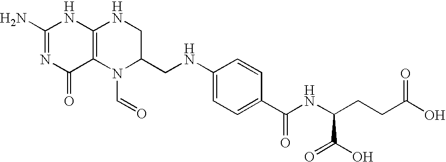

### (Laucovorin);

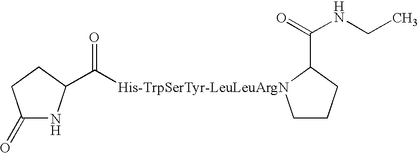

### (Leuprolide);

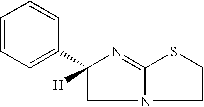

### (Levamisole);

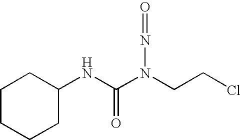

### (Lomustine);

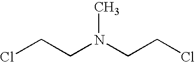

### (Mechlorethamine);

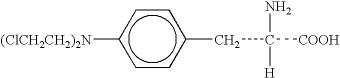

(Melphalan: sold as Alkeran® by Celegene Corporation; Warren, N.J.);

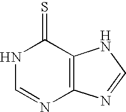

### (Mercaptopurina);

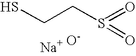

### (Mesna);

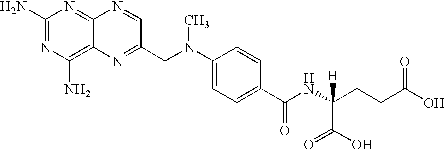

### (Methotrexate);

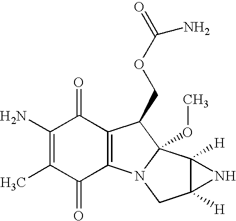

### (Mitomycin);

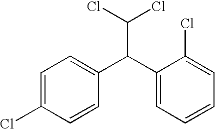

### Cl (Mitotane); or

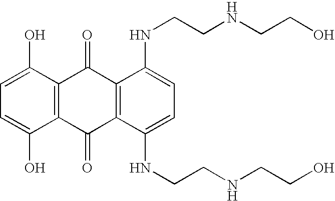

### (Mitoxantrone).

In an embodiment of the invention, the anti-IGF1R formulation of the invention is provided and/or administered in association with);;

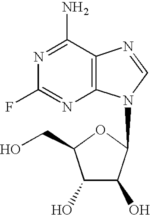

### (Fludarabine);

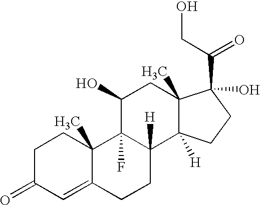

### (Fludrocortisone);

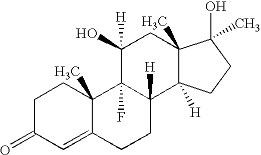

### (Fluoxymesterone).

In an embodiment, the anti-IGF1R formulation of the invention is provided and/or administered in association with

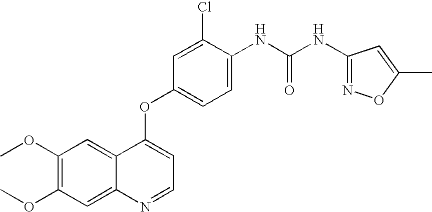

### (KRN951),

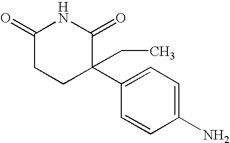

### (Aminoglutethimide);

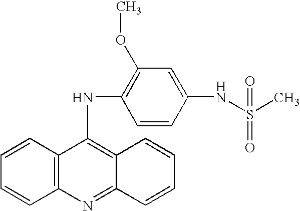

### (Amsacrine);

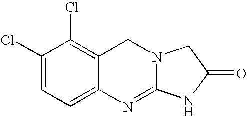

### (Anagrelide);

Anti-estrogens and selective estrogen receptor modulators (SERMs), which, in an embodiment of the invention, are administered and/or provided in association with an anti-IGF1R formulation of the invention include droloxifene (3-hydroxytamoxifen), 4-hydroxytamifen

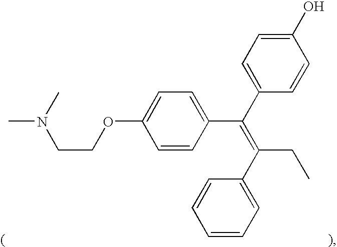

tamoxifen

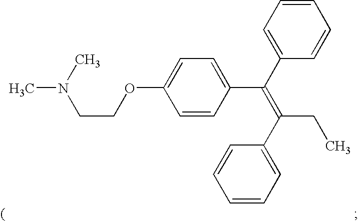

sold as Nolvadex®; Astra-Zenaca; Wilmington, Del.); pipendoxifene

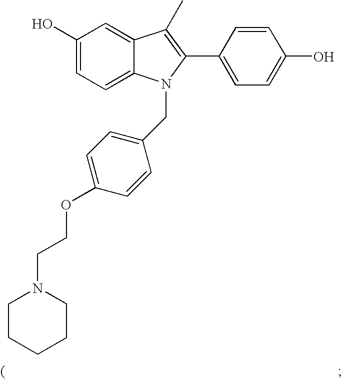

ERA-923; Greenberger et al., Clin. Cancer Res. 7(10):3166-77 (2001)); arzoxifene

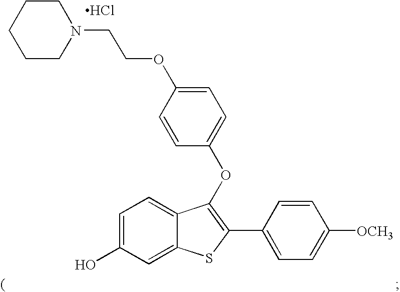

LY353381; Sato et al., J. Pharmacol. Exp. Ther. 287(1):1-7 (1998)); raloxifene

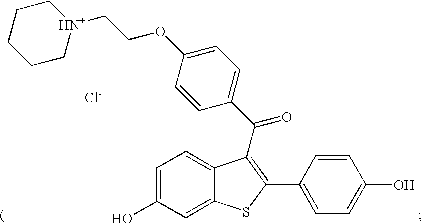

sold as Evista®; Eli Lilly & Co.; Indianapolis, Ind.); fulvestrant

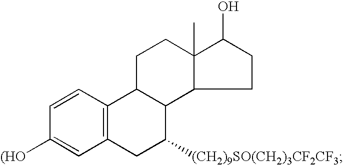

ICI-182780; sold as Faslodex; Astra Zeneca; Wilmington, Del.); acolbifene (EM-652;

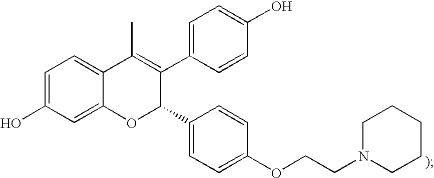

toremifine

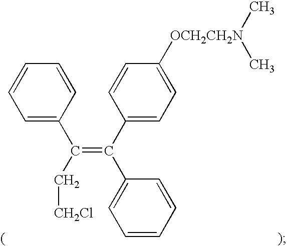

lasofoxifene (CP-336, 156;

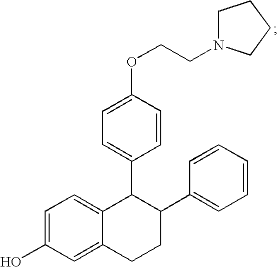

Ke et al., Endocrinology 139(4):2068-76 (1998)); idoxifene (pyrrolidino-4-iodotamoxifen;

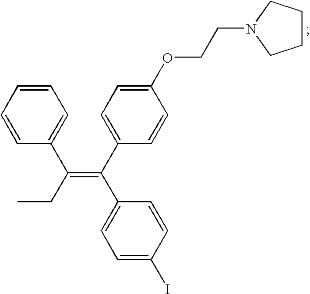

### Nuttall et al., Endocrinology 139(12):5224-34 (1998)); TSE-424

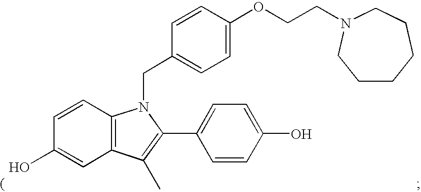

Aromatase inhibitors, which can be included with the anti-IGF1R formulation of the invention, include anastrazole

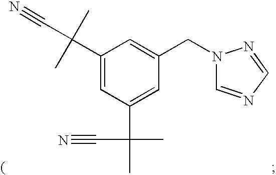

Dukes et al., J. Steroid. Biochem. Mol. Biol. 58(4):439-45 (1996)), letrozole

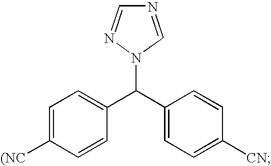

sold as Femara®; Novartis Pharmaceuticals Corp.; E. Hanover, N.J.) and exemestane

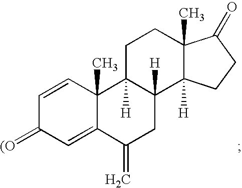

sold as Aromasiri®; Pharmacia Corp.; Kalamazoo, Mich.).

The anti-IGF1R formulation of the invention is, in an embodiment of the invention, provided and/or administered in association with gemcitabine HCl

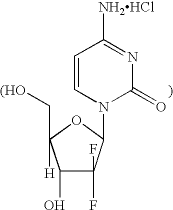

with 13-cis-retinoic acid

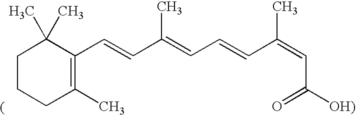

or with any IGFR inhibitor set forth in any of Mitsiades et al., Cancer Cell 5:221-230 (2004); Garcia-Echeverria et. al., Cancer Cell 5:231-239,2004; WO 2004/030627 or WO 2004/030625.

In an embodiment of the invention, the anti-IGF1R formulation of the invention is provided and/or administered in association with

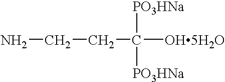

(Pamidronate; sold as Aredia® by Novartis Pharmaceuticals Corporation; East Hanover, N.J.);

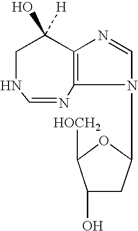

(Pentostatin; sold as Nipent® by Supergen; Dublin, Calif.);

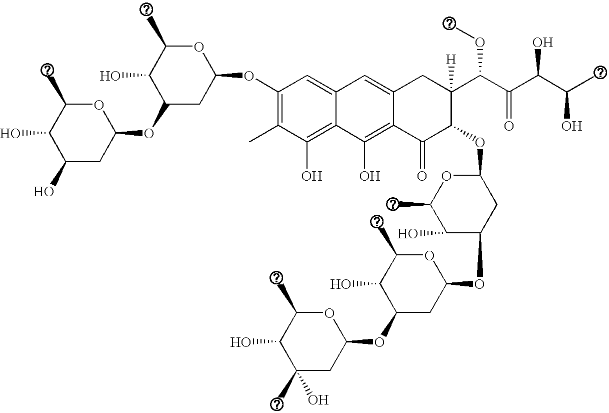

### (Plicamycin);

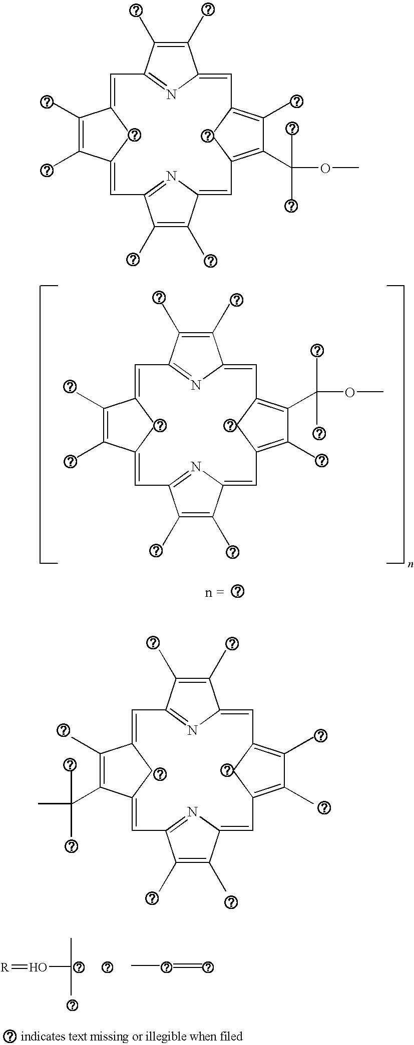

(Porfimer; sold as Photofrin® by Axcan Scandipham Inc.; Birmingham, Ala.);

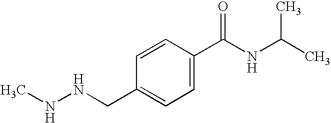

### (Procarbazine);

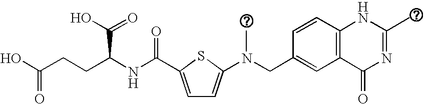

(Raltitrexed); Rituximab (sold as Rituxan® by Genetech, Inc.; South San Francisco, Calif.

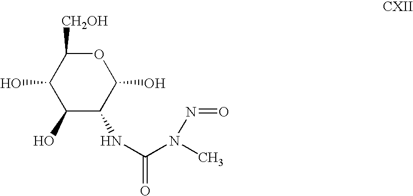

### (Streptozocin);

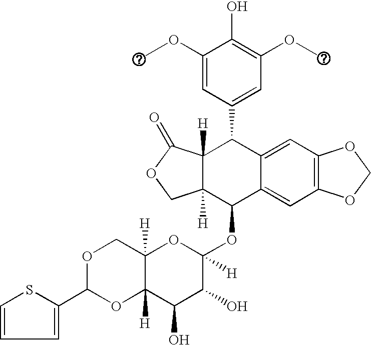

### (Teniposide);

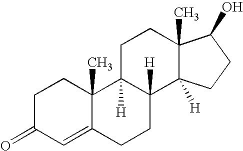

### (Testosterone);

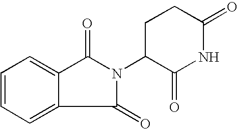

### (Thalidomide);

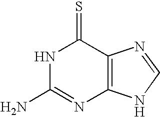

### (Thioguanine);

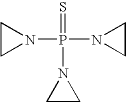

### (Thiotepa);

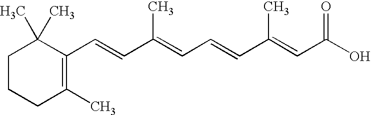

### (Tretinoin); or

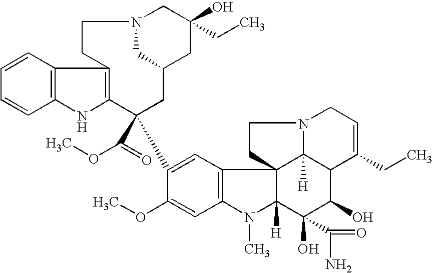

### (Vindesine).

In an embodiment of the invention, the anti-IGF1R formulation of the invention is provided and/or administered in association with one or more of any of: pegylated or unpegylated interferon alfa-2a, pegylated or unpegylated interferon alfa-2b, pegylated or unpegylated interferon alfa-2c, pegylated or unpegylated interferon alfa n-1, pegylated or unpegylated interferon alfa n-3 and pegylated, unpegylated consensus interferon or albumin-interferon-alpha.

Topoisomerase inhibitors which, in an embodiment of the invention, are provided and/or administered in association with an anti-IGF1R formulation of the invention include camptothecin

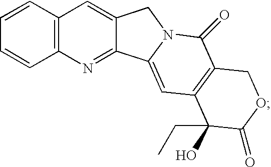

Stork et al., J. Am. Chem. Soc. 93(16): 4074-4075 (1971); Beisler et al., J. Med. Chem. 14(11): 1116-1117 (1962)), topotecan

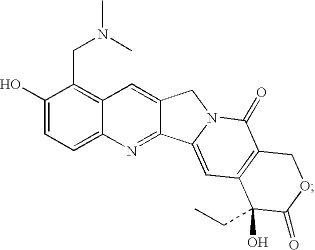

sold as Hycamtin®; GlaxoSmithKline, Research Triangle Park, N.C.; Rowinski et al., J. Clin. Oncol. 10(4): 647-656 (1992)), etoposide

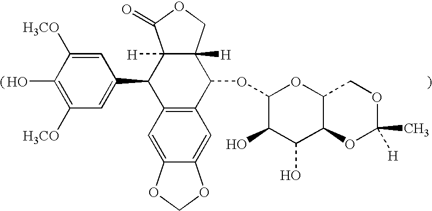

and irinotecan

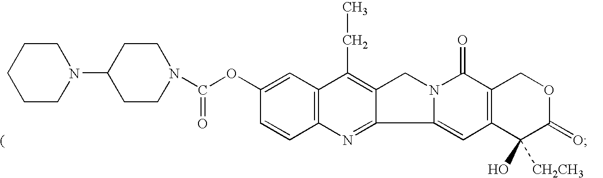

sold as Campotosar®; Pharmacia & Upjohn Co.; Kalamazoo. Mich.).

In an embodiment, an IGF1R1 inhibitory agent provided and/or administered in association with the anti-IGF1R formulation of the invention includes AEW-541 (NVP-AEW-541; NVP-AEW-541-NX-7):

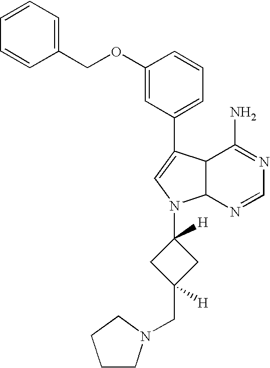

(novartis, East Hanover, N.J.; see WO 2002/92599); or

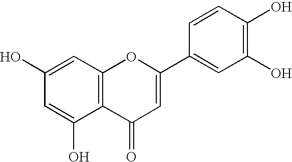

### WO 2002/39538).

In an embodiment of the invention the anti-IGF1R formulation of the invention is provided and/or administered in association with any kinase inhibitor compound set forth in published international applications WO 2004/030627 or WO 2004/030625. In an embodiment, the kinase inhibitor is (±)-4-[2-(3-chloro-4-fluoro-phenyl)-2-hydroxy-ethylamino]-3-[6-(imidazol-1-yl)-4-methyl-1H-benzimidazol-2-yl]-1H-pyridin-2-one:

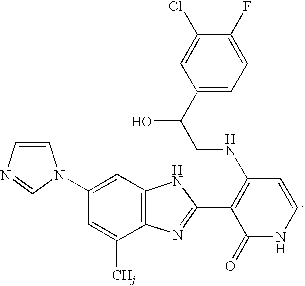

Antisense oligonucleotides can be produced that are complementary to the mRNA of the IGF1R, IGF-1 or IGF-2 gene and can be used to inhibit transcription or translation of the genes. Production of antisense oligonucleotides effective for therapeutic uses is well known in the art. Antisense oligonucleotides are often produced using derivatized or modified nucleotides in order to increase half-life or bioavailability. The primary sequence of the IGF1R, IGF-1 or IGF-2 gene can also be used to design ribozymes. Most synthetic ribozymes are generally hammerhead, tetrahymena and haripin ribozymes. Methods of designing and using ribozymes to cleave specific RNA species are well known in the art. In an embodiment of the invention, the anti-IGF1R formulation of the invention is provided and/or administered in association with the anti-sense IGF1R nucleic acid ATL-1101 (Antisense Therapeutics Ltd; Australia). In an embodiment, the IGF1R anti-sense nucleic acid comprise any of the following nucleotide sequences: 5′-ATCTCTCCGCTTCCTTTC-3′ (SEQ ID NO: 18), 5′-ATCTCTCCGCTTCCTTTC-3′ (SEQ ID NO: 19), 5′-ATCTCTCCGCTTCCTTTC-3′ (SEQ ID NO: 20) or any IGFR antisense nucleic acid set forth in any of US Published Patent Application No. US20030096769; Published International Application No. WO 2003/100059 Fogarty et al., Antisense Nucleic Acid Drug Dev. 2002 December; 12(6):369-77; White et al., J Invest Dermatol. 2002 June; 118(6):1003-7; White et al., Antisense Nucleic Acid Drug Dev. 2000 June; 10(3):195-203; or Wraight et al., Nat Biotechnol. 2000 May; 18(5):521-6.

The chemical structures and other useful information regarding many of the foregoing agents can be found in the Physicians' Desk Reference, 57^{th} ed., 2003; Thompson P D R; Montvale, N.J.

Categorization of a particular agent into a particular class (e.g., FPT inhibitor or microtubule stabilizer) is only done for descriptive purposes and is not meant to limit the invention in any way.

The scope of the present invention also includes administration of compositions comprising the anti-IGF1R formulation of the invention in association with one or more other chemotherapeutic agents (e.g., as described herein) and in association with one or more antiemetics including, but not limited to, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co.; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by Glaxosmithkline; Research Triangle Park, N.C.), dolasetron (sold as Anzemet® by Sanofi-Aventis; New York, N.Y.), or tropisetron (sold as Navoban® by Novartis; East Hanover, N.J.).

The scope of present invention includes treatment methods comprising administration of compositions comprising the anti-IGF1R formulation of the invention along with one or more of the foregoing chemotherapeutic agents or any salt, hydrate, isomer, formulation, solvate or prodrug thereof.

The scope of the present invention also includes administration of the anti-IGF1R formulation of the invention in association with any anti-cancer procedure including, but not limited to, surgical tumorectomy or anti-cancer radiation therapy.

### Dosage and Administration

Methods of the present invention include provision and/or administration of an IGF1R antibody in a pharmaceutical formulation as set forth herein, optionally in association with a further therapeutic agent, or a pharmaceutical composition thereof to treat or prevent cancer or any medical disorder mediated by IGF1R, IGF-1 and/or IGF-2. Typically, the administration and dosage of such further agents is, when possible, done according to the schedule listed in the product information sheet of the approved agents, in the Physicians' Desk Reference 2003 (Physicians' Desk Reference, 57th Ed); Medical Economics Company; ISBN: 1563634457; 57th edition (November 2002), as well as therapeutic protocols well known in the art.

In an embodiment, a formulation of the invention is administered to a subject parenterally, for example, by intravenous, intrathecal, subcutaneous, intramuscular, intratumoral or intraarterial injection. In an embodiment, the formulation is administered orally or by inhalation. In an embodiment of the invention, a formulation of the invention comprising a single-chain anti-IGF1R antibody of the invention is administered pulmonarily by inhalation.

The term “cancer” includes, but is not limited to, neuroblastoma, rhabdomyosarcoma, osteosarcoma, any pediatric cancer, acromegaly, ovarian cancer, pancreatic cancer, benign prostatic hyperplasia, breast cancer, prostate cancer, bone cancer, lung cancer, gastric cancer, colorectal cancer, cervical cancer, synovial sarcoma, bladder cancer, Wilm's cancer, ovarian cancer, benign prostatic hyperplasia (BPH), diarrhea associated with metastatic carcinoid and vasoactive intestinal peptide secreting tumors (e.g., VIPoma or Werner-Morrison syndrome), kidney cancer (e.g., renal cell carcinoma or transitional cell cancer), Ewing Sarcoma, leukemia (e.g., acute lymphoblastic leukemia (e.g., B-precursor type or T-cell type)) or brain cancer (e.g., glioblastoma or a non-glioblastoma) including meningiomas, pituitary adenomas, vestibular schwannomas, primitive neuroectodermal tumors, medulloblastomas, astrocytomas, oligodendrogliomas, ependymomas, and choroid plexus papillomas and any metastatic tumor thereof. Acromegaly may also be treated with a composition of the invention. Antagonism of IGF-I has been reported for treatment of acromegaly (Drake, et al., (2001) Trends Endocrin. Metab. 12: 408-413). Other non-malignant medical conditions which may also be treated, in a subject, by administering a formulation of the invention, include gigantism, psoriasis, atherosclerosis, smooth muscle restenosis of blood vessels or inappropriate microvascular proliferation, such as that found as a complication of diabetes, especially of the eye rheumatoid arthritis, Grave's disease, multiple sclerosis, systemic lupus erythematosus, Hashimoto's thyroiditis, myasthenia gravis, auto-immune thyroiditis and Bechet's disease.

The term “therapeutically effective amount” or “therapeutically effective dosage” means that amount or dosage of a composition of the invention (e.g., anti-IGF1R antibody in a formulation of the invention) that will elicit a biological or medical response of a tissue, system, subject or host that is being sought by the administrator (such as a researcher, doctor or veterinarian) which includes any measurable alleviation of the signs, symptoms and/or clinical indicia of a medical disorder, such as cancer (e.g., tumor growth and/or metastasis) including the prevention, slowing or halting of progression of the medical disorder to any degree. For example, in one embodiment, a therapeutically effective amount is an amount that is sufficient to yield a therapeutic serum concentration. A “therapeutic serum concentration” is defined in this context as a concentration sufficient to achieve 50% tumor growth inhibition; preferably 60% tumor growth inhibition; more preferably 65% tumor growth inhibition (Plowman et al., Anticancer Drug Development Guide: Preclinical Screening, Clinical Trails, and Approval, Edited by: B. Teicher; Humana Press Inc., Totowa, N.J.).

In one embodiment, a “therapeutically effective dosage” or “therapeutically effective amount” of any anti-IGF1R antibody (e.g., an anti-IGF1R antibody comprising mature LCC, LCD, LCE or LCF light chain and/or mature HCA or HCB heavy chain) is an amount sufficient to yield a therapeutic serum concentration of at least about 19 μg/mL in the subject being treated throughout the treatment period. In one embodiment, the amount is administered at a time interval selected from the group consisting of once per week, twice per week, once every two weeks and once every three weeks. Doses of at least about 3 mg/kg of body weight per week are preferred for most cases. In other embodiments, doses of at least about 4 mg/kg, 5 mg/kg or 6 mg/kg are preferred. Doses between about 3 mg/kg of body weight and about 100 mg/kg of body weight per week are more preferred. Doses between about 3 mg/kg of body weight and about 30 mg/kg of body weight are further preferred.

In another embodiment, a “therapeutically effective dosage” of any anti-IGF1R antibody (e.g., an anti-IGF1R antibody comprising mature LCC, LCD, LCE or LCF light chain and/or mature HCA or HCB heavy chain) is between about 0.3-20 mg/kg of body weight (e.g., about 0.3 mg/kg of body weight, about 0.6 mg/kg of body weight, about 0.9 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, about 20 mg/kg of body weight), about once per week to about once every 3 weeks (e.g., about once every 1 week or once every 2 weeks or once every 3 weeks). As mentioned above, the therapeutically effective dosage of a further therapeutic agent is, when possible, as set forth in the Physicians' Desk Reference.

Dosage regimens may be adjusted to provide the optimum desired response (e.g., a therapeutic response). For example, a single dose may be administered or several divided doses may be administered over time or the dose may be proportionally reduced or increased as indicated by exigencies of the therapeutic situation. For example, dosage may be determined or adjusted, by a practitioner of ordinary skill in the art (e.g., physician or veterinarian) according to the patient's age, weight, height, past medical history, present medications and the potential for cross-reaction, allergies, sensitivities and adverse side-effects. It is especially advantageous to formulate parenteral compositions in dosage unit forms for ease of administration and uniformity of dosage.

For example, a unit dosage form includes a pharmaceutical composition comprising a complete dose or group of doses of an antibody or antigen-binding fragment thereof and pharmaceutically acceptable carrier. For example, the term includes syringe unit dosage forms including a syringe loaded with a single full dose of the antibody or fragment. The term also includes a vial including one full dose or several compete doses (e.g., 1, 2, 3, 4, 5, 6, 7, 8, 9, 10). For example, in an embodiment of the invention, if the intended dose of an antibody or fragment is 10 mg/kg of body weight and the average body weight of a patient is 65 kg, then a unit dosage form may include 650 kg of the antibody or fragment. Unit dosage forms may be fabricated for individual patients, for example, if the patient weighs 85 kg, then a single unit dosage form would include 850 kg of antibody or fragment.

A physician or veterinarian having ordinary skill in the art can readily determine and prescribe the effective amount of the pharmaceutical composition required. For example, the physician or veterinarian could start doses of the antibody or antigen-binding fragment of the invention employed in the pharmaceutical composition at levels lower than that required in order to achieve the desired therapeutic effect and gradually increase the dosage until the desired effect is achieved. The effectiveness of a given dose or treatment regimen of an antibody or combination of the invention can be determined, for example, by determining whether a tumor being treated in the subject shrinks or ceases to grow. The size and progress of a tumor can be easily determined, for example, by X-ray, magnetic resonance imaging (MRI) or visually in a surgical procedure. In general, tumor size and proliferation can be measured by use of a thymidine PET scan (see e.g., Wells et al., Clin. Oncol. 8: 7-14 (1996)). Generally, the thymidine PET scan includes the injection of a radioactive tracer, such as [2-¹¹C]-thymidine, followed by a PET scan of the patient's body (Vander Borght et al., Gastroenterology 101: 794-799, 1991; Vander Borght et al., J. Radiat. Appl. Instrum. Part A, 42: 103-104 (1991)). Other tracers that can be used include [¹⁸F]-FDG (18-fluorodeoxyglucose), [¹²⁴I]IUdR (5-[124I]iodo-2′-deoxyuridine), [⁷⁶Br]BrdUrd (Bromodeoxyuridine), [¹⁸F]FLT (3′-deoxy-3′fluorothymidine) or [¹¹C]FMAU (2′-fluoro-5-methyl-1-β-D-arabinofuranosyluracil).

For example, neuroblastoma progress can be monitored, by a physician or veterinarian, by a variety of methods, and the dosing regimen can be altered accordingly. Methods by which to monitor neuroblastoma include, for example, CT scan (e.g., to monitor tumor size), MRI scan (e.g., to monitor tumor size), chest X-ray (e.g., to monitor tumor size), bone scan, bone marrow biopsy (e.g., to check for metastasis to the bone marrow), hormone tests (levels of hormones like epinephrine), complete blood test (CBC) (e.g., to test for anemia or other abnormality), testing for catecholamines (a neuroblastoma tumor marker) in the urine or blood, a 24 hour urine test for check for homovanillic acid (HMA) or vanillyl mandelic acid (VMA) levels (neuroblastoma markers) and an MIBG scan (scan for injected I¹²³-labeled metaiodobetaguanidine; e.g., to monitor adrenal tumors).

For example, rhabdomyosarcoma progress can be monitored, by the physician or veterinarian, by a variety of methods, and the dosing regimen can be altered accordingly. Methods by which to monitor rhabdomyosarcoma include, for example tumor biopsy, CT scan (e.g., to monitor tumor size), MRI scan (e.g., to monitor tumor size), CT scan of the chest (e.g., to monitor metastases), bone scan (e.g., to monitor metastases), bone marrow biopsy (e.g., to monitor metastases), spinal tap (e.g., to check for metastasis into the brain) and a thorough physical exam.

For example, osteosarcoma progress can be monitored, by the physician or veterinarian, by a variety of methods, and the dosing regimen can be altered accordingly. Methods by which to monitor osteosarcoma include, for example, X-ray of the affected area or of the chest (e.g., to check for spread to the lungs), CT scan of the affected area, blood tests (e.g., to measure alkaline phosphatase levels), CT scan of the chest to see if the cancer has spread to the lungs, open biopsy, or a bone scan to see if the cancer has spread to other bones.

For example, pancreatic cancer progress can be monitored, by the physician or veterinarian, by a variety of methods, and the dosing regimen can be altered accordingly. Methods by which to monitor pancreatic cancer include blood tests to check for tumor markers CA 19-9 and/or carcinoembryonic antigen (CEA), an upper GI series (e.g., a barium swallow), endoscopic ultrasonography; endoscopic retrograde cholangiopancreatography (an x-ray of the pancreatic duct and bile ducts); percutaneous transhepatic cholangiography (an x-ray of the bile duct), abdominal ultrasound imaging or abdominal CT scan.

For example, bladder cancer progress can be monitored, by the physician or veterinarian, by a variety of methods, and the dosing regimen can be altered accordingly. Methods by which to monitor bladder cancer include urinalysis to detect elevated levels of tumor markers (e.g., nuclear matrix protein (NMP22)) in the urine, urinalysis to detect microscopic hematuria, urine cytology to detect cancer cells by examining cells flushed from the bladder during urination, bladder cystoscopy, intravenous pyelogram (IVP), retrograde pyelography, chest X ray to detect metastasis, computed tomography (CT), bone scan, MRI scan, PET scan or biopsy.

For example, breast cancer progress can be monitored, by the physician or veterinarian, by a variety of methods, and the dosing regimen can be altered accordingly. Methods by which to monitor breast cancer include mammography, aspiration or needle biopsy or palpation.

For example, lung cancer progress can be monitored, by the physician or veterinarian, by a variety of methods, and the dosing regimen can be altered accordingly. Methods by which to monitor lung cancer include chest X-ray, CT scan, low-dose helical CT scan (or spiral CT scan), MRI scan, PET scan, bone scan, sputum cytology, bronchoscopy, mediastinoscopy, biopsy (e.g., needle or surgical), thoracentesis or blood tests to detect PTH (parathyroid hormone), CEA (carcinogenic antigen) or CYFRA21-1 (cytokeratin fragment 19).

For example, prostate cancer progress can be monitored, by the physician or veterinarian, by a variety of methods, and the dosing regimen can be altered accordingly. Methods by which to monitor prostate cancer include digital rectal examination, transrectal ultrasound, blood tests taken to check the levels of prostate specific antigen (PSA) and prostatic acid phosphatase (PAP), biopsy, bone scan and CT scan.

For example, colorectal or colon cancer progress can be monitored, by the physician or veterinarian, by a variety of methods, and the dosing regimen can be altered accordingly. Methods by which to monitor colorectal or colon cancer include CT scan, MRI scan, chest X-ray, PET scan, fecal occult blood tests (FOBTs), flexible proctosigmoidoscopy, total colonoscopy, and barium enema.

For example, cervical cancer progress can be monitored, by the physician or veterinarian, by a variety of methods, and the dosing regimen can be altered accordingly. Methods by which to monitor cervical cancer include PAP smear, pelvic exam, colposcopy, cone biopsy, endocervical curettage, X-ray, CT scan, cystoscopy and proctoscopy.

For example, gastric cancer progress can be monitored, by the physician or veterinarian, by a variety of methods, and the dosing regimen can be altered accordingly. Methods by which to monitor gastric cancer include esophagogastroduodenoscopy (EGD), double-contrast barium swallow, endoscopic biopsy, computed tomographic (CT) scanning, magnetic resonance imagine (MRI) or endoscopic ultrasonography (EUS).

For example, Wilm's cancer progress can be monitored, by the physician or veterinarian, by a variety of methods, and the dosing regimen can be altered accordingly. Methods by which to monitor Wilm's cancer include abdominal computer tomography scan (CT), abdominal ultrasound, blood and urine tests to evaluate kidney and liver function, chest X-ray to check for metastasis, magnetic resonance imaging (MRI), blood tests and urinalysis to assay kidney function and biopsy.

In an embodiment of the invention, any patient suffering from a cancer whose tumor cells expresses IGF1R is selected for treatment with a formulation of the invention. In an embodiment of the invention, a patient whose tumor exhibits any of the following characteristics is selected for treatment with a formulation of the invention: IRS-1 phosphorylation on tyrosine 896; (ii) IRS-1 phosphorylation on tyrosine 612; (iii) IRS-1 phosphorylation on any tyrosine; (iv) IGF-II; and/or (v) IGF1R phosphorylation on any tyrosine. Such characteristics can be identified in an tumor cell by any of several methods commonly known in the art (e.g., ELISA or western blot).

### Kits

The kits of the present invention also include an anti-IGF1R antibody formulation of the invention along with information, for example in the form of a package insert, including information concerning the pharmaceutical compositions and dosage forms in the kit. Generally, such information aids patients and physicians in using the enclosed pharmaceutical compositions and dosage forms effectively and safely. For example, the following information regarding formulation can be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information. In an embodiment of the invention, wherein the formulation is provided in dry/lyophilized form, the kit includes sterile water or saline for reconstitution of the formulation into liquid form.

In a kit embodiment of the invention, the anti-IGF1R antibody of the invention is supplied in a vessel (e.g., a vessel that is internally sterile). In an embodiment of the invention, the formulation is in liquid form and in another embodiment of the invention, the formulation of in dry/lyophilized form. The vessel can take any form including, but not limited to, a glass (e.g., sintered glass) or plastic vial or ampule. For example, in an embodiment of the invention the glass is clear and in another embodiment of the invention, the glass is colored (e.g., amber) to block light from contacting the formulation. In an embodiment, the formulation is sparged with nitrogen or an inert gas (e.g., argon). The formulation, in an embodiment, is packaged in a sealed, air-tight vessel under an atmosphere of nitrogen or some inert gas. In an embodiment, the formulation is packaged in an air-tight vessel under vacuum. In an embodiment, the vessel containing the formulation comprises a resealable stopper (e.g., rubber) into which a needle may be inserted for removal of the formulation.

In an embodiment of the invention, the formulation of the invention is provided with an injectable device, for example, a syringe/hypodermic needle. In an embodiment, the syringe is pre-filled with the formulation of the invention (e.g., in liquid or dry/lyophilized form).

In an embodiment, the formulation of the invention is present in a vessel intended for intravenous infusion into the body of a subject. For example, in an embodiment of the invention, the vessel is a plastic infusion bag (e.g., polyvinylchloride or polyethylene).

### Examples

The following information is provided to more clearly describe the present invention and should not be construed to limit the present invention. Any and all of the compositions and methods described below fall within the scope of the present invention.

An exemplary formulation of anti-IGF-1R antibody for use in the present methods, hereinafter referred to as “LCF/HCA”, is detailed below in Examples 1 and 2. Example 3 describes two xenograph studies wherein LCF/HCA was administered at various doses to mouse tumor models in an assessment of the efficacy and pharmacokinetics of IP dosage of LCF/HCA in mice. The data from these studies are reported in Table 5. The xenograph studies show that the formulations described herein are effective at a minimum 130 nM serum concentration in mouse tumor models. Examples 4 and 5 describe studies evaluating the pharmacokinetics of LCF/HCA in cynomolgus monkeys. These studies establish that the 130 nM concentration is attainable at dosages of at least about 3 mg/kg of body weight, and that there is no substantial toxicity at dosages of up to 100 mg/kg of body weight in monkeys. The results of the monkey studies are summarized in Tables 6-19.

It is well known in the art that values of ±20% are acceptable in pharmacokinetic assessments for accuracy (% relative error [RE], mean bias) and interbatch precision (% coefficient of variation [CV]) (See, e.g., DeSilva et al., Pharmaceutical Research 20(11):1885-1900 (2003)).

### Example 1

### Formulation and Analysis of Anti-IGF1R Antibody

In this example, an antibody comprising mature light chain LCF (SEQ ID NO: 14 amino acids 20-128), mature heavy chain HCA (SEQ ID NO: 16 amino acids 20-137) and the constant regions (heavy chain γ1, light chain κ) (hereinafter “LCF/HCA”) was formulated as described and determined to exhibit superior stability characteristics (e.g., exhibiting stability at room temperature for several months).

### Method of Manufacture

### Materials

1. Sodium Acetate Trihydrate USP: 2.30 g per 1 L batch
2. Glacial Acetic Acid USP/Ph. Eur: 0.18 g per 1 L batch
3. Sucrose Extra Pure NF, Ph. Eur, BP: 70.0 g per 1 L batch
4. Antibody: 20.0 g per 1 L batch
5. Water for injection USP/Ph. Eur: quantity sufficient for 1 L volume
Note: the anti-IGF1R antibody may be susceptible to aggregation due to foaming and shaking. Avoid excess foaming during manufacturing, filtration and filling.

### Methods

### Compounding

Charged and dissolved sodium acetate trihydrate, acetic acid and sucrose in approximately 70% of batch volume of water for injection at room temperature in a stainless steel tank equipped with an agitator. To this solution, charged the required amount of drug substance (antibody) to the stainless steel vessel and agitated for at least 20 minutes. After agitating for 20 minutes, brough the batch to final volume with water for injection and allowed to agitate for another 20 minutes. Checked the pH of the solution. Aseptically filtered the solution through a sterilized filter (0.22 μm) into a sterilized stainless steel container. Aseptically filled into vials that had been washed and sterilized. Stoppered and crimped the vials with aluminum seals.

### Stability Testing

Two batches were manufactured according to the process described in the Compounding section.

The sealed vials from a prototype batch (Batch A) were placed on stability stations at the following conditions: 4 (4±2° C.; 60%±5% RH), 25H (25±2° C.; 60%±5% RH) and 40 (40±2° C., ambient RH) for 3 months. Initial samples and samples pulled at the end of each time-point were stored at 4° C. prior to analyses.

The sealed vials from a second batch (Batch B) were placed on the same stability stations as Batch A, in both the upright and inverted positions, for 6 months. Initial samples and samples pulled at the end of each time-point were stored at 4° C. prior to analyses.

TABLE 1A
Summary of assay results for anti-IGF1R antibody stability, Batch A.
Prototype 20 mM Ace pH 5.5 + 7% Sucrose

| Sample ID | Initial | 2 wk 40 C. | 4 wk 4 C. | 4 wk 25 C. | 4 wk 40 C. | 12 wk 4 C. | 12 wk 25 C. |
|---|---|---|---|---|---|---|---|
| PhysObs | clear | clear solution | clear solution | clear | clear | opalescent | Opalescent |
| | solution | contains | contains | solution | solution | solution | solution |
| | contains | particles | particles | contains | contains | contains | contains |
| | particles | | | particles | particles | particles | particles |
| pH | 5.4 | 5.4 | 5.4 | 5.3 | 5.4 | 5.4 | 5.4 |
| UV (mg/mL) | 22.34 | 22.75 | 24.78 | 23.40 | 22.43 | 22.49 | 23.06 |
| HPSEC | | | | | | | |
| % Monomer | 99.394 | 98.931 | 99.416 | 99.261 | 98.477 | 99.421 | 99.035 |
| % Early Eluting | 0.205 | 0.249 | 0.181 | 0.221 | 0.313 | 0.135 | 0.174 |
| % Late Eluting | 0.402 | 0.82 | 0.404 | 0.518 | 1.211 | 0.445 | 0.792 |
| SDS-PAGE | | | | | | | |
| Reducing | Heavy and | Heavy and | Heavy and | Heavy and | Heavy and | Heavy and | Heavy and |
| | light | light chains | light chains | light | light chains | light | light chains |
| | chains | detected | detected | chains | detected | chains | detected |
| | detected | under | under | detected | under | detected | under |
| | under | reducing | reducing | under | reducing | under | reducing |
| | reducing | conditions | conditions | reducing | conditions | reducing | conditions |
| | conditions | | | conditions | | conditions | |
| Non Reducing | Band | Band pattern | Band pattern | Band | Band | Band | Band |
| | pattern | matches | matches | pattern | pattern | pattern | pattern |
| | matches | typical non- | typical non- | matches | matches | matches | matches |
| | typical | reducing | reducing | typical | typical non- | typical | typical non- |
| | non- | antibody | antibody | non- | reducing | non- | reducing |
| | reducing | profile | profile | reducing | antibody | reducing | antibody |
| | antibody | | | antibody | profile | antibody | profile |
| | profile | | | profile | | profile | |
| Bio Assay | 21.4 | 18.3 | 14.0 | 17.2 | 11.8 | 23.3 | 29.2 |
| (mg/mL) | | | | | | | |
| HIAC | | | | | | | |
| Particle Size (≧10 μm) | 387 | | | | | 323 | 437 |
| (Particle | | | | | | | |
| count/container) | | | | | | | |
| Particle Size (≧25 μm) | 27 | | | | | 30 | 35 |
| (Particle | | | | | | | |
| count/container) | | | | | | | |
| Nanosizer | | | | | | | |
| Particle Size | 12.22 | 14.92 | 14.92 | 14.92 | 12.22 | 11.05 | 11.05 |
| (nm) | | | | | | | |

TABLE 1B
Summary of assay results for anti-IGF1R antibody stability, Batch B.

| | Sample ID | | | |
|---|---|---|---|---|
| | | 1 Month 5 C. | 1 Month 25 C. | 1 Month 40 C. |
| | Initial | (Upright) | (Upright) | (Upright) |
| Description | clear | opalescent | opalescent | opalescent |
| | solution | solution | solution | solution |
| | contains | contains | contains | contains |
| | particles | particles | particles | particles |
| pH | 5.5 | 5.5 | 5.6 | 5.6 |
| UV (mg/mL) | 19.72 | 18.51 | 18.87 | 18.71 |
| Purity HPSEC | | | | |
| % Monomer | 99.281 | 99.28 | 99.219 | 98.757 |
| % Early Eluting | 0.301 | 0.296 | 0.305 | 0.395 |
| % Late Eluting | 0.419 | 0.425 | 0.476 | 0.849 |
| Purity SDS-PAGE | | | | |
| Reducing (Total | 2.73 | 1.05 | 1.28 | 2.15 |
| Impurity) | | | | |
| Non Reducing | 17.45 | 12.3 | 15.09 | 14.7 |
| (Total Impurity) | | | | |
| Bio Assay | 10.3 mg/mL | 16.46 mg/mL | 20.01 mg/mL | 13.91 mg/mL |
| (SPU/mL) | | | | |
| HIAC | | | | |
| Particle Size (≧10 μm) | 468 | 1161 | 927 | 1069 |
| (Particle | | | | |
| count/container) | | | | |
| Particle Size (≧25 μm) | 30 | 87 | 42 | 71 |
| (Particle | | | | |
| count/container) | | | | |
| Isoelecdtic | Band pattern | Band pattern | Band pattern | Band |
| Focusing (IEF) | matches the | matches the | matches the | pattern |
| | profile of | profile of | profile of | matches |
| | research | research | research batches | the profile |
| | batches | batches | | of research |
| | | | | batches |

| | Sample ID | | | | | |
|---|---|---|---|---|---|---|
| | 3 Month 5 C. | 3 Month 25 C. | 3 Month 40 C. | 3 Month 5 C. | 3 Month 25 C. | 3 Month 40 C. |
| | (Upright) | (Upright) | (Upright) | (Inverted) | (Inverted) | (Inverted) |
| Description | opalescent | opalescent | opalescent | opalescent | opalescent | Opalescent |
| | solution | solution | solution | solution | solution | solution |
| | contains | contains | contains | contains | contains | contains |
| | particles | particles | particles | particles | particles | particles |
| pH | 5.3 | 5.3 | 5.4 | 5.3 | 5.3 | 5.4 |
| UV (mg/mL) | 18.44 | 18.14 | 17.96 | 18.03 | 18.6 | 18.1 |
| Purity HPSEC | | | | | | |
| % Monomer | 99.266 | 99.07 | 97.593 | 99.288 | 98.049 | 97.613 |
| % Early Eluting | 0.301 | 0.335 | 0.691 | 0.3 | 0.339 | 0.7 |
| % Late Eluting | 0.434 | 0.596 | 1.717 | 0.413 | 0.615 | 1.688 |
| Purity SDS-PAGE | | | | | | |
| Reducing (Total | 1.48 | 2.73 | 7.32 | 1.12 | 1.77 | 7.54 |
| Impurity) | | | | | | |
| Non Reducing | 21.17 | 28.13 | 26.67 | 22.64 | 26 | 29.13 |
| (Total Impurity) | | | | | | |
| Bio Assay | 12.93 mg/ml | 15.78 mg/ml | 9.41 mg/ml | 14.13 mg/ml | 13.28 mg/mL | 11.41 mg/ml |
| (SPU/mL) | | | | | | |
| HIAC | | | | | | |
| Particle Size (≧10 μm) | 965 | 532 | 1800 | 586 | 3836 | 322 |
| (Particle | | | | | | |
| count/container) | | | | | | |
| Particle Size (≧25 μm) | 22 | 18 | 185 | 41 | 175 | 10 |
| (Particle | | | | | | |
| count/container) | | | | | | |
| Isoelecdtic | Four to five | Four to five | Four to five | Four to five | Four to five | Four to five |
| Focusing (IEF) | bands | bands between | bands between | bands | bands | bands |
| | between pl | pl markers 8.3 | pl markers 8.3 | between pl | between pl | between pl |
| | markers 8.3 | and 9.5 | and 9.5 | markers 8.3 | markers 8.3 | markers 8.3 |
| | and 9.5 | | | and 9.5 | and 9.5 | and 9.5 |
| | Sample ID | | | | | |
| | 6 Month 5 C. | 6 Month 25 C. | 6 Month 40 C. | 6 Month 5 C. | 6 Month 25 C. | 6 Month 40 C. |
| | (Upright) | (Upright) | (Upright) | (Inverted) | (Inverted) | (Inverted) |
| Description | opalescent | opalescent | opalescent | opalescent | opalescent | opalescent |
| | solution | solution | solution | solution | solution | solution |
| | contains | contains | contains | contains | contains | contains |
| | particles | particles | particles | particles | particles | particles |
| pH | 5.5 | 5.5 | 5.5 | 5.4 | 5.5 | 5.5 |
| UV (mg/mL) | 19.52 | 16.32 | 19.28 | 18.32 | 18.6 | 16.86 |
| Purity HPSEC | | | | | | |
| % Monomer | 99.235 | 98.851 | 95.62 | 99.3 | 98.837 | 95.723 |
| % Early Eluting | 0.25 | 0.317 | 1.406 | 0.229 | 0.319 | 1.348 |
| % Late Eluting | 0.516 | 0.832 | 2.975 | 0.472 | 0.845 | 2.936 |
| Purity SDS-PAGE | | | | | | |
| Reducing (Total | 1.43 | 3.52 | 12.5 | 1.74 | 3.61 | 12.64 |
| Impurity) | | | | | | |
| Non Reducing | 13.67 | 16.55 | 24.86 | 12.68 | 15.64 | 24.33 |
| (Total Impurity) | | | | | | |
| Bio Assay | NA | NA | NA | NA | NA | NA |
| (SPU/mL) | | | | | | |
| HIAC | | | | | | |
| Particle Size (≧10 μm) | 678 | 424 | 1870 | 1894 | 96 | 1270 |
| (Particle | | | | | | |
| count/container) | | | | | | |
| Particle Size (≧25 μm) | 45 | 35 | 90 | 178 | 2 | 78 |
| (Particle | | | | | | |
| count/container) | | | | | | |
| Isoelectric | Four to five | Four to five | Four to five | Four to five | Four to five | Four to five |
| Focusing (IEF) | bands | bands between | bands between | bands | bands | bands |
| | between pl | pl markers 8.3 | pl markers 8.3 | between pl | between pl | between pl |
| | markers 8.3 | and 9.5 | and 9.5 | markers 8.3 | markers 8.3 | markers 8.3 |
| | and 9.5 | | | and 9.5 | and 9.5 | and 9.5 |

### Data Analysis and Reporting

### Batch A

### Description:

The description ranged from clear solution contains particles up to 4 week samples to opalescent solution contains particles for 3 week samples.

pH:

The pH ranged between 5.3 and 5.4.

### UV Conc:

The initial UV concentration obtained was 22.34 mg/mL. The concentration determined by UV assay for the other time points remained constant within 90-110% of the initial value. The differences observed are within the normal variability of this assay.

### HPSEC:

The purity assessed by HPSEC assay suggested that for prototype formulation, the percentage monomer content was more than 99% at 4° C. and 25° C. up to 12 weeks. At 40° C., the percentage monomer content decreased to 98.93 and 98.47 after 2 weeks and 4 weeks respectively.

### SDS-PAGE:

SDS PAGE results suggested typical band pattern which matches with typical non-reducing antibody profile under non-reducing condition and detection of heavy and light chain was reported under reducing condition for all the time points.

### Bioassay:

Bioassay showed significant variability between results of 4 weeks and 12 weeks. The concentration obtained with this assay reduced to 14.0 mg/mL after 2 weeks at 4° C. as compared to initial concentration of 21.4 mg/mL. On the other hand, after 12 weeks at 4° C., the concentration obtained for prototype formulation 1 was 23.3 mg/mL. The differences observed are within the normal variability of this assay.

### HIAC:

The Particulate data met USP <788> specification (Light obscuration test particle count: ≧10 μm-6000 per container, ≧25 μm-600 per container) for all samples.

### Particle Sizing:

The particle size of the samples ranged from 11.05 nm to 14.92 nm for all the samples. The differences observed for particle size are within the normal variability of this assay.

### Batch B

### Description:

The description ranged from clear solution contains particles at initial to opalescent solution contains particles for subsequent samples.

pH:

The pH ranged between 5.3 and 5.5.

### UV Conc:

The initial UV concentration obtained was 19.72 mg/mL. The concentration determined by UV assay for the other time points remained within 90-110% of the initial value. The differences observed are within the normal variability of this assay.

### HPSEC:

The purity assessed by HPSEC assay suggested that for prototype formulation, the percentage monomer content was more than 98% at 4° C. and 25° C. up to 6 months. At 40° C., the percentage monomer content decreased to about 95% after 6 months.

### SDS-PAGE:

Quantitative SDS PAGE results for both reducing and non-reducing conditions show levels of total impurities which remain relatively constant (within the variability of the assay) at 4° C. and 25° C. up to 6 months, with an increase in levels at 40° C. over 6 months.

### Bioassay:

Bioassay showed significant variability over 3 months, with no apparent trend with temperature or time. The differences observed are within the normal variability of this assay.

### HIAC:

The Particulate data met USP <788> specification (Light obscuration test particle count: ≧10 μm-6000 per container, ≧25 μm-600 per container) for all samples.

### Isoelectric Focusing (IEF):

Isoelectric Focusing measures the charge variations in the antibody molecules. The description of the banding pattern reported at Initial and 1 month is equivalent to the description reported at 3 and 6 months, so the results remain constant over 6 months at all temperatures.

### Example 2

### Stability Study of Anti-IGF1R (LCF/HCA) Formulations

The anti-IGF1R antibody used in these studies was the same as that used in Example 1. Based on these studies, the following was determined:
The anti-IGF1R antibody exhibited predominantly β-sheet secondary structure in all the buffers tested.
The anti-IGF1R antibody showed a high Tₒₙₛₑₜ temperature in a pH range of 5 and 6.
The anti-IGF1R antibody, in acetate buffer with pH 5.5, showed highest onset temperatures.
Addition of sodium chloride decreased onset of thermal denaturation for all the buffers tested.
Addition of sucrose increased onset of thermal denaturation for all the buffers tested.
The anti-IGF1R antibody, in a formula of 20 mM acetate buffer pH 5.5 with 7% w/v sucrose, was stable at 4° C. and 25° C. for 28 days.

### Materials.

A stock solution of the anti-IGF1R antibody (28.36 mg/ml) in 5 mM acetate buffer pH5.2 was used to prepare dilutions in various buffers of pH 4 to 9.

TABLE 2
Summary of buffers and pH conditions under which the
anti-IGF1R antibody was formulated.

| Buffers | pH |
|---|---|
| 20 mM acetate | 4, 5, 5.5, 6 |
| 20 mM acetate with NaCl (75 mM or | 5, 5.5 |
| 150 mM) | |
| 20 mM acetate with sucrose (3.5 or | 5, 5.5 |
| 7%) | |
| 20 mM phosphate | 5, 6, 7, 8, 9 |
| 20 mM phosphate with NaCl (75 mM or | 5 |
| 150 mM) | |
| 20 mM phosphate with sucrose (3.5 or | 5 |
| 7%) | |

### Methods.

### Structural Studies

Structural studies were carried out by using circular dichroism (CD). Secondary and tertiary structures were studied by using far UV circular dichroism (FUV) and near UV circular dichroism (NUV) respectively.

### Thermal Denaturation Studies

Protein structural changes were monitored using differential scanning calorimetry (DSC), far UV-circular dichroism spectroscopy (FUV CD), near UV-circular dichroism spectroscopy (NUV CD), tryptophan fluorescence spectroscopy (TRP FL), and particle size by light scattering (PS) as the samples were heated at a constant rate.

### Short Term Stability Studies

Real time stability of the antibody was studied in 20 mM acetate buffer pH 5.5 with sucrose. The stability conditions used were 4, 25 and 40° C. and the samples were kept for 1 month. The percentage monomer content was analyzed by using HPSEC assay.

### Results and Discussion.

Far UV (FUV) circular dichroism scan in acetate buffer at pH5. A minimum of 217 nm and shoulder at 235 nm indicate the predominant presence of β-sheet secondary structure. Maximum at 202 nm is due to presence of β-turn secondary structures (see FIG. 1(a)).

Near UV (NUV) circular dichroism scan in acetate buffer at pH5. Near UV CD spectrum shows three distinct regions:
250-270 nm: phenylalanine residues,
270-290 nm: tyrosine residues,
280-300 nm: tryptophan residues (see FIG. 1(b)).

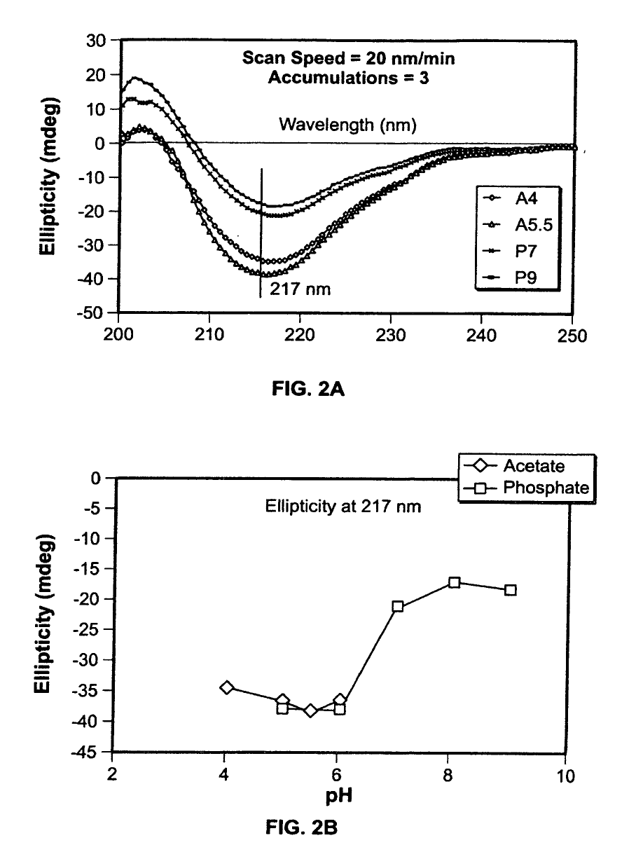

Far UV (FUV) circular dichroism scan in various buffers. As shown in FIG. 2(a), change in ellipticity with pH was observed at 217 nm, 235 nm and 202 nm. The minimum values of ellipticity corresponding to β-sheet secondary structure was observed between pH 5 and 6.

Changes in ellipticity as a function of pH. For pH above 6, ellipticity increases signifying structural change in β-sheet secondary structure (FIG. 2(b)). Similar trend was observed at 235 nm (FIG. 2(c)). Ellipticity at 202 nm increases above pH 6, which suggests an increase in β-turn secondary structure (FIG. 2(d)).

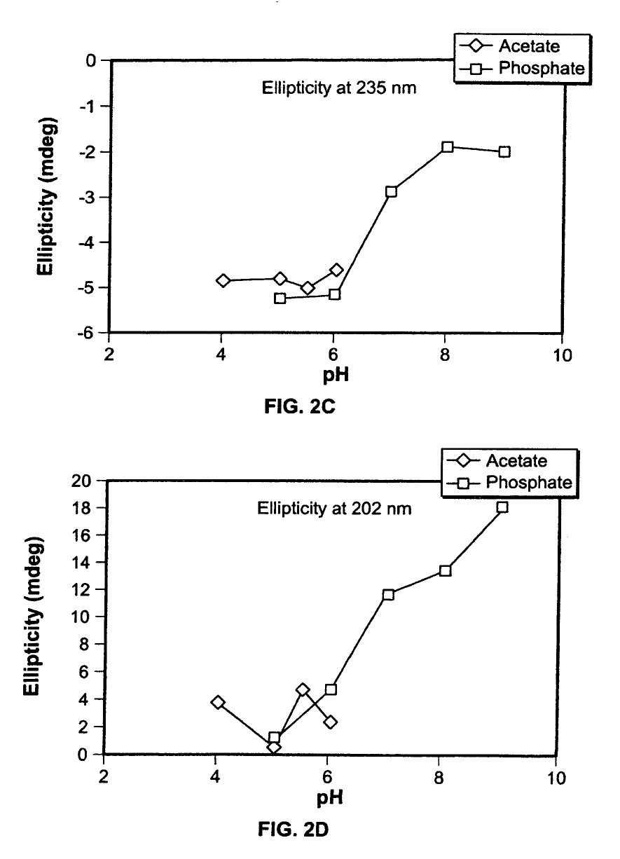

Near UV (FUV) circular dichroism scan in various buffers. No appreciable change in tertiary structure was observed (see FIG. 3).

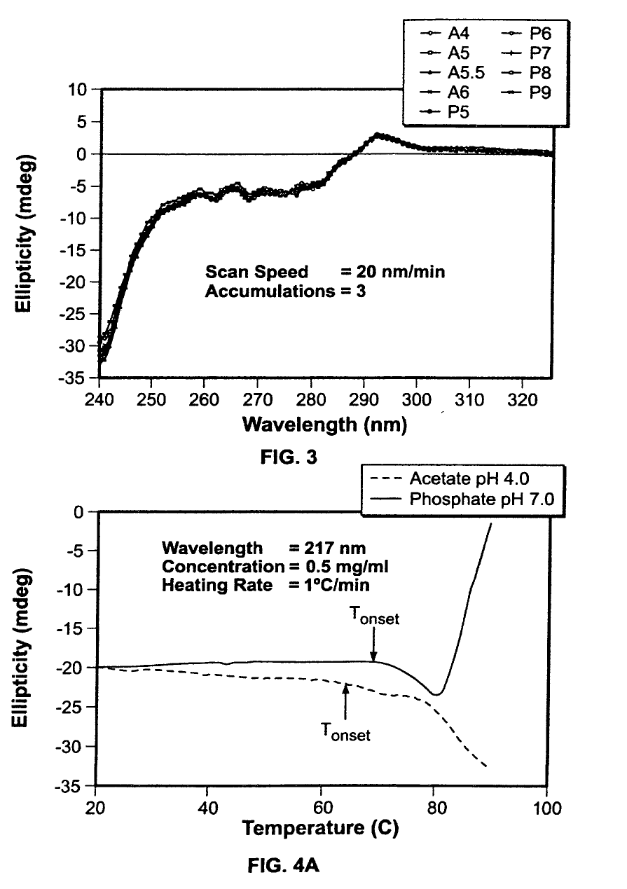

Thermal studies. On heating samples from 20-63° C. no change was seen in the CD signal of the anti-IGF1R antibody signifying no change in the secondary structure in either buffer. At Tₒₙₛₑₜ (64.1° C., pH 4) a decrease in CD signal was seen due to unfolding and change in secondary structure. The ellipticity further increased with increase in temperature possibly due to formation of intermolecular β-sheet secondary structure in aggregates. The anti-IGF1R antibody in phosphate buffer at pH 7 showed Tₒₙₛₑₜ at 68.3° C. At 80° C., an decrease in ellipticity was observed possibly due to precipitation and loss of the anti-IGF1R antibody in solution. Acetate buffer at pH 5.5 depicted highest onset temperature compared to other buffers. See FIG. 4.

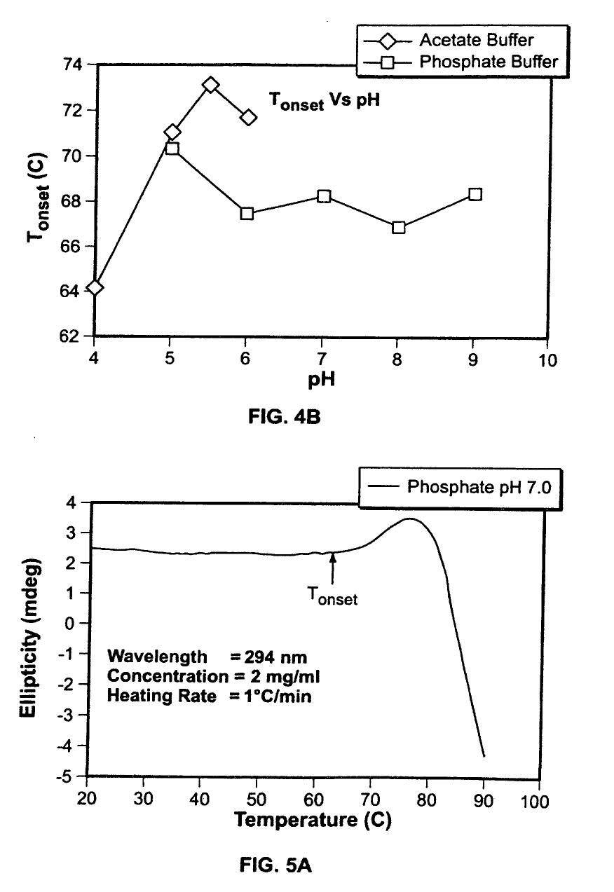

On heating the anti-IGF1R antibody samples from 20-60° C., ellipticity by NUV CD remained constant at 294 nm (FIG. 5(a)). At 61° C., an increase in the ellipticity can be seen which was followed by a decrease in ellipticity suggesting local changes in tryptophan environment due to unfolding of protein. Tₒₙₛₑₜ temperatures for acetate buffer at pH 5.5 and 6 were higher than that seen for other buffers (FIG. 5(b)).

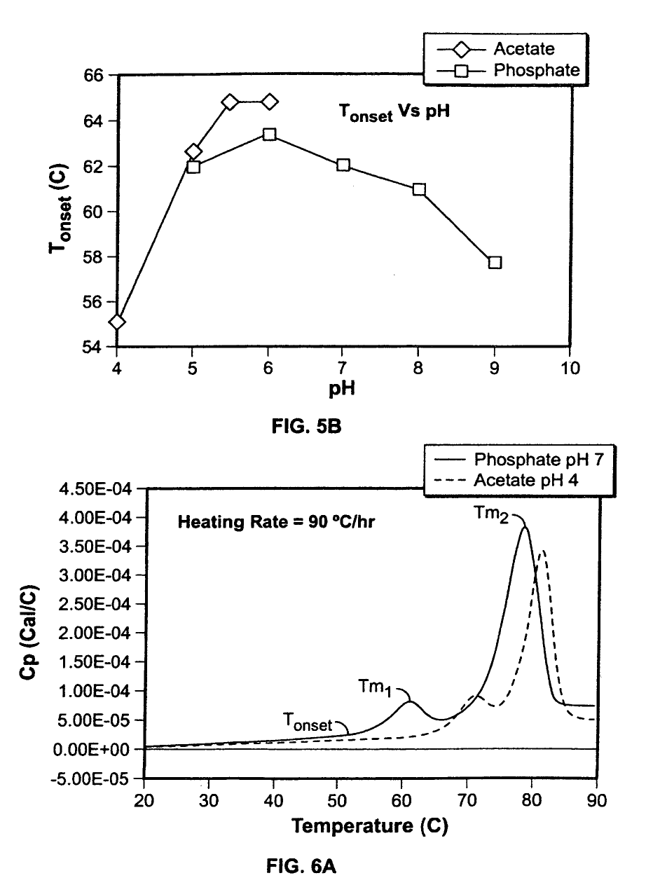

DSC thermograms showed two transition temperatures, Tₘ₁ and Tₘ₂ (FIG. 6(a)). These are the temperatures at which maximum enthalpy change occurs due to protein structural change. Highest Tₒₙₛₑₜ temperature was observed in acetate buffer at pH 5.5 (FIG. 6(b)). Acetate buffer at pH 6 showed highest Tₘ₁ at 69.9° C. (FIG. 6(c)) while acetate buffer at pH 5.5 and 6.0 depicted highest Tₘ₂ at 82.2 and 82.3° C. respectively (not shown).

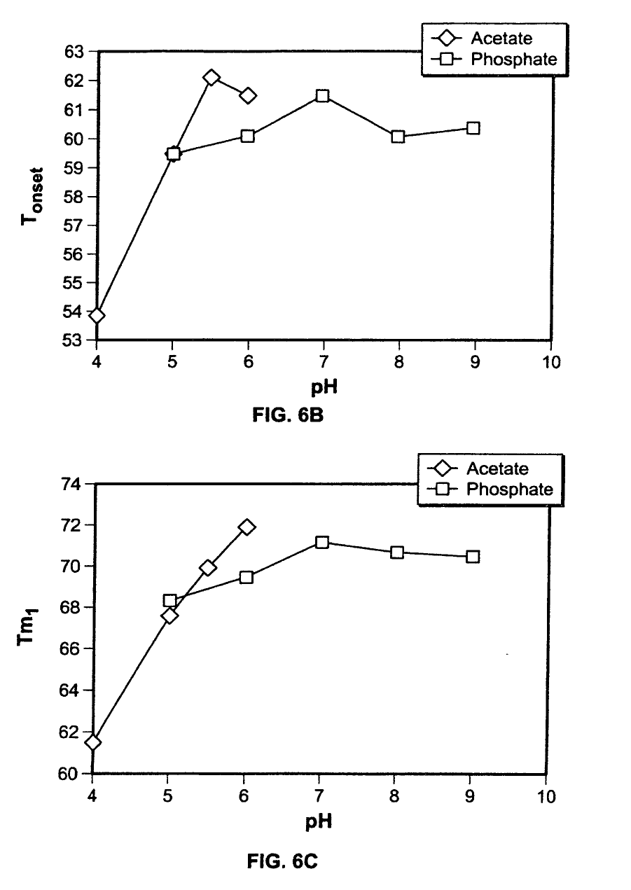

Particle size/aggregation studies. FIG. 7(a) shows particle size distribution obtained for the anti-IGF1R antibody. Mean size of anti-IGF1R antibody in all the buffers tested was 11.05 nm. FIG. 7(b) shows the change in size distribution of anti-IGF1R antibody at various temperatures. As temperature increases, increase in size can be observed due to aggregate formation.

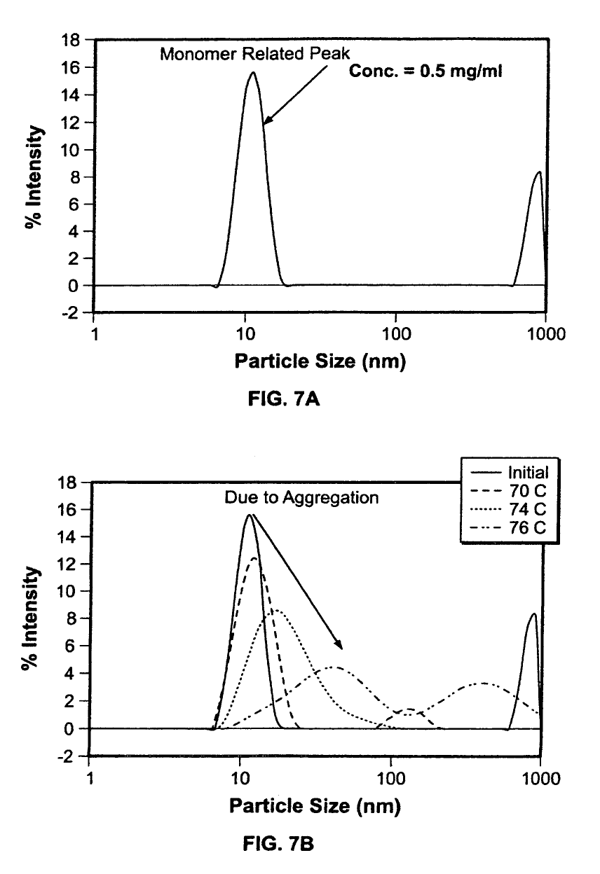

Phosphate buffer at pH 5 showed highest Tₒₙₛₑₜ of aggregation at 76° C. Acetate buffers at pH 5, 5.5 and 6 showed Tₒₙₛₑₜ of aggregation at 74° C. while remaining buffers showed aggregation at 70° C. (see FIG. 8(a)). Tₒₙₛₑₜ of aggregation was not observed in acetate buffer at pH 4 (see FIG. 8(b)).

TABLE 3
Summary of thermal melt data obtained by various techniques.

| Buffer | TRP FL | FUV CD | NUV CD | DSC | | | PS |
|---|---|---|---|---|---|---|---|
| Solution | Tₒₙₛₑₜ | Tₒₙₛₑₜ | Tₒₙₛₑₜ | Tₒₙₛₑₜ | Tₘ₁ | Tₘ₂ | Tₘ |
| Ace 4.0 | 63.9 | 64.1 | 55.0 | 53.8 | 61.4 | 78.8 | — |
| Ace 5.0 | 64.9 | 71.1 | 62.7 | 59.6 | 67.6 | 81.1 | 74.0 |
| Ace 5.5 | 68.4 | 73.2 | 64.8 | 62.2 | 69.9 | 82.2 | 74.0 |
| Ace 6.0 | 62.9 | 71.8 | 64.8 | 61.6 | 71.9 | 82.3 | 74.0 |
| Phos 5.0 | 60.4 | 70.4 | 62.0 | 59.5 | 61.3 | 81.8 | 76.0 |
| Phos 6.0 | 61.4 | 67.6 | 63.4 | 60.2 | 69.4 | 82.2 | 74.0 |
| Phos 7.0 | 61.9 | 68.3 | 62.0 | 61.5 | 71.2 | 81.5 | 70.0 |
| Phos 8.0 | 60.9 | 66.9 | 61.0 | 60.1 | 70.7 | 80.8 | 70.0 |
| Phos 9.0 | 60.0 | 68.3 | 57.6 | 60.4 | 70.4 | 80.7 | 70.0 |

The anti-IGF1R antibody exhibited higher Tₒₙₛₑₜ and Tₘ in the pH region of 5 and 6. Most techniques showed higher Tₒₙₛₑₜ and Tₘ in acetate buffer at pH 5.5.

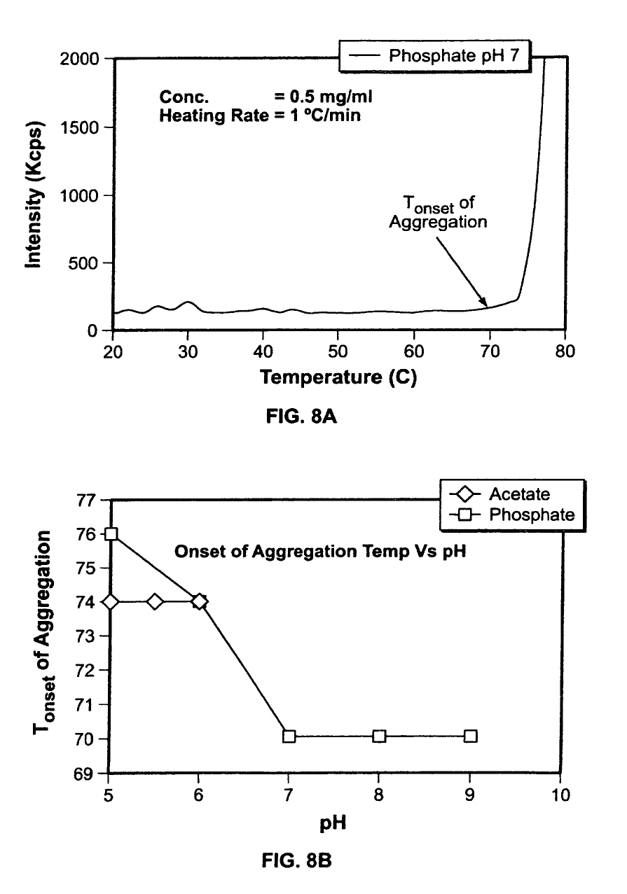

Effect of NaCl or sucrose on Tₒₙₛₑₜ. The addition of sodium chloride decreased FUV CD Tₒₙₛₑₜ temperatures indicating that protein unfolding occurs at lower temperature. Similar trends were seen when the effect of sodium chloride on the anti-IGF1R antibody was studied using NUV CD, TRP FL, PS and DSC. See FIG. 9.

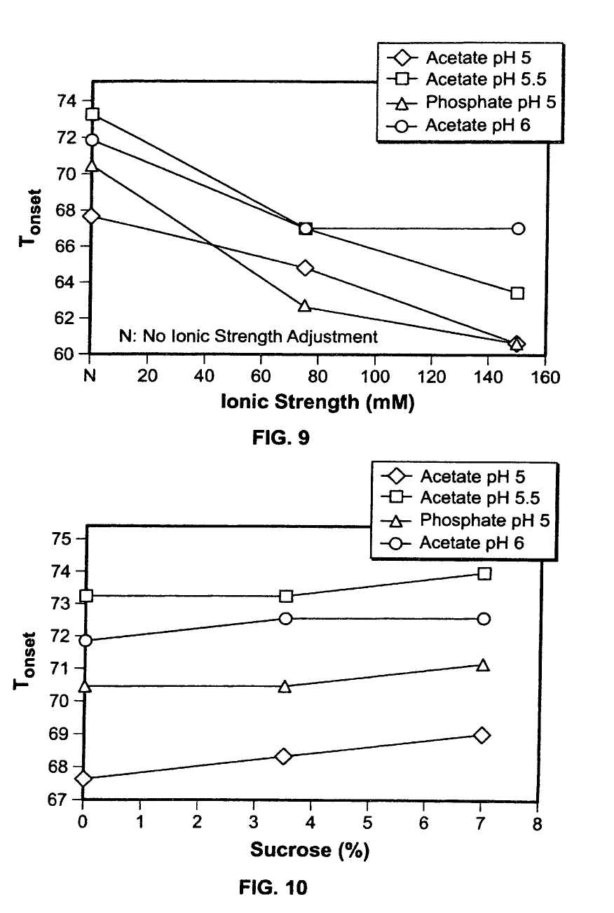

The addition of sucrose increased FUV CD Tₒₙₛₑₜ temperatures indicating that protein unfolding occurs at higher temperature. Similar trends were seen when the effect of sucrose on the anti-IGF1R antibody was studied using NUV CD, TRP FL, PS and DSC. See FIG. 10.

These experiments demonstrated that sucrose had a stabilizing effect on the anti-IGF1R antibody.

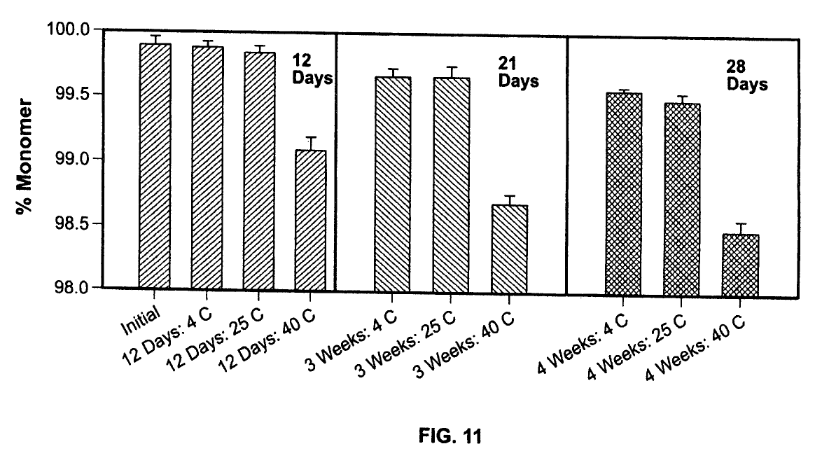

Stability study of the anti-IGF1R antibody in acetate buffer, 7% sucrose and pH5.5. The anti-IGF1R antibody (15 mg/ml) in 20 mM acetate buffer at pH 5.5 with 7% w/v sucrose was placed on stability at 4° C., 25° C. and 40° C. After 12 days, the monomer content for 40° C. decreased to 99%. The monomer content at 4° C. and 25° C. were comparable to initial. After 21 and 28 days, monomer content for 40° C. sample decreased to 98.7% and 98.5%, respectively. At 4 and 25° C., monomer content dropped slightly (approximately 0.2%) compared to initial. See FIG. 11.

### Example 3

### Efficacy of LCF/HCA in Mouse Tumor Models at a Blood Concentration of 19 μg/mL

In order to determine the trough therapeutic serum concentration of the LCF/HCA formulation described above for treating IGF-1R mediated disorders, the following efficacy study was conducted in a human non-small cell lung cancer xenograft model. Twenty 6-8 week old female nude mice were used in the study. Four million H322 cells, mixed 1:1 with Matrigel, were inoculated subcutaneously into the flank of each mouse. Dosing was initiated when the tumors reached an average size of 110 mm³.

TABLE 4
Xenograph Study Design

| | Mouse | Number and | Dosing | |
|---|---|---|---|---|
| Groups | strain | Sex of Mice | Schedule | Route |
| IgG Control | Nude | 10 female | 2x/wk | IP |
| 0.1 mg LCF/HCA | Nude | 10 female | 2x/wk | IP |

In the first study, a single 0.1 mg dose of LCF/HCA was administered IP to each mouse. The concentration of LCF/HCA was measured at day 3 after the 1^{st} dose. In a second H322 study, the LCF/HCA concentration in plasma was determined at day 21, after five doses. The results are summarized in Table 5 below:

TABLE 5

| | % TGI (tumor growth | |
|---|---|---|
| | inhibition) at 0.1 mg/ | |
| H322 | mouse | LCF/HCA (nM) |
| Study 1 | 68% | 131.5 (average of two |
| | | measurements: 69 and 194) |
| Study 2 | 71% | 132 |

In both studies, significant tumor growth inhibition was observed at approximately 130 nM serum concentration, establishing the minimal therapeutic concentration at 19 μg/mL (nM=μg/MW/mL×100,000; For a full length antibody, the average molecular weight is 150,000 Daltons).

A 19 μg/mL blood concentration was also observed to inhibit a human neuroblastoma xenograft model (using cell line SK-N-AS). The above-referenced xenograft studies were performed using LCF/HCA antibody with an IgG1 constant region. Additional studies were performed using LCF/HCA bound to an IgG4 constant region wherein the 19 μg/mL blood concentration was observed to be effective at inhibiting a human ovarian cancer xenograft model (using cell line A27A0).

A mouse xenograft study using human colorectal cells (cell line HT29) was also carried out using LCF/HCA with an IgG1 constant region. In this study, the 0.1 mg dose in mice led to a blood concentration of about 38 μg/mL; this concentration of the antibody was also observed to be effective at inhibiting growth of the colorectal cell line.

### Example 4

### Pharmacokinetic Study of LCF/HCA (IgG1) in Cynomolgus Monkeys

In this example, the pharmacokinetics of LCF/HCA following a single intravenous dose to male cynomolgus monkeys were evaluated. Each monkey received single intravenous injections of 1, 3, 4 or 30 mg/kg LCF/HCA. The samples were assayed by a non-validated enzyme-linked immunoabsorbent assay (ELISA) to determine LCF/HCA concentrations in cynomolgus monkey serum. Serum samples were qualitatively analyzed for anti-IGF1R LCF/HCA antibodies using a biosensor-based assay. Study results are described in Tables 6-9 below.

TABLE 6
Individual and Mean (% CV) Serum Concentrations for LCF/HCA
Following a Single 1 mg/kg IV Dose

| | Monkey | | Monkey | | Monkey | | | |
|---|---|---|---|---|---|---|---|---|
| Time | 103 | | 299 | | 400 | | Mean^{a} | |
| (day) | μg/Ml | Antibody^{c} | μg/mL | Antibody | μg/mL | Antibody | (μg/mL) | CV^{b} |
| Pre-dose | 0 | negative | 0 | negative | 0 | negative | 0 | NC |
| 0.014 | 24.591 | NT^{d} | 19.733 | NT | 27.543 | NT | 23.956 | 16.5 |
| 0.042 | 26.155 | NT | 18.831 | NT | 30.232 | NT | 25.073 | 23.0 |
| 0.083 | 26.451 | NT | 18.598 | NT | 34.991 | NT | 26.680 | 30.7 |
| 0.17 | 20.693 | NT | 17.877 | NT | 33.509 | NT | 24.026 | 34.7 |
| 0.25 | 19.935 | NT | 17.442 | NT | 28.653 | NT | 22.010 | 26.7 |
| 0.42 | 20.030 | NT | 11.672 | NT | 26.089 | NT | 19.264 | 37.6 |
| 1 | 16.100 | NT | 16.909 | NT | 24.867 | NT | 19.292 | 25.1 |
| 2 | 7.858 | NT | 6.581 | NT | 13.459 | NT | 9.299 | 39.3 |
| 3 | 5.448 | NT | 4.986 | NT | 11.822 | NT | 7.419 | 51.5 |
| 4 | 4.350 | NT | 4.175 | NT | 9.228 | NT | 5.918 | 48.5 |
| 7 | 1.798 | negative | 1.007 | negative | 4.847 | negative | 2.551 | 79.5 |
| 9 | 0.226^{e} | negative | 0 | positive | 2.239 | negative | 0.822 | 150 |
| 14 | 0 | negative | 0 | positive | 0.022^{e} | negative | 0.007 | NC^{f} |
| 21 | 0 | negative | 0 | positive | 0 | negative | 0 | NC |
| 28 | 0 | negative | 0 | positive | 0 | negative | 0 | NC |

^{a}n = 3
^{b}Coefficient of variation expressed as a percent
^{c}Anti-LCF/HCA antibody assay
^{d}Not tested (NT)
^{e}Data excluded from slope used for half-life determination, see text for discussion
^{f}Not calculated (NC)

TABLE 7
Individual and Mean Serum Concentrations for LCF/HCA
Following a Single 3 mg/kg IV Dose

| | Monkey | | Monkey | | |
|---|---|---|---|---|---|
| | 303 | | 603 | | Mean^{a} |
| Time (day) | μg/mL | Antibody^{b} | μg/mL | Antibody | (μg/mL) |
| Pre-dose | 0 | negative | 0 | negative | 0.000 |
| 0.014 | 57.783 | NT^{c} | 54.848 | NT | 56.316 |
| 0.042 | 63.683 | NT | 63.739 | NT | 63.711 |
| 0.083 | 59.141 | NT | 52.964 | NT | 56.053 |
| 0.17 | 68.266 | NT | 53.427 | NT | 60.847 |
| 0.25 | 52.185 | NT | 55.174 | NT | 53.680 |
| 0.42 | 48.146 | NT | 60.907 | NT | 54.527 |
| 1 | 45.000 | NT | 50.905 | NT | 47.953 |
| 2 | 35.061 | NT | 32.957 | NT | 34.009 |
| 3 | 26.140 | NT | 24.678 | NT | 25.409 |
| 4 | 24.622 | NT | 24.160 | NT | 24.391 |
| 7 | 15.365 | negative | 15.022 | negative | 15.194 |
| 9 | 10.893 | negative | 10.038 | negative | 10.466 |
| 14 | 0 | positive | 0.015^{d} | negative | 0.008 |
| 21 | 0 | positive | 0 | positive | 0.000 |
| 28 | 0 | positive | 0 | positive | 0.000 |

^{a}n = 2
^{b}Anti-LCF/HCA antibody assay
^{c}Not tested (NT)
^{d}Data excluded from slope used for half-life determination, see text for discussion.

TABLE 8
Individual Serum Concentrations for
LCF/HCA Following a Single 4 mg/kg IV Dose

| | Monkey | |
|---|---|---|
| Time | 703 | |
| (day) | μg/mL | Antibody^{a} |
| Pre-dose | 0 | negative |
| 0.014 | 97.057 | NT |
| 0.042 | 104.745 | NT |
| 0.083 | 101.677 | NT |
| 0.17 | 90.061 | NT |
| 0.25 | 91.971 | NT |
| 0.42 | 93.050 | NT |
| 1 | 80.691 | NT |
| 2 | 31.637 | NT |
| 3 | 28.708 | NT |
| 4 | 31.819 | NT |
| 7 | 21.752 | negative |
| 9 | 9.034 | negative |
| 14 | 0 | negative |
| 21 | 0 | positive |
| 28 | 0 | positive |

^{a}Anti-LCF/HCA antibody assay

TABLE 9
Individual and Mean (% CV) Serum Concentrations for LCF/HCA
Following a Single 30 mg/kg IV Dose

| | Monkey | | Monkey | | Monkey | | | |
|---|---|---|---|---|---|---|---|---|
| Time | 203 | | 503 | | 1002 | | Mean^{a} | |
| (day) | μg/mL | Antibody^{c} | μg/mL | Antibody | μg/mL | Antibody | (μg/mL) | CV^{b} |
| Pre-dose | 14 | negative | 0 | negative | 0 | negative | 0 | NC |
| 0.014 | 782.490 | NT^{d} | 674.061 | NT | 902.979 | NT | 786.510 | 14.6 |
| 0.042 | 854.041 | NT | 802.923 | NT | 956.872 | NT | 871.279 | 9.0 |
| 0.083 | 851.958 | NT | 575.333 | NT | 751.937 | NT | 726.409 | 19.3 |
| 0.17 | 751.381 | NT | 518.323 | NT | 620.985 | NT | 630.230 | 18.5 |
| 0.25 | 721.226 | NT | 514.296 | NT | 601.335 | NT | 612.286 | 17.0 |
| 0.42 | 668.567 | NT | 459.046 | NT | 745.556 | NT | 624.390 | 23.7 |
| 1 | 609.460 | NT | 400.111 | NT | 617.047 | NT | 542.206 | 22.7 |
| 2 | 344.272 | NT | 261.217 | NT | 377.721 | NT | 327.737 | 18.3 |
| 3 | 276.557 | NT | 243.272 | NT | 296.595 | NT | 272.141 | 9.9 |
| 4 | 286.680 | NT | 266.381 | NT | 285.683 | NT | 279.581 | 4.1 |
| 7 | 216.866 | negative | 207.064 | negative | 205.962 | negative | 209.964 | 2.9 |
| 9 | 135.445 | negative | 122.640 | negative | 112.079 | negative | 123.388 | 9.5 |
| 14 | 100.142 | negative | 111.141 | negative | 72.542 | negative | 94.608 | 21.0 |
| 21 | 78.199 | negative | 75.342 | negative | 50.923 | negative | 68.155 | 22.0 |
| 28 | 32.168 | negative | 33.735 | negative | 22.843 | negative | 29.582 | 19.9 |
| 11^{e} | NT | negative | NT | negative | NT | negative | | |
| 15^{e} | NT | negative | NT | negative | NT | negative | | |

^{a}n = 3
^{b}Coefficient of variation expressed as a percent
^{c}LCF/HCA antibody assay
^{d}Not tested (NT)
^{e}week

### Example 5

### Toxicity and Toxicokinetic Study of LCF/HCA in Cynomolgus Monkeys

### Study Design

The toxicokinetics of the LCF/HCA antibody formulation described above were evaluated in cynomolgus monkeys undergoing a three-month toxicity study with a four-month postdose period. Each monkey received single intravenous (bolus) injections of 10, 30 or 100 mg/kg LCF/HCA once every seven days for three months (a total of 13 injections). Blood samples were obtained from all monkeys prior to dosing and at 1, 8, 24, 48, 72, 96 and 168 hr after dosing (target time points) on Days 0 and 84. In addition, samples were collected from all monkeys prior to dosing and at 1 hr after dosing (target time point) on Days 14, 28, 42, 56 and 70. In addition, single samples were collected from monkeys assigned to the four-month postdose period on Days 94, 98, 112, 126, 140, 154, 168, 182, 196 and 207. Dosing Interval 1 contains sampling Days 0 through 7, and Dosing Interval 12 includes sampling Days 84 through 91. Serum concentrations of LCF/HCA were determined using a validated electrochemiluminescence (ECL)-based immunoassay. The presence of antibodies against LCF/HCA were determined using a validated ECL-based immunoassay. Samples that were positive for antibodies against LCF/HCA were assayed for the presence of neutralizing antibodies using a non-validated Kinase Receptor Activation (KIRA) assay. The toxicokinetic parameters of LCF/HCA were estimated from individual serum concentration-time profiles.

### Toxicokinetic Analysis

Serum concentrations of LCF/HCA were analyzed using model-independent methods (Gibaldi et al., Pharmacokinetics. 2d ed. Marcel Decker, Inc., NY (1982), pp. 409-417). The following LCF/HCA toxicokinetic parameters were obtained for each animal for Dosing Interval 1 (Days 0-7) and Dosing Interval 12 (Days 84-91): concentration at time zero (C0), maximum observed serum concentration (Cmax), time of maximum observed serum concentration (Tmax), and area under the serum concentration-time curve (AUC). Vd(initial) was calculated for each animal for Dosing Interval 1 only. Half-life (t½), clearance at steady state (Clss), volume of distribution at steady state (Vss) and mean residence time from the time of dosing to infinity [MRT(I)] were calculated for the animals assigned to the four-month postdose period from Days 84 to 207. Concentrations less than the lower limit of quantitation (LLOQ) were reported as and set to zero in the calculations. AUC and C0 values were calculated only for animals that had at least four consecutive quantifiable time points. AUC and C0 values were not determined for Dosing Interval 12 (Days 84-91) for animal Nos. 1005 (Male) and 1505 (Female) due to insufficient serum concentration data (less than four consecutive quantifiable time points).

C0 was determined by log-linear regression analysis using the LCF/HCA serum concentration values at the first two time points, where the y intercept equaled C0 (only when the regression analysis yielded a slope<0). In cases for which the regression analysis yielded a slope≧0, the first observed serum concentration was used as an estimate for C0. The AUC values from time 0 to 7 days [AUC(0-7 days)] for Dosing Interval 1 (Days 0-7) and Dosing Interval 12 (Days 84-91) were calculated by the linear trapezoidal method. The initial volume of distribution was calculated as Dose/C0. Half-life was calculated as (ln 2)/K. Clearance at steady state was calculated as Dose/AUC^{τ}_{O} (τ=7 days). The volume of distribution at steady state was calculated as MRT(I)*Clₛₛ. Mean residence time from the time of dosing to infinity was calculated as the AUMC(I)÷AUC(I). The AUMC(I) and AUC(I) values were calculated by the linear trapezoidal method. The accumulation ratio, R, was calculated as:

R=AUC_{Dosing Interval 12}÷AUC_{Dosing Interval 1}

### Computer Software

Pharsight® Knowledgebase Server™: version 2.0.1 with WinNonlin version 4.0.1 (Pharsight Corporation, Cary, N.C.) was used to conduct the pharmacokinetic analysis. Excel 2002 (Microsoft Corporation, Redmond, Wash.) was used for the control animal serum concentration assessment.

### Results

TABLE 10
Mean (CV) LCF/HCA toxicokinetic parameters for dosing
interval 1 (days 0-7) and dosing interval 12 (days 84-91)
following intravenous bolus administration of 10, 30 or
100 mg/kg LCF/HCA to cynomolgus monkeys
(males and females combined).

| | | | AUC | | |
|---|---|---|---|---|---|
| Dose | Dosing | C₀ | (0-7 days) | Vd (initial) | |
| (mg/kg) | Interval | (μg/mL) | (μg · day/mL) | (mL/kg) | R^{b} |
| 10 | 1 | 322 (10) | 819 (10) | 31.4 (10) | NA^{c} |
| | 12^{d} | 577 (21) | 1580 (13) | NA | 1.97 (16) |
| 30 | 1 | 956 (11) | 3120 (11) | 31.7 (9) | NA |
| | 12 | 2310 (23) | 5580 (13) | NA | 1.79 (11) |
| 100 | 1 | 4270 (24) | 12600 (14) | 24.5 (21) | NA |
| | 12 | 5060 (17) | 18000 (9) | NA | 1.45 (17) |

^{a}N = 12/dose group/dosing interval, unless otherwise noted
^{b}R = AUC (0-7 days)_{Dosing Interval 12} ÷ AUC (0-7 days)_{Dosing Interval 1}
^{c}NA = Not applicable
^{d}N = 10

TABLE 11
Mean (CV) LCF/HCA toxicokinetic parameters for animals from
the four month postdose period (days 84-207) following
intravenous bolus administration of 10, 30 or 100 mg/kg LCF/HCA
to cynomolgus monkeys (males and females combined).

| | t½ | CIₛₛ | Vₛₛ | MRT(I) |
|---|---|---|---|---|
| Dose (mg/kg) | (day) | (mL/day/kg) | (mL/kg) | (day) |
| 10 | 9.43 (27) | 6.32 (17) | 74.3 (11) | 11.9 (12) |
| 30 | 10.5 (29) | 5.48 (12) | 60.8 (12) | 11.3 (19) |
| 100 | 11.6 (21) | 5.62 (12) | 75.4 (22) | 13.4 (17) |

a: N = 6/dose group

TABLE 12
Mean (CV) LCF/HCA serum concentrations for dosing interval 1
(days 0-7) and dosing interval 12 (days 84-91) following
intravenous bolus administration of 10, 30 or 100 mg/kg
LCF/HCA to male and female cynomolgus monkeys.

| Dose | Time [symbol] | Dosing Interval 1 | | | | Dosing Interval 12 | | | |
|---|---|---|---|---|---|---|---|---|---|
| (mg/kg) [symbol] | (Day) | Female | | Male | | Female | | Male | |
| 10 | 1 [symbol] | 308 [symbol] | (11) | 303 | (10) | 471 | (50) | 414 | (55) |
| | 8 [symbol] | 216 | (10) | 209 | (10) | 245 | (51) | 241 | (49) |
| | 1 | 179 | (10) | 183 | (12) | 191 | (51) | 187 | (49) |
| | 2 | 146 | (10) | 129 | (10) | 155 | (55) | 161 | (49) |
| | 3 | 97.0 | (19) | 95.5 | (12) | 238 | (51) | 248 | (51) |
| | 4 | 94.0 | (11) | 85.1 | (14) | 138 | (52) | 160 | (50) |
| | 7 | 58.7 | (17) | 69.3 | (11) | 165 | (58) | 180 | (52) |
| 30 | 1 [symbol] | 891 | (7) | 895 | (12) | 2280 | (23) | 1960 | (12) |
| | 8 [symbol] | 572 | (13) | 538 | (7) | 1140 | (10) | 1240 | (14) |
| | 1 | 777 | (33) | 951 | (31) | 774 | (9) | 785 | (13) |
| | 2 | 485 | (10) | 442 | (12) | 630 | (9) | 651 | (15) |
| | 3 | 417 | (12) | 364 | (12) | 840 | (14) | 921 | (24) |
| | 4 | 422 | (10) | 351 | (15) | 651 | (9) | 793 | (23) |
| | 7 | 221 | (21) | 230 | (12) | 840 | (12) | 680 | (25) |
| 100 | 1 [symbol] | 4070 | (22) | 4090 | (25) | 4350 | (12) | 5370 | (10) |
| | 8 [symbol] | 3250 | (35) | 2940 | (35) | 3700 | (11) | 3780 | (11) |
| | 1 | 2350 | (13) [symbol] | 2440 | (23) | 2840 | (5) | 2800 | (5) |
| | 2 | 1850 | (23) | 1950 | (22) | 2510 | (19) | 2440 | (7) |
| | 3 | 1600 | (6) | 1720 | (12) | 2780 | (10) | 3310 | (7) |
| | 4 | 1160 | (25) | 1120 | (48) | 2170 | (15) | 2280 | (13) |
| | 7 | 1400 | (27) | 1840 | (14) | 1500 | (47) | 2360 | (8) |

a: N = 6/sex/dose group, unless otherwise noted
b: time units are in days, unless otherwise noted
c: time units in hours
d: LCF/HCA serum concentration units are mcg/ml
e: N = 5 [symbol]
indicates data missing or illegible when filed

TABLE 13
Mean (CV) LCF/HCA serum concentrations for animals from the four
month postdose period (days 84-207) following intravenous bolus administration of
10, 30 or 100 mg/kg LCF/HCA to male and female cynomolgus monkeys.

| | [symbol] Dose (mg/kg) [symbol] | | | | | | | | | | | |
|---|---|---|---|---|---|---|---|---|---|---|---|---|
| Time [symbol] | 10 | | | | 30 | | | | 100 | | | |
| (day) | Female | | Male | | Female | | Male | | Female | | Male | |
| 1 [symbol] | 573 [symbol] | (13) | 542 | (26) | 2310 | (31) | 1900 | (17) | 4230 | (7) | 5650 | (7) |
| 8 [symbol] | 300 | (17) | 299 | (3) | 1100 | (9) | 1290 | (10) | 3790 | (14) | 4000 | (6) |
| 1 | 233 | (15) | 227 | (3) | 728 | (7) | 733 | (12) | 2880 | (12) | 2940 | (7) |
| 2 | 190 | (24) | 198 | (4) | 905 | (10) | 654 | (14) | 2550 | (14) | 2430 | (9) |
| 3 | 291 | (18) | 312 | (13) | 902 | (18) | 955 | (29) | 2310 | (7) | 3320 | (9) |
| 4 | 175 | (28) | 199 | (6) | 916 | (6) | 652 | (23) | 2290 | (16) | 2350 | (9) |
| 7 | 199 | (29) | 211 | (7) | 638 | (19) | 625 | (23) | 1270 | (79) | 2250 | (4) |
| 10 | 116 | (25) | 123 | (6) | 321 | (20) | 356 | (27) | 1350 | (17) | 1370 | (13) |
| 14 | 83.2 | (29) | 59.8 | (17) | 254 | (22) | 265 | (39) | 1040 | (18) | 1190 | (14) |
| 28 | 23.9 | (50) | 27.8 | (33) | 37.1 | (36) | 112 | (56) | 288 | (43) | 399 | (31) |
| 42 | 8.75 | (127) | 11.4 | (84) | 29.9 | (50) | 38.0 | (50) | 145 | (53) | 210 | (45) |
| 58 | 0.994 | (84) | 1.52 | (68) | 5.57 | (50) | 18.2 [symbol] | (NR) | 56.4 | (80) | 97.7 | (49) |
| 70 | 0.594 | (88) | 1.09 | (51) | 3.22 | (47) | 7.52 [symbol] | (NR) | 19.5 | (93) | 45.5 | (91) |
| 84 | 0.219 | (112) | 0.445 | (58) | 1.84 | (84) | 5.31 [symbol] | (NR) | 10.5 | (103) | 20.4 | (95) |
| 93 | 0.162 | (84) | 0.304 | (65) | 0.711 | (50) | 2.82 [symbol] | (NR) | 4.79 [symbol] | (NR) | 13.5 | (80) |
| 112 | 0.138 | (100) | 0.0770 [symbol] | (NR) | 0.528 | (80) | 2.25 [symbol] | (NR) | 1.97 [symbol] | (NR) | 7.33 | (80) |
| 123 | 0.0640 | (107) | 0.0880 | (129) | 0.317 [symbol] | (NR) | 1.59 [symbol] | (NR) | 2.19 | (88) | 3.04 | (22) |

a: N = 3/sex/dose group, unless otherwise noted
b: time units are in days (following the final dose on day 84), unless otherwise noted
c: time units are hours
d: LCF/HCA serum concentration units are mcg/ml
e: N = 2
f: NR = not reporter when N < 3 [symbol]
indicates data missing or illegible when filed

TABLE 14
Mean (CV) LCF/HCA toxicokinetic parameters for dosing interval 1 (days 0-7)
and dosing interval 12 (days 84-91) following intravenous bolus administration of
10, 30 or 100 mg/kg LCF/HCA to male and female cynomolgus monkeys.

| [symbol] | Dosing | | C [symbol] | AUC (0-7 days) | Vd (initial) | |
|---|---|---|---|---|---|---|
| Dose (mg/kg) | Interval | Sex | (μg/mL) | (μg day/mL) | (mL/kg) | R [symbol] |
| 10 | 1 | F | 325 (12) | 846 (8) | 31.2 (12) | NA [symbol] |
| | | M | 319 (10) | 791 (11) | 31.9 (10) | NA |
| | 12 [symbol] | F | 619 (13) | 1530 (19) | NA | 1.84 (15) |
| | | M | 538 (27) | 1620 (6) | NA | 2.09 (16) |
| 30 | 1 | F | 950 (7) | 3170 (12) | 31.7 (6) | NA |
| | | M | 963 (14) | 3080 (11) | 31.8 (12) | NA |
| | 12 | F | 2530 (27) | 5350 (6) | NA | 1.71 (10) |
| | | M | 2100 (13) | 5800 (15) | NA | 1.88 (11) |
| 100 | 1 | F | 4250 (25) | 12400 (13) | 24.6 (22) | NA |
| | | M | 4300 (25) | 12900 (16) | 24.4 (23) | NA |
| | 12 | F | 4470 (13) | 17100 (11) | NA | 1.40 (20) |
| | | M | 5650 (11) | 19000 (4) | NA | 1.50 (14) |

a: N = 6/sex/dose group, unless otherwise noted
b: R = AUC(0-7 days)_{Dosing interval 12} + AUC(0-7 days)_{Dosing interval 1}
c: NA = Not applicable
d: N = 5 [symbol]
indicates data missing or illegible when filed

TABLE 15
Mean (CV) LCF/HCA toxicokinetic parameters for animals
from the four month post dose period (days 84-207)
following intravenous bolus administration of 10, 30 or
100 mg/kg LCF/HCA to male and female cynomolgus
monkeys.

| [symbol] | | t [symbol] | CIₛₛ | Vₛₛ | MRT(I) |
|---|---|---|---|---|---|
| Dose (mg/kg) | Sex | (day) | (mL/day/kg) | (mL/kg) | (day) |
| 10 | F | 8.73 (41) | 6.60 (24) | 74.4 (12) | 11.5 (12) |
| | M | 10.1 (13) | 6.04 (5) | 74.1 (13) | 12.3 (13) |
| 30 | F | 10.4 (10) | 5.73 (8) | 64.4 (9) | 11.3 (16) |
| | M | 10.7 (44) | 5.22 (17) | 57.1 (13) | 11.3 (26) |
| 100 | F | 10.4 (29) | 6.03 (14) | 77.6 (31) | 12.7 (20) |
| | M | 12.7 (9) | 5.21 (2) | 73.2 (14) | 14.1 (15) |

a N = 3/dose group/sex [symbol]
indicates data missing or illegible when filed

TABLE 16
Mean (CV) serum concentrations for dosing interval 1 (days 0-7) and
dosing interval 12 (days 84-91) following intravenous bolus
administration of 10, 30 or 100 mg/kg LCF/HCA to cynomolgus
monkeys (males and females combined)

| [symbol] | Time [symbol] | | |
|---|---|---|---|
| Dose (mg/kg) | (Day) | Dosing Interval 1 | Dosing Interval 12 |
| 10 | 1 [symbol] | 303 (10) | 442 (50) |
| | 8 [symbol] | 212 (10) | 243 (48) |
| | 1 | 171 (12) | 189 (48) |
| | 2 | 137 (12) | 165 (50) |
| | 3 | 96.3 (15) | 242 (49) |
| | 4 | 90.0 (13) | 149 (49) |
| | 7 | 69.0 (14) | 173 (52) |
| 30 | 1 [symbol] | 893 (10) | 2120 (20) |
| | 8 [symbol] | 555 (11) | 1190 (12) |
| | 1 | 664 (32) | 779 (11) |
| | 2 | 453 (11) | 640 (12) |
| | 3 | 391 (13) | 880 (19) |
| | 4 | 385 (15) | 722 (21) |
| | 7 | 229 (17) | 663 (19) |
| 100 | 1 [symbol] | 4080 (23) | 4870 (15) |
| | 8 [symbol] | 3090 (34) | 3740 (10) |
| | 1 | 2400 [symbol] (16) | 2820 (7) |
| | 2 | 1900 (21) | 2520 (14) |
| | 3 | 1660 (11) | 3040 (12) |
| | 4 | 1140 (36) | 2220 (14) |
| | 7 | 1620 (24) | 1980 (33) |

a: N = 12/dose group, unless otherwise noted
b: time units are days, unless otherwise noted
c: time units are hours
d: LCF/HCA serum concentration units are mcg/ml
e: N = 11 [symbol]
indicates data missing or illegible when filed

TABLE 17
Mean (CV) serum concentrations for animals from the four month
postdose period (days 84-207) following intravenous bolus
administration of 10, 30 or 100 mcg/ml LCF/HCA to cynomolgus
monkeys (males and females combined).

| | [symbol] Dose (mg/kg) [symbol] | | | | | |
|---|---|---|---|---|---|---|
| Time (Day) [symbol] | 10 | | 30 | | 100 | |
| 1 [symbol] | 557 [symbol] | (18) | 2110 | (26) | 4950 | (17) |
| 8 [symbol] | 303 | (11) | 1190 | (12) | 3940 | (10) |
| 1 | 231 | (12) | 755 | (10) | 2850 | (9) |
| 2 | 194 | (15) | 630 | (12) | 2480 | (11) |
| 3 | 297 | (15) | 925 | (22) | 3060 | (12) |
| 4 | 187 | (18) | 724 | (23) | 2310 | (12) |
| 7 | 205 | (18) | 632 | (19) | 1760 | (47) |
| 10 | 118 | (17) | 349 | (22) | 1360 | (14) |
| 14 | 86.4 | (21) | 260 | (29) | 1120 | (16) |
| 26 | 25.9 | (38) | 99.7 | (47) | 343 | (37) |
| 42 | 9.97 | (83) | 34.0 | (64) | 177 | (49) |
| 56 | 1.26 | (60) | 10.6 [symbol] | (68) | 77.0 | (62) |
| 70 | 0.841 | (66) | 4.94 [symbol] | (52) | 32.5 | (99) |
| 84 | 0.434 | (81) | 3.11 [symbol] | (89) | 15.4 | (98) |
| 98 | 0.233 | (76) | 1.55 [symbol] | (78) | 9.99 [symbol] | (95) |
| 112 | 0.114 [symbol] | (93) | 1.22 [symbol] | (82) | 5.19 [symbol] | (102) |
| 123 | 0.0880 | (107) | 0.853 [symbol] | (80) | 2.82 | (53) |

a: N = 6/dose group, unless otherwise noted
b: time units are days (following the final dose on day 84), unless otherwise noted
c: time units are hours
d: LCF/HCA serum concentration units are mcg/ml
e: N = 5
f: N = 4 [symbol]
indicates data missing or illegible when filed

TABLE 18
Individual LCF/HCA toxicological parameters for dosing interval 1 (days 0-7)
and dosing interval 12 (days 84-91) following intravenous bolus administration of
10, 30 or 100 mg/kg LCF/HCA to cynomolgus monkeys.

| [symbol] | Dosing | | | C [symbol] | Cmax | Tmax | AUC (0-7 days) | νd ( [symbol] ) | |
|---|---|---|---|---|---|---|---|---|---|
| Dose (mg/kg) | Interval | Sex | Subject | (μg/mL) | (μg/mL) | (day) | (μg day/mL) | (mL/kg) | R [symbol] |
| 10 | 1 | F | 1501 | 263 | 271 | 0.0417* | 854 | 38.3 | NA [symbol] |
| | | | 1502 | 268 | 279 | 0.0417 | 812 | 34.7 | NA |
| | | | 1503 | 357 | 328 | 0.0417 | 783 | 25.0 | NA |
| | | | 1504 | 298 | 256 | 0.0417 | 770 | 33.6 | NA |
| | | | 1505 | 350 | 341 | 0.0417 | 927 | 27.8 | NA |
| | | | 1506 | 382 | 343 | 0.0417 | 934 | 27.8 | NA |
| | | M | 1001 | 275 | 251 | 0.0417 | 851 | 35.3 | NA |
| | | | 1002 | 301 | 257 | 0.0417 | 743 | 33.2 | NA |
| | | | 1003 | 364 | 348 | 0.0417 | 903 | 27.4 | NA |
| | | | 1004 | 337 | 320 | 0.0417 | 854 | 29.7 | NA |
| | | | 1005 | 325 | 308 | 0.0417 | 810 | 30.7 | NA |
| | | | 1006 | 313 | 297 | 0.0417 | 773 | 32.0 | NA |
| | 12 | F | 1501 | 531 | 494 | 0.0417 | 1690 | NA | 1.97 |
| | | | 1502 | 535 | 457 | 0.0417 | 1190 | NA | 1.47 |
| | | | 1503 | 533 | 604 | 0.0417 | 1270 | NA | 1.63 |
| | | | 1504 | 554 | 596 | 0.0417 | 1550 | NA | 2.18 |
| | | | 1505 | NA | NA | NA | NA | NA | NA |
| | | | 1506 | 891 | 633 | 0.0417 | 1860 | NA | 1.99 |
| | | M | 1001 | 418 | 3 [symbol] 6 | 0.0417 | 1650 | NA | 2.54 |
| | | | 1002 | 435 | 413 | 0.0417 | 1560 | NA | 2.10 |
| | | | 1003 | 487 | 452 | 0.0417 | 1460 | NA | 1.63 |
| | | | 1004 | 560 | 519 | 0.0417 | 1700 | NA | 1.97 |
| | | | 1005 | NA | NA | NA | NA | NA | NA |
| | | | 1006 | 780 | [symbol] | 0.0417 | 1710 | NA | 2.20 |

a: R = AUC (0-7 days) Dosing interval 12 [symbol] AUC (0-7 days) Dosing [symbol]
b: 0.0417 days = 1 hr
c: NA = Not applicable

| [symbol] | Dosing | | | C [symbol] | Cmax | Tmax | AUC (0-7 days) | νd ( [symbol] ) | |
|---|---|---|---|---|---|---|---|---|---|
| Dose (mg/kg) | Interval | Sex | Subject | (μg/mL) | (μg/mL) | (day) | (μg day/mL) | (mL/kg) | R [symbol] |
| 30 | 1 | F | 2501 | 928 | 999 | 1.0000 | 3280 | 32.3 | NA [symbol] |
| | | | 2502 | 599 | 921 | 1.0000 | 3350 | 33.4 | NA |
| | | | 2503 | 916 | 1080 | 0.0417 | 3830 | 32.6 | NA |
| | | | 2504 | 899 | 833 | 0.0417 | 2620 | 33.4 | NA |
| | | | 2505 | 1050 | 959 | 0.0417 | 3360 | 25.5 | NA |
| | | | 2506 | 1010 | 952 | 0.0417 | 2750 | 29.8 | NA |
| | | M | 901 | 1210 | 1090 | 0.0417 | 3300 | 24.7 | NA |
| | | | 902 | 936 | 873 | 0.0417 | 2550 | 32.0 | NA |
| | | | 903 | 923 | 991 | 1.0000 | 2670 | 33.2 | NA |
| | | | 904 | 937 | 1220 | 1.0000 | 3490 | 34.8 | NA |
| | | | 2005 | 1010 | 1330 | 1.0000 | 3310 | 29.8 | NA |
| | | | 2006 | 548 | 800 | 0.0417 | 2540 | 35.4 | NA |
| | 12 | F | 2501 | 2110 | 1930 | 0.0417 | 5210 | NA | 1.54 |
| | | | 2502 | 2430 | 2190 | 0.0417 | 5470 | NA | 1.63 |
| | | | 2503 | 3000 | 2560 | 0.0417 | 5540 | NA | 1.60 |
| | | | 2504 | 3630 | 3090 | 0.0417 | 4790 | NA | 1.63 |
| | | | 2505 | 2270 | 2120 | 0.0417 | 5560 | NA | 1.66 |
| | | | 2506 | 1730 | 1650 | 0.0417 | 5500 | NA | 2.00 |
| | | M | 901 | 2300 | 2180 | 0.0417 | 6290 | NA | 1.91 |
| | | | 902 | 1840 | 1570 | 0.0417 | 6210 | NA | 2.15 |
| | | | 903 | 2270 | 2040 | 0.0417 | 4400 | NA | 1.83 |
| | | | 904 | 2020 | 1900 | 0.0417 | 6510 | NA | 1.86 |
| | | | 2005 | 1990 | 1850 | 0.0417 | 6550 | NA | 1.98 |
| | | | 2006 | 2370 | 2230 | 0.0417 | 4820 | NA | 1.83 |

a: R = AUC (0-7 days) Dosing interval 12 [symbol] AUC (0-7 days) Dosing [symbol]
b: NA = Not applicable

| [symbol] | Dosing | | | C [symbol] | Cmax | Tmax | AUC (0-7 days) | νd ( [symbol] ) | |
|---|---|---|---|---|---|---|---|---|---|
| Dose (mg/kg) | Interval | Sex | Subject | (μg/mL) | (μg/mL) | (day) | (μg day/mL) | (mL/kg) | R [symbol] |
| 100 | 1 | F | 3501 | 4770 | 5490 | 0.3333 [symbol] | 15400 | 21.0 | NA [symbol] |
| | | | 3502 | 6180 | 5540 | 0.0417 | 12500 | 15.2 | NA |
| | | | 3503 | 3680 | 3630 | 0.0417 | 12000 | 27.2 | NA |
| | | | 3504 | 4130 | 3650 | 0.0417 | 10800 | 24.2 | NA |
| | | | 3505 | 3480 | 3410 | 0.0417 | 11800 | 25.7 | NA |
| | | | 3506 | 3220 | 3200 | 0.0417 | 11800 | 30.5 | NA |
| | | M | 3001 | 3620 | 3440 | 0.0417 | 12200 | 27.5 | NA |
| | | | 3002 | 5420 | 5320 | 0.0417 | 13800 | 15.4 | NA |
| | | | 3003 | 3270 | 3050 | 0.0417 | 10400 | 30.5 | NA |
| | | | 3004 | 4010 | 3710 | 0.0417 | 12300 | 25.0 | NA |
| | | | 3005 | 5890 | 5470 | 0.0417 | 12500 | 17.0 | NA |
| | | | 3006 | 3580 | 3520 | 0.0417 | 16400 | 25.1 | NA |
| | 12 | F | 3501 | 4010 | 3910 | 0.0417 | 15500 | NA | 1.01 |
| | | | 3502 | 3980 | 3870 | 0.0417 | 16500 | NA | 1.30 |
| | | | 3503 | 5570 | 5310 | 0.0417 | 19000 | NA | 1.59 |
| | | | 3504 | 4520 | 4450 | 0.0417 | 19400 | NA | 1.80 |
| | | | 3505 | 4330 | 4260 | 0.0417 | 17500 | NA | 1.45 |
| | | | 3506 | 4400 | 4370 | 0.0417 | 14500 | NA | 1.24 |
| | | M | 3001 | 4790 | 4830 | 0.0417 | 15100 | NA | 1.49 |
| | | | 3002 | 5000 | 5770 | 0.0417 | 18900 | NA | 1.39 |
| | | | 3003 | 5170 | 4870 | 0.0417 | 18400 | NA | 1.75 |
| | | | 3004 | 6340 | 5000 | 0.0417 | 19500 | NA | 1.69 |
| | | | 3005 | 6120 | 5710 | 0.0417 | 19900 | NA | 1.60 |
| | | | 3006 | 5620 | 5270 | 0.0417 | 19200 | NA | 1.17 |

a: R = AUC (0-7 days) Dosing interval 12 [symbol] AUC (0-7 days) Dosing [symbol]
b: 0.3333 days = 8 hr
c: NA = Not applicable [symbol]
indicates data missing or illegible when filed

TABLE 19
Individual LCF/HCA toxicokinetic parameters for animals from the four
month postdose period (days 84-207) following intravenous bolus administration of
10, 30 or 100 mg/kg LCF/HCA to cynomolgus monkeys.

| | | | Lambda 2 | | | | |
|---|---|---|---|---|---|---|---|
| | | | lower-upper | t [symbol] | CI [symbol] | V [symbol] | |
| Dose (mg/kg) | Sex | Subject | (day) [symbol] | (day) | (mL/day/kg) | (mL/kg) | MRT(l) (day) |
| 10 | F | 1502 | 7-56 | 4.57 | 8.41 | 84.5 | 10.0 |
| | | 1504 | 7-123 | 10.1 | 6.02 | 59.9 | 11.6 |
| | | 1506 | 10-123 | 11.4 | 5.37 | 58.9 | 12.8 |
| | M | 1002 | 10-123 | 10.1 | 6.39 | 77.2 | 12.1 |
| | | 1004 | 10-123 | 11.5 | 5.87 | 52.1 | 14.0 |
| | | 1006 | 7-112 | 5.50 | 5.85 | 83.0 | 10.8 |
| 30 | F | 2502 | 10-123 | 11.5 | 5.48 | 59.0 | 12.8 |
| | | 2504 | 10-123 | 9.62 | 6.26 | 58.0 | 9.25 |
| | | 2508 | 7-112 | 10.1 | 5.45 | 66.2 | 12.1 |
| | M | 902 | 10-123 | 13.7 | 4.83 | 64.5 | 13.3 |
| | | 904 | 10-123 | 13.0 | 4.61 | 57.8 | 12.5 |
| | | 2006 | 3-42 | 5.25 | 6.22 | 49.1 | 7.69 |
| 100 | F | 3502 | 3-112 | 7.07 | 6.07 | 53.4 | 10.4 |
| | | 3504 | 3-123 | 11.5 | 5.16 | 53.8 | 12.4 |
| | | 3506 | 10-123 | 12.8 | 6.85 | 106 | 15.4 |
| | M | 3002 | 7-123 | 11.8 | 5.30 | 73.3 | 13.5 |
| | | 3004 | 7-123 | 14.0 | 5.10 | 83.6 | 16.4 |
| | | 3006 | 10-123 | 12.5 | 6.22 | 52.9 | 12.0 |

[symbol]

: Days following the last dose (on Day 84) used to calculate lambda 2 [symbol]
indicates data missing or illegible when filed

### Example 6

### Pharmacokinetic Study of LCF/HCA in Healthy Human Subjects

This is a randomized, third-party blind (within dose group), rising, parallel group study to determine the safety, tolerability, and single-dose pharmacokinetics of anti-IGF1R antibody LCF/HCA (IgG1) in healthy human volunteers. The pharmaceutical composition used is described and discussed above e.g., in Example 1 under “Materials” (pH 5.5). Five dose levels of anti-IGF1R antibody LCF/HCA (IgG1) (0.3 mg/kg, 1.0 mg/kg, 3 mg/kg, 10 mg/kg and 20 mg/kg) were administered as a single dose by 1 hour intravenous infusion. Volumes of each dose given were as follows: 0.3 mg/kg: 80 ml; 1.0 mg/kg: 160 ml; 3 mg/kg: 80 ml; 10 mg/kg: 128 ml; and 20 mg/kg: 240 ml. Within each cohort, 6 subjects were randomized to receive anti-IGF1R antibody LCF/HCA (IgG1) and two subjects were randomized to receive placebo. Serial sampling was conducted to evaluate anti-IGF1R antibody LCF/HCA (IgG1) concentrations in serum. Briefly, the mean concentrations for doses of 10 mg/kg and 20 mg/kg remain above the target concentration of 19 ug/mL for a minimum of 4 weeks whereas the mean concentrations for doses lower than 10 mg/kg fall below the target trough concentration with 9 days of dose administration. As is discussed above, 19 μg/mL blood concentration of the antibody proved to be effective at inhibition of tumor cell growth in xenograft models. Therefore, the pharmacokinetic results support a dose of 10 mg/kg or higher to maintain a trough concentration of 19 ug/mL or greater upon repeat dosing when administered every 2 or 3 weeks.

The present invention includes methods for treating any medical disorder mediated by IGF1R expression or activity or IGF-1 or IGF-2 expression or activity by administering an anti-IGF1R antibody (e.g., LCF/HCA, for example a pharmaceutical composition thereof) to a subject in need of such a treatment wherein the anti-IGF1R treatment regimen achieves a pharmacokinetic profile associated with any of the doses set forth in Table 20 or 21, in particular a dose of about 10 or 20 mg/kg of body weight. For example, the methods of the invention include those wherein the pharmacokinetic profile achieved comprises any one, all or any combination of the elements set forth in Table 20 or 21 (e.g., Cmax, Tmax, AUC, t½ and serum:interstitial fluid ratio or any 1, 2, 3 or 4 of these factors in any combination whatsoever at about or at exactly the quantity shown in the table). Pharmaceutical compositions, such as unit dosage forms, which may, when administered to a subject with such a medical condition, achieve such a pharmacokinetic profile are also part of the present invention.

TABLE 20
Mean (% CV) Pharmacokinetic Parameters Following a
Single IV Infusion of 0.3, 1, 3, 10, or 20 mg/kg anti-IGF1R
antibody LCF/HCA to Healthy Volunteers

| | | | | | AUC (tf)^{b} | | | | |
|---|---|---|---|---|---|---|---|---|---|
| Dose | Cmax | | Tmax^{a} | | (μg · day/ | | t½,_{eff} | | Serum:interstitial |
| (mg/kg) | (μg/mL) | | (hr) | | mL) | | (day) | | fluid ratio |
| 0.3 | 7.54 | (25) | 2 | (1-2) | 17.2 | (30)^{c} | 1.48 | (10) | 5.29 |
| 1 | 29.4 | (26) | 1 | (1-2) | 130 | (25)^{d} | 3.70 | (9) | 3.16 |
| 3 | 94.7 | (18) | 1.5 | (1-2) | 646 | (13) | 7.29 | (10) | 3.35 |
| 10 | 263 | (18) | 1.5 | (1-6) | 2620 | (17) | 9.85 | (3) | 3.26 |
| 20^{e} | 526 | (11) | 2 | (1-2) | 5360 | (7) | 9.29 | (12) | 3.17 |
| 10^{f} | 354 | (15) | 2 | (1-12) | 2930 | (18) | 9.45 | (9) | NA |

n = 6 per dose group;
NA = not applicable
^{a}median (range)
^{b}tf = 56 days unless otherwise specified
^{c}median tf = 7 days
^{d}median tf = 28 days
^{e}n = 5
^{f}multiple doses of rHu-IGF-1 were co-administered to these subjects

TABLE 21
Mean Serum anti-IGF1R antibody LCF/HCA Concentration-Time Profiles
Following a Single IV Infusion of 0.3, 1, 3, 10, or 20 mg/kg anti-IGF1R
antibody LCF/HCA to Healthy Volunteers
anti-IGF1R antibody LCF/HCA (ug/mL)

| | | | | | | | | | | | 10 mg/kg + | |
|---|---|---|---|---|---|---|---|---|---|---|---|---|
| | 0.3 mg/kg | | 1 mg/kg | | 3 mg/kg | | 10 mg/kg | | 20 mg/kg^{a} | | rHu-IGF-1 | |
| Day | Mean | CV | Mean | CV | Mean | CV | Mean | CV | Mean | CV | Mean | CV |
| 0 | 0 | NA | 0 | NA | 0 | NA | 0 | NA | 0 | NA | 0 | NA |
| 0.0417 | 7.08 | 20 | 29.1 | 28 | 93.5 | 19 | 257 | 19 | 519^{b} | 9 | 323 | 10 |
| 0.0833 | 7.44 | 25 | 28.6 | 26 | 93.1 | 17 | 243 | 17 | 514 | 11 | 343 | 17 |
| 0.25 | 6.32 | 25 | 25.1 | 20 | 83.3 | 19 | 227 | 19 | 455^{b} | 8 | 303 | 11 |
| 0.5 | 5.40 | 28 | 22.0 | 30 | 70.6 | 15 | 189 | 20 | 423^{b} | 9 | 273 | 13 |
| 1 | 4.47 | 23 | 19.3 | 19 | 62.0 | 13 | 171 | 20 | 336^{b} | 9 | 213 | 20 |
| 2 | 3.09 | 27 | 14.3 | 29 | 48.9 | 10 | 134 | 17 | 311^{b} | 15 | 154 | 21 |
| 3 | 2.15 | 27 | 11.3 | 22 | 39.1 | 12 | 116 | 17 | 239^{b} | 3 | 132 | 10 |
| 5 | 1.21 | 34 | 8.40 | 27 | 31.2 | 10 | 91.9 | 26 | 239^{b} | 24 | 109 | 16 |
| 7 | 0.42 | 70 | 7.73 | 19 | 29.0 | 15 | 94.4 | 19 | 199 | 7 | 112 | 24 |
| 9 | 0.04 | 207 | 5.56 | 25 | 22.7 | 12 | 84.4 | 21 | 203 | 17 | 95.5 | 23 |
| 14 | 0 | NA | 2.19 | 30 | 15.1 | 14 | 56.6 | 14 | 127 | 14 | 61.2 | 20 |
| 17 | 0 | NA | 1.29 | 49 | 12.9 | 12 | 65.5 | 24 | 93.4 | 15 | 74.8 | 32 |
| 21 | 0 | NA | 0.26 | 96 | 9.17 | 17 | 51.1 | 19 | 83.8 | 13 | 58.5 | 37 |
| 28 | 0 | NA | 0.03 | 84 | 4.15 | 25 | 34.1 | 15 | 56.5 | 20 | 33.4 | 29 |
| 31 | NC | NA | NC | NA | NC | NA | NC | NA | NC | NA | 29.1 | 30 |
| 56 | 0 | NA | 0.01 | 245 | 0.47 | 131 | 2.24 | 30 | 11.3 | 39 | 2.39 | 63 |

n = 6 subjects per dose group unless otherwise specified
NA: not applicable (mean = 0 or NC);
NC: not collected
^{a}n = 5
^{b}n = 4; bioanalytical values were not reportable

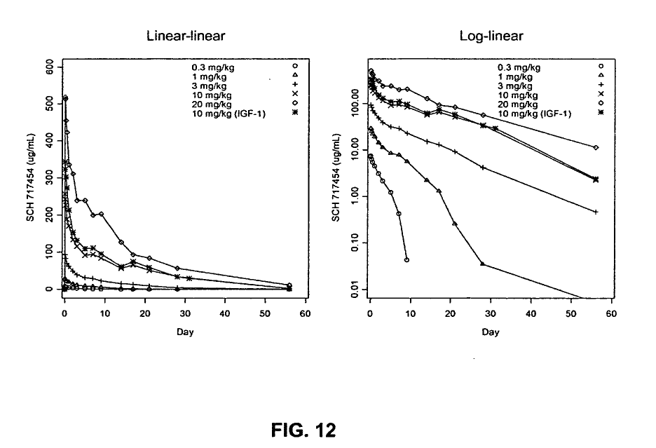

A graphical representation of blood concentrations of the antibody, at each dose tested, over time is set forth in FIG. 12.

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

We claim:

1. A method for treating or preventing a medical condition mediated by expression or activity of IGF1R comprising administering a dosage of an antibody or antigen-binding fragment thereof which binds specifically to IGF1R which dosage amount and frequency achieves and maintains a blood concentration of at least about 19 μg/mL.

2. The method of claim 1 wherein the dosage is about 10 mg/kg body weight or more; administered once every 3 weeks or more frequently.

3. The method of claim 1 wherein the medical condition is a member selected from the group consisting of osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

4. The method of claim 1 wherein said antibody or fragment comprises one or more members selected from the group consisting of:
(a) CDR-L1, CDR-L2 and CDR-L3 of the variable region of the 19D12/15H12 light chain immunoglobulin, and
(b) CDR-H1, CDR-H2 and CDR-H3 of the variable region of the 19D12/15H12 heavy chain immunoglobulin.

5. The method of claim 1 wherein the antibody or antigen-binding fragment thereof comprises a light chain immunoglobulin comprising complementarity determining regions comprising the amino acid sequences:
RASQSIGSSLH (SEQ ID NO: 1);
YASQSLS (SEQ ID NO: 2); and
HQSSRLPHT (SEQ ID NO: 3);
and a heavy chain immunoglobulin comprising complementarity determining regions comprising the amino acid sequences:
SFAMH (SEQ ID NO: 4);
VIDTRGATYYADSVKG (SEQ ID NO: 6); and
LGNFYYGMDV (SEQ ID NO: 7).

6. The method of claim 1 wherein the antibody or antigen-binding fragment thereof comprises:
(a) a light chain immunoglobulin comprising a mature fragment of the amino acid sequence set forth in SEQ ID NO: 8, 9, 10, 11, 12, 13 or 14; or
(b) a heavy chain immunoglobulin comprising a mature fragment of the amino acid sequence set forth in SEQ ID NO: 15, 16 or 17;
or both.

7. The method of claim 1 wherein the antibody or antigen-binding fragment thereof comprises a light chain immunoglobulin comprising amino acids 20-128 of the amino acid sequence set forth in SEQ ID NO: 14 and a heavy chain immunoglobulin comprising amino acids 20-137 of the amino acid sequence set forth in SEQ ID NO: 16.

8. The method of claim 7 wherein the antibody or fragment is a monoclonal antibody.

9. The method of claim 1 wherein the antibody or antigen-binding fragment thereof is administered in association with a further chemotherapeutic agent.

10. The method of claim 9 wherein the further chemotherapeutic agent is one or more members selected from the group consisting of: 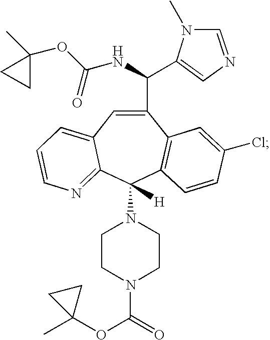 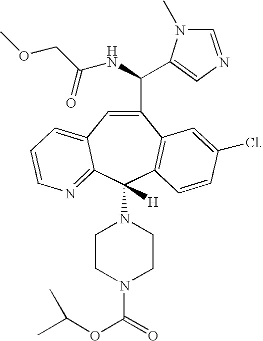
BMS-214662 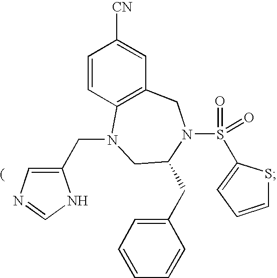
tipifarnib; HuMax-CD20; HuMax-EGFr; bevacizumab; Ibritumomab tiuxetan; a mixture of tositumomab and Iodine I¹³¹; gemtuzumab ozogamicin; MDX-010; CP-724714; TAK-165; HKI-272; gefitinib; erlotinib; calcitriol, lapatanib; GW2016; canertinib; ABX-EGF antibody; cetuximab; EKB-569; PKI-166; GW-572016; PD166285; goserelin acetate; triptorelin pamoate; the FOLFOX regimen; 5′-deoxy-5-fluorouridine; Asparaginase; Bacillus Calmette-Guerin (BCG) vaccine; bleomycin; buserelin; busulfan; oxaliplatin; JM118; JM383; JM559; JM518; 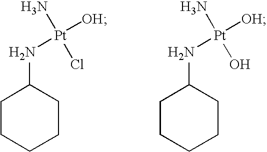
satraplatin; carboplatin; diethylstilbestrol; estradiol; conjugated estrogens; cladribine; clodronate; cyclophosphamide; cyproterone; cytarabine; dacarbazine; dactinomycin; PTK787; ZK 222584; VX-745; PD 184352; rapamycin; or temsirolimus; LY294002; LY292223; LY292696; LY293684; LY293646; sorafenib; ZM336372; L-779,450; flavopiridol; UCN-01; 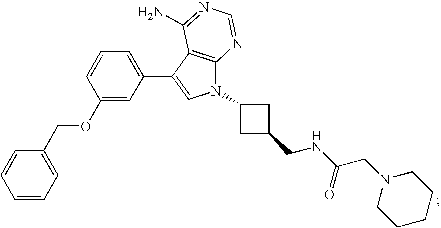
amifostine; NVP-LAQ824; suberoyl analide hydroxamic acid; valproic acid; trichostatin A; FK-228; SU11248; medroxyprogesterone acetate; hydroxyprogesterone caproate; 17-((1-Oxohexyl)oxy)pregn-4-ene-3,20-dione; carmustine; chlorambucil; octreotide; bortezomib; paclitaxel; docetaxel; vincristine; vinblastine; epothilone B; BMS-247550; etoposide; BMS-310705; temozolomide; 8-carbamoyl-3-methyl-[3H]-imidazo[5,1-d]-1,2,3,5-tetrazin-4-one; 8-carbamoyl-3-n-propyl-[3H]-imidazo[5,1-d]-1,2,3,5-tetrazin-4-one; 8-carbamoyl-3-(2-chloroethyl)-[3H]-imidazo-[5,1-d]-1,2,3,5-tetrazin-4-one; 3-(2-chloroethyl)-8-methylcarbamoyl-[3H]-imidazo[5,1-d]-1,2,3,5-tetrazin-4-one; 8-carbamoyl-3-(3-chloropropyl)-[3H]-imidazo-[5,1-d]-1,2,3,5-tetrazin-4-one; 8-carbamoyl-3-(2,3-dichloropropyl)-[3H]-imidazo[5,1-d]-1,2,3,5-tetrazin-4-one; 3-allyl-8-carbamoyl-[3H]-imidazo[5,1-d]-1,2,3,5-tetrazin-4-one; 3-(2-chloroethyl)-8-dimethylcarbamoyl-[3H]-imidazo[5,1-d]-1,2,3,5-tetrazin-4-one; 3-(2-bromoethyl)-8-carbamoyl-[3H]-imidazo-[5,1-d]-1,2,3,5-tetrazin-4-one; 3-benzyl-8-carbamoyl-[3H]-imidazo[5,1-d]-1,2,3,5-tetrazin-4-one; 8-carbamoyl-3-(2-methoxyethyl)-[3H]-imidazo[5,1-d]-1,2,3,5-tetrazin-4-one; 8-carbamoyl-3-cyclohexyl-[3H]-imidazo[5,1-d]-1,2,3,5-tetrazin-4-one; 8-carbamoyl-3-(methoxybenzyl)-[3H]imidazo[5,1-d]-1,2,3,5-tetrazin-4-one; doxorubicin; daunorubicin; epirubicin; bicalutamide; flutamide; nilutamide; megestrol acetate; hydroxyurea; Idarubicin; ifosfamide; imatinib; leucovorin; leuprolide; levamisole; lomustine; mechlorethamine; melphalanm; mercaptopurine; mesna; methotrexate; mitomycin; mitotane; mitoxantrone; fludarabine; fludrocortisone; fluoxymesterone; KRN951; aminoglutethimide; amsacrine; anagrelide; droloxifene, 4-hydroxytamoxifen; tamoxifen; pipendoxifene; arzoxifene; raloxifene; fulvestrant; acolbifene; toremifine; lasofoxifene; idoxifene; bazedoxifene; HMR-3339; ZK-186619; anastrazole; letrozole; exemestane; gemcitabine HCl; 13-cis-retinoic acid; pamidronate; pentostatin; Plicamycin; porfimer; procarbazine; raltitrexed; Rituximab streptozocin; teniposide; testosterone; thalidomide; thioguanine; thiotepa; tretinoin vindesine; interferon alfa-2a; interferon alfa-2b; interferon alfa-2c; interferon alfa n-1; interferon alfa n-3; consensus interferon; albumin-interferon-alpha; camptothecin; topotecan; etoposide; irinotecan; AEW-541;

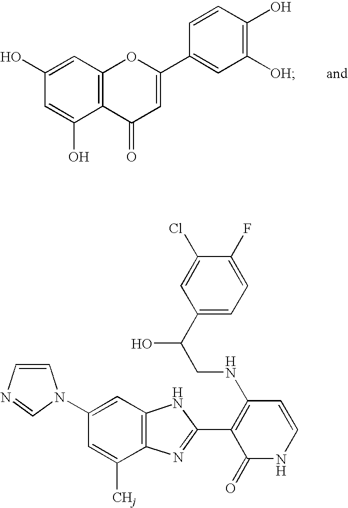

11. The method of claim 10 wherein the further chemotherapeutic agent is selected from the group consisting of:
lonafarnib;
cetuximab;
irinotecan;
erlotinib;
rapamycin;
temsirolimus;
sorafenib;
gefitinib;
fulvestrant;
octreotide;
temozolomide; and
4-hydroxytamoxifen.

12. The method of claim 1 wherein the antibody or antigen-binding fragment thereof is a monoclonal antibody.

13. The method of claim 1 wherein the antibody or antigen-binding fragment thereof is a labeled antibody, bivalent antibody, a polyclonal antibody, a bispecific antibody, a chimeric antibody, a recombinant antibody, an anti-idiotypic antibody, a humanized antibody or a bispecific antibody.

14. The method of claim 1 wherein the antibody or antigen-binding fragment thereof is a camelized single domain antibody, a diabody, an scfv, an scfv dimer, a dsfv, a (dsfv)₂, a dsFv-dsfv′, a bispecific ds diabody, an Fv, an Fab, an Fab′, an F(ab′)₂, or a domain antibody.

15. The method of claim 1 wherein the antibody or antigen-binding fragment thereof is linked to a constant region.

16. The method of claim 15 wherein the constant region is a κ light chain, γ1 heavy chain, γ2 heavy chain, γ3 heavy chain or γ4 heavy chain.

17. The method of claim 1 wherein the antibody or antigen-binding fragment thereof is an isolated antibody comprising a heavy chain encoded by a polynucleotide in plasmid 15H12/19D12 HCA (γ1) which is deposited at the American Type Culture Collection (ATCC) under number PTA-5216; and a light chain encoded by a polynucleotide in plasmid 15H12/19D12 LCF (κ) which is deposited at the American Type Culture Collection (ATCC) under number PTA-5220.

18. A unit dosage form comprising one or more doses of a pharmaceutically acceptable carrier and an antibody or antigen-binding fragment thereof comprising one or more members selected from the group consisting of:
(a) CDR-L1, CDR-L2 and CDR-L3 of the variable region of the 19D12/15H12 light chain immunoglobulin, and
(b) CDR-H1, CDR-H2 and CDR-H3 of the variable region of the 19D12/15H12 heavy chain immunoglobulin;
wherein said dose is sufficient to reach and maintain a 19 μg/mL blood concentration of said antibody or fragment when administered once every three weeks or more frequently.

19. The unit dosage form of claim 18 which is acceptable for parenteral administration.

20. The unit dosage form of claim 19 which is acceptable for administration by a route which is a member selected from the group consisting of intravenous, intramuscular, intratumoral, intrathecal, intraarterial and subcutaneous.

21. The unit dosage form of claim 19 which is aqueous.

22. The unit dosage form of claim 18 which is lyophilized.

23. A vial containing the unit dosage form of claim 18.

24. The vial of claim 23 which is a glass vial.

25. A hypodermic needle comprising the unit dosage form of claim 18.
